(19) United States
(12) Patent Application Publication
(10) Pub. No.: US 2010/0092484 A1
(43) Pub. Date: Apr. 15, 2010

(54) CD44 ANTIBODIES

(76) Inventors: Xu Xu, Winchester, MA (US); Vahe Bedian, Framingham, MA (US); Erika Meaddough, Gilroy, CA (US); Haichun Huang, Fremont, CA (US); Lan Yang, Morgan Hill, CA (US); Kristopher Toy, San Jose, CA (US); Mohan Srinivasan, San Mateo, CA (US); Advait V. Badkar, Wildwood, MO (US)

Correspondence Address: DICKSTEIN SHAPIRO LLP, 1825 EYE STREET NW, Washington, DC, 20006-5403, US

(21) Appl. No.: 12/518,856

(22) PCT Filed: Dec. 20, 2007
(86) PCT No.: PCT/US07/25975

§ 371 Date: Dec. 7, 2009

Related U.S. Application Data
(60) Provisional application No. 60/876,109, filed on Dec. 21, 2006.

(51) Int. Cl. A61K 39/395; C07K 16/18; A61P 29/00; A61P 37/00; C07H 21/04; C12N 15/63; C12N 5/10; C12N 5/12

(52) U.S. Cl. 424/158.1; 530/389.2; 536/23.53; 435/320.1; 435/325; 435/346; 435/358; 435/364

(57) ABSTRACT

The present invention relates to antibodies including human antibodies and antigen-binding portions thereof that bind to CD44, and that function to inhibit CD44. The invention also relates to heavy and light chain immunoglobulins derived from human CD44 antibodies and nucleic acid molecules encoding such immunoglobulins. The present invention also relates to methods of making human CD44 antibodies, compositions comprising these antibodies and methods of using the antibodies and compositions or medicaments for treatment.

### CROSS-REFERENCE TO RELATED PATENTS AND PATENT APPLICATIONS

This application claims the benefit of U.S. Provisional Patent Application Ser. No. 60/876,109 filed Dec. 21, 2006; which is incorporated by reference herein in its entirety.

### FIELD OF THE INVENTION

The present invention relates to antibodies and antigen-binding portions thereof that bind to human CD44. The invention also relates to nucleic acid molecules encoding such antibodies and antigen-binding portions, methods of making CD44 antibodies and antigen-binding portions, compositions comprising these antibodies or antigen-binding portions thereof and methods of using the antibodies, antigen-binding portions, and compositions or medicaments for treatment.

### BACKGROUND OF THE INVENTION

Inflammation, a local accumulation of fluid, caused by for example, physical injury, infection or an immune response, is initiated by the recruitment of inflammatory cells, such as monocytes and T-cells into the extracellular matrix. Naor, D. et al., (2003) Arthritis Res Ther, 5:105-115. This cellular recruitment typically results in the further infiltration and increase of cytokines, such as TNF-α, IL-6 and IL-1β, into the extracellular matrix (Ibid). Such recruitment and infiltration of cells, along with various other cellular processes, such as for example, regulation of growth, adhesion, differentiation, invasion and survival are mediated by transmembrane glycoprotein cell-adhesion molecules, a superfamily of adhesion receptors. Members of the cell adhesion receptor family include CD44, a broadly distributed class I transmembrane glycoprotein. CD44 plays a pivotal role in a variety of cellular behaviors, including adhesion, migration, activation, and survival. Ponta, H. et al., (2003) Molecular Cell Biology, 4:33-45.

CD44 ranges in molecular weight from 80 to 90 kDa and can generate close to 800 variant isoforms by differential alternative splicing. Cichy, J. et al., (2003) Journal of Cell Biology, 161:5, 839-843. At present several dozen isoforms are known. CD44 is ubiquitously expressed on many cell types including leukocytes, fibroblasts, epithelial cells, keratinocytes and some endothelial cells, with the standard CD44 (CD44s) form, which lacks any variant exons, being the most abundantly expressed isoform.

CD44 together with its primary ligand, hyaluronan or hyaluronic acid (HA), a hydrophilic, linear, extracellular polysaccharide, play a major role in inflammation. Naor D., (2003) Arthritis Res Ther, 5:105-115 and Aruffo, A. (1990) Cell 61, 1301-1313. For example, in an in vivo study a monoclonal anti-CD44 antibody, IRAWB14, which induces CD44-mediated HA-binding activity, resulted in the exacerbation of the inflammatory symptoms in mice with proteoglycan-induced arthritis. Pure, E. et al., (2001) TRENDS in Molecular Medicine, 7:213-221.

### SUMMARY OF THE INVENTION

The present invention provides isolated antibodies or antigen-binding portions thereof that specifically bind CD44 and may act as a CD44 antagonist, and compositions or medicaments comprising said antibody or antigen-binding portions thereof. Another aspect of the present invention provides any of the antibodies or antigen-binding portions thereof as described herein, where said antibody or antigen-binding portion is a human antibody. In a further aspect, said antibody or antigen-binding portion is a human recombinant antibody.

The invention provides antibodies that specifically bind CD44 comprising: (i) a heavy and/or light chain, or (ii) the variable domains thereof, or (iii) antigen-binding portions thereof, or (iv) complementarity determining region(s) (CDR) thereof.

The invention further provides CD44 antibodies or antigen-binding portions thereof wherein the antibody or antigen-binding portion thereof having at least one functional properties as described below in a) thru g).

a) binds to CD44 with a K_{D} of 1000 nM or less as measured by surface plasmon resonance;

b) has an off rate (k_{off}) for CD44 of less than or equal to 0.01^{s-1} as measured by surface plasmon resonance;

c) binds to CD44 with an EC₅₀ of less than 500 nM, 75 μg/ml as measured by FACS or ELISA binding assay;

d) inhibits the interaction between CD44 and HA with an IC₅₀ of less than 500 nM, 75 μg/ml as measured by an ELISA binding assay;

e) reduces the in vivo surface expression of CD44 receptors in inflammatory cells, such as CD3+T cells at an IC₅₀ of less than about 100 nM as measured by FACS;

f) reduces the surface expression of CD44 receptors in vitro with an IC₅₀ of less than 50 nM;

g) has a selectivity for CD44 over lymphatic vessel endothelial hyauronan receptor 1 protein (LYVE-1) by at least 100 fold.

In another embodiment, the invention provides an isolated nucleic acid molecule comprising a nucleotide sequence that encodes any of the antibodies or antigen binding portions thereof as described herein. In one particular embodiment, the invention provides an isolated nucleic acid molecule comprising a nucleotide sequence as set forth in any of the SEQ ID NOs described herein. The invention further provides a vector comprising any of the nucleic acid molecules described herein, wherein the vector optionally comprises an expression control sequence operably linked to the nucleic acid molecule.

Another embodiment provides a host cell comprising any of the vectors described herein or comprising any of the nucleic acid molecules described herein. The present invention also provides an isolated cell line that produces any of the antibodies or antigen-binding portions as described herein or that produces the heavy chain or light chain of any of said antibodies or said antigen-binding portions.

In another embodiment, the present invention provides a method for producing a CD44 antibody or antigen-binding portion thereof, comprising culturing any of the host cells or cell lines described herein under suitable conditions and recovering said antibody or antigen-binding portion.

The present invention also provides a non-human transgenic animal or transgenic plant comprising any of the nucleic acids described herein, wherein the non-human transgenic animal or transgenic plant expresses said nucleic acid.

The present invention further provides a method for isolating an antibody or antigen-binding portion thereof that binds to CD44, comprising the step of isolating the antibody from the non-human transgenic animal or transgenic plant as described herein.

The invention provides compositions comprising: (i) the heavy and/or light chain, the variable domains thereof, or antigen-binding portions thereof, or CRDs thereof, of said anti-CD44 antibody, or nucleic acid molecules encoding them; and (ii) a pharmaceutically acceptable carrier. Compositions of the invention may further comprise another component, such as a therapeutic agent or a diagnostic agent.

The present invention also provides a pharmaceutical composition or medicament comprising any of the antibodies or antigen-binding portions thereof as described herein and optionally a pharmaceutically acceptable carrier linked or in suspension. Compositions of the invention may further comprise another component, such as therapeutic agent or diagnostic agent.

Diagnostic and therapeutic methods are also provided by the invention.

The present invention also provides a method for treating inflammatory cell infiltration or recruitment in a mammal in need thereof, comprising the step of administering to said mammal any of the antibodies or antigen-binding portions thereof, or any of the pharmaceutical compositions, as described herein.

Another aspect of the present invention provides any of the antibodies or antigen-binding portions thereof as described herein, where said antibody or antigen-binding portion is a human antibody. In a further aspect, said antibody or antigen-binding portion is a human recombinant antibody.

### BRIEF DESCRIPTION OF THE DRAWINGS

FIG. 1 is a schematic representation of an Immunoglobulin (IgG).

FIG. 2 is a sequence alignment of predicted amino acid sequences of the heavy and light chain variable domains of isolated anti-CD44 monoclonal antibodies with the germline amino acid sequences of the corresponding light and heavy chain genes. Identical residues between the clones and the germline sequences are shown by dashes, deletions/insertions are shown by hash marks, mutations are listed, and CDRs are underlined.

FIG. 3 is a graph illustrating the anti-CD44 1A9.A6.B9 antibody blocking the binding of HA to the CD44-Ig fusion protein.

FIG. 4A-4C are graphs showing the binding of anti-CD44 antibodies to cells as assayed by flow cytometry sorting (FACS).

FIG. 4A is a graph illustrating binding of anti-CD44 1A9.A6.B9 and 14G9.B8.B4 antibodies to human whole blood T-cells as assayed by FACS.

FIG. 4B is a graph illustrating binding of anti-CD44 1A9.A6.B9 and 14G9.B8.B4 antibodies to cynomolgus monkey whole blood T-cells as assayed by FACS.

FIG. 4C is a graph illustrating binding of anti-CD44 antibodies to 300-19 cells transduced with human and cyano CD44 as assayed by FACS.

FIG. 5 is a graph illustrating the binding study of anti-CD44 1A9.A6.B9 antibody using human and cyno CD44-Ig fusion proteins as measured by ELISA assays.

FIG. 6 shows a graph illustrating anti-CD44 1A9.A6.B9 and 14G9.B8.B4 antibodies blocking the release of IL-1β stimulated by lipopolysaccharide (LPS) and HA from human whole monocytes, as quantitated using ELISA.

FIG. 7 is a graph showing anti-CD44 1A9.A6.B9 and 14G9.B8.B4 antibodies reducing the surface expression of CD44 receptors on CD3+ peripheral T cells as measured by FAGS.

FIG. 8A is a graph showing the reduction of surface expression of CD44 receptors on human peripheral leukocytes (lymphocytes) by anti-CD44 antibody 1A9.A6.B9.

FIG. 8B is a graph showing the reduction of surface expression of CD44 receptors on human peripheral leukocytes (monocytes) by anti-CD44 antibody 1A9.A6.B9.

FIG. 8C is a graph showing the reduction of surface expression of CD44 receptors on human peripheral neutrophils (PMNs) by anti-CD44 antibody 1A9.A6.B9.

FIGS. 9A and 9B show graphs illustrating the single dose in vivo study of anti-CD44 1A9.A6.B6 antibody administered to cynomolgus monkeys, as quantitated using FACS.

FIG. 10A is a graph illustrating the binding of anti-CD44 1A9.A6.B9 in direct competition with anti-CD44 antibody MEM 85 using human peripheral T-cells, as quantitated using FACS.

FIG. 10B is a graph illustrating the binding of anti-CD44 1A9.A6.B9 in direct competition with anti-CD44 antibody MEM 85 using the 300-19 cells transfected with human CD44 as described in EXAMPLE 1, as quantitated using FACS.

FIG. 11 is a graph illustrating the present aggregate formed (high molecular mass species (HMMS)) at 5° C. (11a), 25° C. (11b) and 40° C. (11c) as measured by SE-HPLC.

FIG. 12 is a graph showing the total acid species formed at 5° C. (12a), 25° C. (12b) and 40° C. (12c) as measured by iCE.

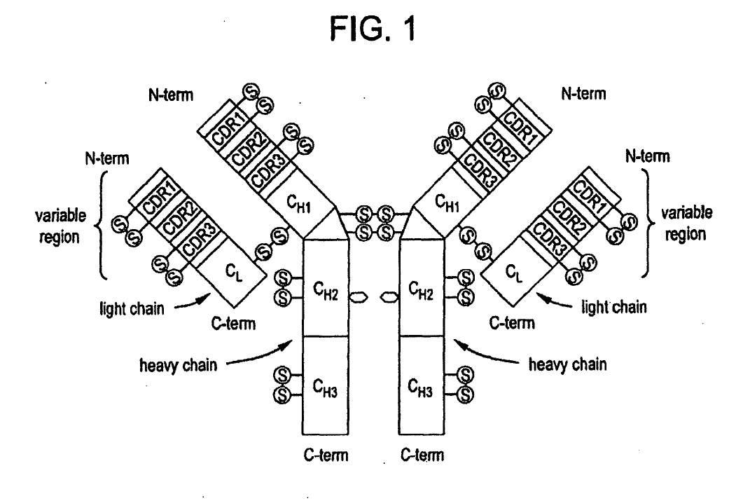

### DETAILED DESCRIPTION OF THE INVENTION

### Definitions

Throughout this specification and claims, the word “comprise,” or variations such as “comprises” or “comprising,” will be understood to imply the inclusion of a stated integer or group of integers but not the exclusion of any other integer or group of integers.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, the nomenclature used herein in connection with cell and tissue culture, molecular biology, immunology, microbiology, genetics and protein and nucleic acid chemistry and hybridization are those commonly used in the art.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 120 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. See generally, Fundamental Immunology Ch. 7 (Paul, W., ed., 2nd ed. Raven Press, N.Y. (1989)). The variable regions of each heavy/light chain pair (V_{H} and V_{L}), respectively, form the antibody binding site. Thus, an intact IgG antibody, for example, has two binding sites. Except in bifunctional or bispecific antibodies, the two binding sites are the same.

The variable regions of the heavy and light chains exhibit the same general structure of relatively conserved framework regions (FR) joined by three hyper variable regions, also called complementarity determining regions or CDRs. The term “variable” refers to the fact that certain portions of the variable domains differ extensively in sequence among antibodies and are used in the binding and specificity of each particular antibody for its particular antigen. The variability, however, is not evenly distributed throughout the variable domains of antibodies, but is concentrated in the CDRs, which are separated by the more highly conserved FRs. The CDRs from the two chains of each pair are aligned by the FRs, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883. As used herein, an antibody that is referred to by number is the same as a monoclonal antibody that is obtained from the hybridoma of the same number. For example, monoclonal antibody 1A9.A6.B9 is the same antibody as one obtained from hybridoma 1A9.A6.B9, or a subclone thereof. As used herein, a Fd fragment means an antibody fragment that consists of the V_{H} and C_{H}1 domains; a Fv fragment consists of the V_{L} and V_{H} domains of a single arm of an antibody; and a dAb fragment (Ward et al., (1989) Nature 341:544-546) consists of a V_{H} domain.

In some embodiments, the antibody is a single-chain antibody (scFv) in which V_{L} and V_{H} domains are paired to form a monovalent molecule via a synthetic linker that enables them to be made as a single protein chain. (Bird et al., (1988) Science 242:423-426 and Huston et al., (1988) Proc. Natl. Acad. Sci. USA 85:5879-5883). In some embodiments, the antibodies are diabodies, i.e., bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. (See e.g., Holliger P. et al., (1993) Proc. Natl. Acad. Sci. USA 90:6444-6448, and Poljak R. J. et al., (1994) Structure 2:1121-1123. In an embodiment, one or more CDRs from an antibody of the invention may be incorporated into a molecule either covalently or noncovalently to make it an immunoadhesin that specifically binds to CD44. In such embodiments, the CDR(s) may be incorporated as part of a larger polypeptide chain, may be covalently linked to another polypeptide chain, or may be incorporated noncovalently.

In antibody embodiments having one or more binding sites, the binding sites may be identical to one another or may be different.

The term “analog” or “polypeptide analog” as used herein refers to a polypeptide that comprises a segment that has substantial identity to some reference amino acid sequence and has substantially the same function or activity as the reference amino acid sequence. Typically, polypeptide analogs comprise a conservative amino acid substitution (or insertion or deletion) with respect to the reference sequence. Analogs can be at least 20 or 25 amino acids long, or can be at least 50, 60, 70, 80, 90, 100, 150 or 200 amino acids long or longer, and can often be as long as the full-length polypeptide. Some embodiments of the invention include polypeptide fragments or polypeptide analog antibodies with 1, 2, 3, 4, 5, 6, 7, 8, 9, 10, 11, 12, 13, 14, 15, 16 or 17 substitutions from the germline amino acid sequence. Fragments or analogs of antibodies or immunoglobulin molecules can be readily prepared by those of ordinary skill in the art following the teachings of this specification:

In an embodiment, amino acid substitutions to a CD44 antibody or antigen-binding portion thereof are those which: (1) reduce susceptibility to proteolysis, (2) reduce susceptibility to oxidation, (3) alter binding affinity for forming protein complexes, or (4) confer or modify other physicochemical or functional properties of such analogs, but still retain specific binding to CD44. Analogs can include various substitutions to the normally-occurring peptide sequence. For example, single or multiple amino acid substitutions, preferably conservative amino acid substitutions, may be made in the normally-occurring sequence, for example in the portion of the polypeptide outside the domain(s) forming intermolecular contacts. Amino acid substitutions can also be made in the domain(s) that form intermolecular contacts that can improve the activity of the polypeptide. A conservative amino acid substitution should not substantially change the structural characteristics of the parent sequence; e.g., a replacement amino acid should not alter the anti-parallel β-sheet that makes up the immunoglobulin binding domain that occurs in the parent sequence, or disrupt other types of secondary structure that characterizes the parent sequence. In general, glycine and proline would not be used in an anti-parallel β-sheet. Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et al., (1991) Nature 354:105.

As used herein, the term “antibody” is synonymous with immunoglobulin and is to be understood as commonly known in the art. In particular, the term antibody is not limited by any particular method of producing the antibody. For example, the term antibody includes, inter alia, recombinant antibodies, monoclonal antibodies, and polyclonal antibodies.

The term “antigen-binding portion” of an antibody (or simply “antibody portion” or “portion”), as used herein, refers to one or more fragments of an antibody that retain the ability to specifically bind to an antigen (e.g., CD44). It has been shown that the antigen-binding function of an antibody can be performed by fragments of a full-length antibody. Examples of binding fragments encompassed within the term “antigen-binding portion” of an antibody include: (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)); see e.g., Bird et al., (1988) Science 242:423-426 and Huston et al., (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger et al., (1993) Proc. Natl. Acad. Sci. USA 90:6444-6448; Poljak et al., (1994) Structure 2:1121-1123).

Still further, an antibody or antigen-binding portion thereof may be part of larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov et al., (1995) Human Antibodies and Hybridomas 6:93-101) and use of a cysteine residue, a marker peptide and a C-terminal polyhistidine tag to make bivalent and biotinylated scFv molecules (Kipriyanov et al., (1994) Mol. Immunol. 31:1047-1058). Other examples include where one or more CDRs from an antibody are incorporated into a molecule either covalently or noncovalently to make it an immunoadhesin that specifically binds to an antigen of interest, such as CD44. In such embodiments, the CDR(s) may be incorporated as part of a larger polypeptide chain, may be covalently linked to another polypeptide chain, or may be incorporated noncovalently. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

Unless specifically indicated otherwise, the term “CD44” refers to human CD44. CD44 is a multistructual extracellular matrix receptor and a member of the classical family of transmembrane glycoproteins that regulates cell-cell and cell-matrix activities. The cloning and sequence of a human CD44 has been reported, e.g. Arrofo, A. (1990) Cell, 16. (Accession No. NM_{—}001001391), and is set forth in SEQ ID NO:1. The term CD44 is intended to include recombinant human CD44 and recombinant chimeric forms of CD44, which can be prepared by standard recombinant expression methods or purchased commercially (e.g., R&D Systems Cat. No. 861-PC-100). Particularly, CD44 is an 80-90 kDa glycolylated type I transmembrane protein encoded by a single 60 kb gene comprising 20 exons. Ten of the 20 exons, (standard exons 1s to 10s) are expressed in all CD44 positive cells and encode the “standard CD44” or “CD44s”. The 10 other exons (variant exons 1v to 10v) are subjected to alternative splicing and encode peptidic sequences inserted in the extracellular domain of CD44s. The “extracellular domain of CD44” comprises an N-terminus globular region stabilized by 3 disulfide bonds, and is separated from the cellular membrane by a linear structure and is approximately 247 residues in length and is set forth in SEQ ID NO:3. (Gadhoum Z. et al., (2004) Leukemia & Lymphoma 45(8):1501-1510). This tri-disulfide bond ladder includes the globular region which displays the hyaluronic acid (HA, hyaluronate, hyaluronan) binding domain “HA binding domain”, located within the extracellular domain of CD44s and includes the “link module” which is approximately 100 residues in length, (residues 32-123 of the extracellular domain of CD44s and as set forth in SEQ ID NO:5). The “HA binding domain” may further be characterized as comprising at least amino acid residues Lys38, Arg41, Tyr42, Arg78, Tyr79, Asn100, Asn101, Arg150, Arg154 and Arg162. (Teriete P. et al., (2004) Molecular Cell, 13, 483-496).

The term “chimeric antibody” as used herein means an antibody that comprises regions from two or more different antibodies, including antibodies from different species. For example, one or more of the CDRs of a chimeric antibody can be derived from a human CD44 antibody. In one example, the CDRs from a human antibody can be combined with CDRs from a non-human antibody, such as mouse or rat. In another example, all of the CDRs can be derived from human CD44 antibodies. In another example, the CDRs from more than one human CD44 antibody can be combined in a chimeric antibody. For instance, a chimeric antibody may comprise a CDR1 from the light chain of a first human CD44 antibody, a CDR2 from the light chain of a second human CD44 antibody and a CDR3 from the light chain of a third human CD44 antibody, and CDRs from the heavy chain may be derived from one or more other CD44 antibodies. Further, the framework regions may be derived from one of the CD44 antibodies from which one or more of the CDRs are taken or from one or more different human antibodies. Further, the term “chimeric antibody” is intended to encompass any of the above mentioned combinations where the combinations involved human and non-human antibodies.

The term “compete”, as used herein with regard to an antibody, means that a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As the term is used herein, a “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence similarity may be adjusted upwards to correct for the conservative nature of the substitution. Means for making this adjustment are well-known to those of skill in the art. Pearson, (1994) Methods Mol. Biol. 243:307-31. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups can be, for example, valine-leucine-isoleucine, phenylalanine-tyrosine, lysine-arginine, alanine-valine, glutamate-aspartate, and asparagine-glutamine.

A conservative replacement is also any change having a positive value in the PAM250 log-likelihood matrix disclosed in Gonnet et al., (1992) Science 256:1443-45. A “moderately conservative” replacement is any change having a non-negative value in the PAM250 log-likelihood matrix.

“Contacting” refers to bringing an antibody or antigen binding portion thereof of the present invention and a target CD44, or epitope thereof, together in such a manner that the antibody can affect the biological activity of the CD44. Such “contacting” can be accomplished “in vitro,” e.g., in a test tube, a petri dish, or the like. In a test tube, contacting may involve only an antibody or antigen binding portion thereof and CD44 or epitope thereof or it may involve whole cells. Cells may also be maintained or grown in cell culture dishes and contacted with antibodies or antigen binding portions thereof in that environment. In this context, the ability of a particular antibody or antigen binding portion thereof to affect a CD44-related disorder, i.e., the IC₅₀ of the antibody, can be determined before use of the antibody in vivo with more complex living organisms is possible. For cells outside the organism, multiple methods exist, and are well-known to those skilled in the art, to contact CD44 with the antibodies or antigen-binding portions thereof.

As used herein, the term “ELISA” refers to an enzyme-linked immunosorbent assay. This assay is well known to those of skill in the art. Examples of this assay can be found in Vaughan, T. J. et al., (1996) Nat. Biotech. 14:309-314, as well as in EXAMPLES 5, 6, 7 and 11 of the present application.

The term “epitope” includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three dimensional structural characteristics, as well as specific charge characteristics. An epitope may be “linear” or “conformational.” In a linear epitope, all of the points of interaction between the protein and the interacting molecule (such as an antibody) occur linearally along the primary amino acid sequence of the protein. In a conformational epitope, the points of interaction occur across amino acid residues on the protein that are separated from one another. While, once a desired epitope on an antigen is determined, antibodies to that epitope can be generated, e.g., using the techniques described in the present invention. During the discovery process, the generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

As used herein, the term “germline” refers to the nucleotide sequences of the antibody genes and gene segments as they are passed from parents to offspring via the germ cells. This germline sequence is distinguished from the nucleotide sequences encoding antibodies in mature B cells which have been altered by recombination and hypermutation events during the course of B cell maturation. The germline antibodies of the present invention are designated as g-1A9.A6.B9, g-2D1.A3.D12 and g-14G9.B8.B4.

As used herein, the term “human antibody” means any antibody in which the variable and constant domain sequences are human sequences. The term encompasses antibodies with sequences derived from human genes, including those which have been changed, e.g., to decrease possible immunogenicity, increase affinity, eliminate cysteine residues that might cause undesirable folding, etc. The term also encompasses such antibodies produced recombinantly in non-human cells, which might impart glycosylation not typical of human cells. These antibodies may be prepared in a variety of ways, as described below.

As used herein, the term “humanized antibody” refers to antibodies of non-human origin, wherein the amino acid residues that are characteristic of antibody sequences of the non-human species are replaced with residues found in the corresponding positions of human antibodies. This “humanization” process is thought to reduce the immunogenicity in humans of the resulting antibody. It will be appreciated that antibodies of nonhuman origin can be humanized using techniques well known in the art. Winter et al., (1993) Immunol. Today 14:43-46. The antibody of interest may be engineered by recombinant DNA techniques to substitute the CH1, CH2, CH3, hinge domains, and/or the framework domain with the corresponding human sequence. PCT Publication No. WO 92/02190, and U.S. Pat. Nos. 5,530,101, 5,585,089, 5,693,761, 5,693,792, 5,714,350, and 5,777,085. The term “humanized antibody”, as used herein, further includes within its meaning, chimeric human antibodies and CDR-grafted antibodies. Chimeric human antibodies of the invention include the V_{H} and V_{L} of an antibody of a non-human species and the C_{H} and C_{L} domains of a human antibody. The CDR-transplanted antibodies of the invention result from the replacement of CDRs of the V_{H} and V_{L} of a human antibody with those of the V_{H} and V_{L}, respectively, of an antibody of an animal other than a human.

The term “isolated polynucleotide” as used herein means a polynucleotide of genomic, cDNA, or synthetic origin or a combination thereof, which by virtue of its origin the “isolated polynucleotide” (1) is not associated with all or a portion of polynucleotides with which the “isolated polynucleotide” is found in nature, (2) is operably linked to a polynucleotide to which it is not linked in nature, or (3) does not occur in nature as part of a larger sequence.

The term “isolated protein”, “isolated polypeptide” or “isolated antibody” is a protein, polypeptide or antibody that by virtue of its origin or source of derivation: (1) is not associated with naturally associated components that accompany it in its native state; (2) is free of other proteins from the same species; (3) is expressed by a cell from a different species; or (4) does not occur in nature. Thus, a polypeptide that is, e.g., chemically synthesized or synthesized in a cellular system different from the cell from which it naturally originates will be “isolated” from its naturally associated components. A protein may also be rendered substantially free of naturally associated components by isolation, using protein purification techniques well known in the art.

Examples of isolated antibodies include a CD44 antibody that has been affinity purified using CD44, and a CD44 antibody that has been synthesized by a cell line in vitro.

“In vitro” refers to procedures performed in an artificial environment such as, e.g., without limitation, in a test tube or culture medium.

“In vivo” refers to procedures performed within a living organism such as, without limitation, a mammal, e.g. a monkey, mouse, rat or rabbit.

The term “K_{D}” refers to the binding affinity equilibrium constant of a particular antibody-antigen interaction. An antibody is said to specifically bind an antigen when the K_{D} is ≦1 mM, preferably ≦100 nM and most preferably ≦10 nM. A K_{D} binding affinity constant can be measured by surface plasmon resonance, for example using the BIACORE™ system as discussed in EXAMPLE 5.

The term “k_{off}” refers to the dissociation rate constant of a particular antibody-antigen interaction. A k_{off} dissociation rate constant can be measured by surface plasmon resonance, for example using the BIACORE™ system as discussed in EXAMPLE 5.

The term “naturally occurring nucleotides” as used herein includes deoxyribonucleotides and ribonucleotides. The term “modified nucleotides” as used herein includes, for example, nucleotides with modified or substituted sugar groups. The term “oligonucleotide linkages” referred to herein includes oligonucleotides linkages such as, for example, phosphorothioate, phosphorodithioate, phosphoroselenoate, phosphorodiselenoate, phosphoroanilothioate, phoshoraniladate, phosphoroamidate. LaPlanche et al., (1986) Nucl. Acids Res. 14:9081; Stec et al., (1984) J. Am. Chem. Soc. 106:6077; Stein et al., (1988) Nucl. Acids Res. 16:3209; Zon et al., (1991) Anti-Cancer Drug Design 6:539; Zon et al., Oligonucleotides and Analogues: A Practical Approach, pp. 87-108 (F. Eckstein, Ed., Oxford University Press, Oxford England (1991)); U.S. Pat. No. 5,151,510; Uhlmann and Peyman, (1990) Chemical Reviews 90:543. An oligonucleotide can include a label for detection, if desired.

“Operably linked” sequences include both expression control sequences that are contiguous with the gene of interest and expression control sequences that act in trans or at a distance to control the gene of interest.

The term “percent sequence identity” in the context of nucleic acid sequences means the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestht, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, (1990) Methods Enzymol. 183:63-98; Pearson, (2000) Methods Mol. Biol. 132:185-219; Pearson, (1996) Methods Enzymol. 266:227-258; Pearson, (1998) J. Mol. Biol. 276:71-84. Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1

A reference to a nucleotide sequence encompasses its complement unless otherwise specified. Thus, a reference to a nucleic acid having a particular sequence should be understood to encompass its complementary strand, with its complementary sequence.

The term “percent sequence identity” in the context of amino acid sequences means the residues in two sequences that are the same when aligned for maximum correspondence. The length of sequence identity comparison may be over a stretch of at least about five amino acids, usually at least about 20 amino acids, more usually at least about 30 amino acids, typically at least about 50 amino acids, more typically at least about 100 amino acids, and even more typically about 150, 200 or more amino acids. There are a number of different algorithms known in the art that can be used to measure amino acid sequence identity. For instance, amino acid sequences can be compared using FASTA, Gap or Bestfit, which are programs in Wisconsin Package Version 10.0, Genetics Computer Group (GCG), Madison, Wis.

Sequence identity for polypeptides is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters as specified by the programs to determine sequence homology or sequence identity between closely related polypeptides, such as homologous polypeptides from different species of organisms or between a wild type protein and an analog thereof. See, e.g., GCG Version 6.1 (University of Wisconsin, WI). Polypeptide sequences also can be compared using FASTA using default or recommended parameters, see GCG Version 6.1. FASTA (e.g., FASTA2 and FASTA3) provides alignments and percent sequence identity of the regions of the best overlap between the query and search sequences (Pearson, (1990) Methods Enzymol. 183:63-98; Pearson, (2000) Methods Mol. Biol. 132:185-219). Another preferred algorithm when comparing a sequence of the invention to a database containing a large number of sequences from different organisms is the computer program BLAST, especially blastp or tblastn, using default parameters as supplied with the programs. See, e.g., Altschul et al., (1990) J. Mol. Biol. 215:403-410; Altschul et al., (1997) Nucleic Acids Res. 25:3389-402.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

The term “polynucleotide” as referred to herein means a polymeric form of nucleotides of at least 10 bases in length, either ribonucleotides or deoxynucleotides or a modified form of either type of nucleotide. The term includes single and double stranded forms.

The term “polypeptide” encompasses native or artificial proteins, protein fragments and polypeptide analogs of a protein sequence. A polypeptide may be monomeric or polymeric.

The term “polypeptide fragment” as used herein refers to a polypeptide that has an amino-terminal and/or carboxy-terminal deletion, but where the remaining amino acid sequence is identical to the corresponding positions in the naturally-occurring sequence. In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “recombinant host cell” (or simply “host cell”), as used herein, means a cell into which a recombinant expression vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

A protein or polypeptide is “substantially pure,” “substantially homogeneous,” or “substantially purified” when at least about 60 to 75% of a sample exhibits a single species of polypeptide. The polypeptide or protein may be monomeric or multimeric. A substantially pure polypeptide or protein can typically comprise about 50%, 60%, 70%, 80% or 90% w/w of a protein sample, more usually about 95%, and preferably can be over 99% pure. Protein purity or homogeneity may be indicated by a number of means well known in the art, such as polyacrylamide gel electrophoresis of a protein sample followed by visualizing a single polypeptide band upon staining the gel with a stain well known in the art. As one skilled in the art will appreciate, higher resolution may be provided by using HPLC or other means well known in the art for purification.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, preferably at least about 90%, and more preferably at least about 95%, 96%, 97%, 98%, 99% or 100% of the nucleotide bases, as measured by any well-known algorithm of sequence identity, such as FASTA, BLAST or Gap, as discussed above.

As applied to polypeptides, the term “substantial identity” or “substantial similarity” means that two amino acid sequences, when optimally aligned, such as by the programs GAP or BESTFIT using default gap weights as supplied with the programs, share at least 70%, 75% or 80% sequence similarity, preferably at least 90% or 95% sequence identity, and more preferably at least 97%, 98%, 99% or 100% sequence identity. In certain embodiments, residue positions that are not identical differ by conservative amino acid substitutions.

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.). For further descriptions, see Jonsson U. et al., (1993) Ann. Biol. Clin. 51:19-26; Jonsson U. et al., (1991) Biotechniques 11:620-627; Jonsson B. et al., (1995) J. Mol. Recognit. 8:125-131; and Johnsson B. et al., (1991) Anal. Biochem. 198:268-277.

“Therapeutically effective amount” refers to that amount of the therapeutic agent being administered which will relieve to some extent one or more of the symptoms of the disorder being treated. In reference to the treatment of rheumatoid arthritis, a therapeutically effective amount refers to that amount which has at least one of the following effects: reducing the structural damage of joints; inhibiting (that is, slowing to some extent, preferably stopping) the accumulation of fluid in the joint area; and relieving to some extent (or, preferably, eliminating) one or more symptoms associated with rheumatoid arthritis.

“Treat”, “treating” and “treatment” refer to a method of alleviating or abrogating a biological disorder and/or its attendant symptoms. With regard to a variety of autoimmune disease, such as rheumatoid arthritis, atherosclerosis, granulomatous diseases and multiple sclerosis, these terms simply mean that the life expectancy of an individual affected with an autominnue disease will be increased or that one or more of the symptoms of the disease will be reduced.

As used herein, the term “utilizes” with reference to a particular gene means that the amino acid sequence of a particular region in an antibody was ultimately derived from that gene during B-cell maturation. For example, the phrase “a heavy chain variable region amino acid sequence that utilizes a human V_{H}-3 family gene” refers to the situation where the V_{H} region of the antibody was derived from the VH-3 family of gene segments during B-cell maturation. In human B-cells, there are more than 30 distinct functional heavy chain variable genes with which to generate antibodies. Use of a particular heavy chain variable gene, therefore, is indicative of a preferred binding motif of the antibody-antigen interaction with respect to the combined properties of binding to the antigen and functional activity. As will be appreciated, gene utilization analysis provides only a limited overview of antibody structure. As human B-cells stocastically generate V-D-J heavy or V-J kappa light chain transcripts, there are a number of secondary processes that occur, including, without limitation, somatic hypermutation, n-additions, and CDR3 extensions. See, for example, Mendez et al. Nature Genetics 15:146-156 (1997).

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)).

The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. In an embodiment, the vector is a viral vector, wherein additional DNA segments may be ligated into the viral genome. In an embodiment, the vectors are capable of autonomous replication in a host cell into which they are introduced (e.g., bacterial vectors having a bacterial origin of replication and episomal mammalian vectors). In other embodiment, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides, fluorescent labels (e.g., FITC, rhodamine, lanthanide phosphors), enzymatic labels, chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

The C-terminal lysine of the heavy chain of the anti CD44 antibody of the invention may be cleaved when the antibody is recombinantly produced as a result the activitity of one or more carboxypeptidases when the antibody is expressed in mammalian cell culture (Lewis D. A., et al., Anal. Chem., 66(5): 585-95 (1994); Harris R. J., J. of Chromotography A, 705: 129-134 (1995)). A number of variations from the expected structure may be found in recombinantly produced antibodies resulting from either known or novel types of in vivo (posttranslational) modification or from spontaneous (nonenzymatic) protein degradation, such as methionine oxidation, diketopiperazine formation, aspartate isomerization and deamidation of asparagine residues, or succinimide formation.

### Human Anti-CD44 Antibodies and Characterization Thereof

This invention provides isolated human antibodies, or antigen-binding portions thereof, that bind to human CD44. Various aspects of the invention relate to such antibodies and antigen-binding portions, and pharmaceutical compositions thereof, as well as nucleic acids, recombinant expression vectors and host cells for making such antibodies and antigen-binding portions. Methods of using the antibodies and antigen-binding portions of the present invention to detect human CD44 or to inhibit human CD44 activity, either in vitro or in vivo, are also encompassed by the invention. Human anti-CD44 antibodies according to preferred embodiments of the present invention minimize the immunogenic and allergic responses intrinsic to non-human or non-human-derivatized monoclonal antibodies (Mabs) and thus increase the efficacy and safety of the administered antibodies. The use of fully human antibodies provides a substantial advantage in the treatment of chronic and recurring human diseases, such as rheumatoid arthritis, Juvenile Rheumatoid Arthritis, atherosclerosis, granulmatous diseases, multiples sclerosis, asthma, Crohn's Disease, Ankylosing Spondylitis, Psoriatic Arthritis, Plaque Psoriasis and cancer, which may require repeated antibody administrations.

The CD44 amino acid and nucleotide sequences from several species, including human, are known, SEQ ID NO: 1 and 2 (see, e.g., Accession No. NM_{—}001001391). Human CD44, or antigenic portions thereof, can be prepared according to methods well known to those in the art, or can be purchased from commercial vendors (for e.g., from R&D Systems Cat. No. 861-PC-100). The CD44 amino acid and nucleotide sequences from cynomolgus monkey, are not known in the art and are disclosed herein, SEQ ID NOs: 5, 7 (amino acid), 8 and 153 (nucleic acid).

In some embodiments, human anti-CD44 antibodies are produced by immunizing a non-human transgenic animal, e.g., a rodent, whose genome comprises human immunoglobulin genes so that the transgenic animal produces human antibodies. In some embodiments, the anti-CD44 antibodies and antigen-binding portions include, but are not limited to, antibodies or antigen-binding portions which bind to the HA binding site.

In a further embodiment, the present invention provides an antibody or antigen-binding portion thereof, wherein said antibody or antigen-binding portion comprises at least one CDR selected from: a V_{H} CDR1 that is independently selected from any one of SEQ ID NOs: 17, 53, 89 and 125 or a sequence that differs from any one of SEQ ID NOs: 17, 53, 89 and 125 by at least one conservative amino acid substitution; a V_{H} CDR2 that is independently selected from any one of SEQ ID NOs:19, 55, 91 and 127 or a sequence that differs from any one of SEQ ID NOs: 19, 55, 91 and 127 by at to least one conservative amino acid substitution; and a V_{H} CDR3 that is independently selected from any one of SEQ ID NOs:21, 57, 93 and 129 or a sequence that differs from any one of SEQ ID NOs: 21, 57, 93 and 129 by at least one conservative amino acid substitution. For example, the V_{H} CDR1, CDR2, and CDR3 sequences mentioned above can each independently differ from the respective recited SEQ ID NOs by 1, 2, 3, 4 or 5 conservative amino acid substitutions.

In another embodiment, the present invention provides an antibody or antigen-binding portion thereof, wherein said antibody or antigen-binding portion comprises at least one CDR selected from: a V_{L} CDR1 that is independently selected from any one of SEQ ID NOs: 23, 59, 95 and 131 or a sequence that differs from any one of SEQ ID NOs: 23, 59, 95 and 131 by at least one conservative amino acid substitution; a V_{L} CDR2 that is independently selected from any one of SEQ ID NOs:25, 61, 97 and 133 or a sequence that differs from any one of SEQ ID NOs: 25, 61, 97 and 133 by at least one conservative amino acid substitution; and a V_{L} CDR3 that is independently selected from any one of SEQ ID NOs:27, 63, 99 and 137 or a sequence that differs from any one of SEQ ID NOs: 27, 63, 99 and 135 by at least one conservative amino acid substitution. For example, the V_{L} CDR1, CDR2, and CDR3 sequences mentioned above can each independently differ from the respective recited SEQ ID NOs by 1, 2, 3, 4 or 5 conservative amino acid substitutions.

In yet a further aspect of the present invention an antibody or antigen-binding portion comprises: a V_{H} CDR1 as set forth in SEQ ID NO:17, a V_{H} CDR2 as set forth in SEQ ID NO:19, a V_{H} CDR3 as set forth in SEQ ID NO:21, a V_{L} CDR1 as set forth in SEQ ID NO:23, a Vₜ. CDR2 as set forth in SEQ ID NO:25, and a V_{L} CDR3 as set forth in SEQ ID NO:27.

In yet a further aspect of the present invention an antibody or antigen-binding portion comprises: a V_{H} CDR1 as set forth in SEQ ID NO:53, a V_{H} CDR2 as set forth in SEQ ID NO:55, a V_{H} CDR3 as set forth in SEQ ID NO:57, a V_{L} CDR1 as set forth in SEQ ID NO:59, a V_{L} CDR2 as set forth in SEQ ID NO:61, and a V_{L} CDR3 as set forth in SEQ ID NO:63.

In yet a further aspect of the present invention an antibody or antigen-binding Portion comprises: a V_{H} CDR1 as set forth in SEQ ID NO:89, a V_{H} CDR2 as set forth in SEQ ID NO:91, a V_{H} CDR3 as set forth in SEQ ID NO:93, a V_{L} CDR1 as set forth in SEQ ID NO:95, a V_{L} CDR2 as set forth in SEQ ID NO:97, and a V_{L} CDR3 as set forth in SEQ ID NO:99.

In yet another aspect of the present invention an antibody or antigen-binding portion comprises: a V_{H} CDR1 as set forth in SEQ ID NO:125, a V_{H} CDR2 as set forth in SEQ ID NO:127, a V_{H} CDR3 as set forth in SEQ ID NO:129, a V_{L} CDR1 as set forth in SEQ ID NO:131, a V_{L} CDR2 as set forth in SEQ ID NO:133, and a V_{L} CDR3 as set forth in SEQ ID NO:135

In a further embodiment, the V_{H} and V_{L} CDR1, CDR2, and CDR3 sequences mentioned above can also each independently differ from the specific SEQ ID NOs recited above by at least one conservative amino acid substitution. For example, the CDR1, CDR2, and CDR3 sequences can each independently differ by 1, 2, 3, 4, or 5 conservative amino acid substitutions from the respective specific SEQ ID NOs recited above.

The present invention further provides an antibody or antigen-binding portion thereof wherein said antibody or antigen-binding portion comprises the V_{H} and V_{L} CDR1, the V_{H} and V_{L} CDR2, and the V_{H} and V_{L} CDR3 as found in any one of antibodies 1A9.A6.B9; 2D1.A3.D12; 14G9.B8.B4 and 10C8.2.3.

In a further embodiment, the antibody or antigen-binding portion thereof comprising a V_{H} domain that is any of SEQ ID NOs: 11, 47, 83 and 119, or differs from any one of SEQ ID NOs: 11, 47, 83 and 119 by having at least one conservative amino acid substitution. For example, the V_{H} domain can differ by 1, 2, 3, 4, 5, 6, 7, 8, 9, or 10 conservative amino acid substitutions from any of SEQ ID NOs: 11, 47, 83 and 119. In a further embodiment, any of these conservative amino acid substitutions can occur in the CDR1, CDR2, and/or CDR3 regions.

A further aspect of the present invention is an antibody or antigen-binding portion thereof comprising a V_{H} domain that is at least 90%, preferably 95%, and more preferably 96%, 97%, 98%, 99% or 100% identical in amino acid sequence to any of SEQ ID NOs: 11, 47, 83 and 119.

In a further embodiment, the antibody or antigen-binding portion thereof comprises a V_{L} domain that is any of SEQ ID NOs: 15, 51, 87 and 123, or differs from any of SEQ ID Nos: 15, 51, 87 and 123 by having at least one conservative amino acid substitution. For example, the V_{L} domain can differ by 1, 2, 3, 4, 5, 6, 7, 8, 9, or conservative amino acid substitutions from any of SEQ ID NOs: 15, 51, 87 and 123. In a further embodiment, any of these conservative amino acid substitutions can occur in the CDR1, CDR2, and/or CDR3 regions.

A further aspect of the present invention is an antibody or antigen-binding portion thereof comprising a V_{L} domain that is at least 90%, preferably 95%, and more preferably 96%, 97%, 98%, 99% or 100% identical in amino acid sequence to any of SEQ ID NOs: 15, 51, 87 and 123.

In another aspect of the present invention the antibody or antigen-binding portion thereof is selected from the group consisting of: a) an antibody or antigen-binding portion thereof that comprises a V_{H} domain as set forth in SEQ ID NO:11, and a V_{L} domain as set forth in SEQ ID NO:15; b) an antibody or antigen-binding portion thereof that comprises a V_{H} domain as set forth in SEQ ID NO:47, and a V_{L} domain as set forth in SEQ ID NO:51; c) an antibody or antigen-binding portion thereof that comprises a V_{H} domain as set forth in SEQ ID NO:83 and a V_{L} domain as set forth in SEQ ID NO:87; and d) an antibody or antigen-binding portion thereof that comprises a V_{H} domain as set forth in SEQ ID NO:119 and a V_{L} domain as set forth in SEQ ID NO:123.

In a further embodiment, for any of the antibodies or antigen-binding portions thereof as described above in groups a) to d) the V_{H} and/or V_{L} domains can differ from the specific SEQ ID NOs recited therein by at least one conservative amino acid substitution. For example, the V_{H} and/or V_{L} domains can differ from the recited SEQ ID NO by 1, 2, 3, 4, 5, 6, 7, 8, 9, or 10 conservative amino acid substitutions. In a further embodiment, any of these conservative amino acid substitutions can occur in the CDR1, CDR2, and/or CDR3 regions.

In yet another aspect, the present invention is an antibody or antigen-binding portion thereof that is selected from the group consisting of: a) an antibody or antigen-binding portion thereof that comprises a heavy chain that is at least 90%, preferably 95%, and more preferably 96%, 97%, 98%, 99% or 100% identical in amino acid sequence to SEQ ID NO:9, and a light chain that is at least 90%, preferably 95%, 96%, 97%, 98%, 99% or 100% identical in amino acid sequence to SEQ ID NO:13; b) an antibody or antigen-binding portion thereof that comprises a heavy chain that is at least 90% identical in amino acid sequence to SEQ ID NO:45, and a light chain that is (at least 95% preferably 96%, 97%, 98%, 99% or 100% identical to SEQ ID NO:49; c) an antibody or antigen-binding portion thereof that comprises a heavy chain that is at least 95%, more preferably 96%, 97%, 98%, 99% or 100% identical to SEQ ID NO:81, and a light chain that is 95%, preferably 96%, 97%, 98%, 99% or 100% identical to SEQ ID NO:85; and d) an antibody or antigen-binding portion thereof that comprises a heavy chain that is at least 90% identical to SEQ ID NO:117, and a light chain that is preferably 95%, more preferably 96%, 97%, 98%, 99% or 100% identical to SEQ ID NO:121.

In another embodiment, the present invention is an antibody or antigen-binding portion thereof that is selected from the group consisting of: a) an antibody or antigen-binding portion thereof that comprises a heavy chain as set forth in SEQ ID NO:9, and a light chain as set forth in SEQ ID NO:13; b) an antibody or antigen-binding portion thereof that comprises a heavy chain as set forth in SEQ ID NO:45 and a light chain as set forth in SEQ ID NO:49; c) an antibody or antigen-binding portion thereof that comprises a heavy chain as set forth in SEQ ID NO:81, and a light chain as set forth in SEQ ID NO:85; and d) an antibody or antigen-binding portion thereof that comprises a heavy chain that is set forth in SEQ ID NO:117, and a light chain that is set forth in SEQ ID NO:121.

In some embodiments, the C-terminal lysine of the heavy chain of the anti CD44 antibody of the invention is cleaved (Lewis D. A., et al., Anal. Chem., 66(5): 585-95 (1994); Harris R. J., J. of Chromotography, 705: 129-134 (1995)).

In various embodiments of the invention, the heavy and/or light chain(s) of the anti-CD44 antibodies or antigen binding portion thereof may optionally include a signal sequence.

The invention further provides CD44 antibodies or antigen-binding portions thereof wherein the antibody or antigen-binding portion thereof, or CDR(s) thereof as described having at least one of several functional properties as described below in A) thru G).

A) For example, in one embodiment, the antibodies or antigen-binding portions thereof bind to CD44 with a K_{D} of 1000 nM or less as measured by surface plasmon resonance. In a further embodiment, the antibody or portion binds to CD44 with a K_{D} of less than 500 nM or preferably, less than 100 nM, less than 50 nM, less than 20 nM, less than 10 nM, less than 5 nM, less than 4 nM, less than 3 nM, less than 2 nM, less than 1 nM, less than 900 pM, less than 800 pM, less than 700 pM, less than 600 pM, less than 500 pM, or less than 100 pM, as measured by surface plasmon resonance. Typically, there is no lower limit on the value of K_{D}. For practical purposes, however, the lower limit can be assumed to be about 1 pM.

B) In another embodiment, the antibodies or antibody-binding portions thereof have an off rate (k_{off}) for CD44 of less than or equal to 0.01^{s-1} as measured by surface plasmon resonance. For example, in certain embodiments the antibody or portion has a k_{off} for CD44 of less than 0.005^{s-1}, less than 0.004^{s-1}, less than 0.003^{s-1}, less than 0.002^{s-1}, or less than 0.001^{s-1}. Typically, there is no lower limit for the value of k_{off}. For practical purposes, however, the lower limit can be assumed to be about 1×10^{-7 s-1}.

C) In a further embodiment the antibodies or antigen-binding portions thereof bind to CD44 with an EC₅₀ of less than 500 nM, 75 μg/ml as measured by FACS or ELISA binding assay. In a further embodiment, the antibody or portion binds to CD44 with an EC₅₀ of less than 100 nM, less than 50 nM, less than 20 nM, less than 10 nM, less than 1 nM, less than 500 pM, or less than 100 pM, as measured by ELISA. Preferably, the antibody or portion binds to CD44 with an EC₅₀ of less than 10 nM, 1.5 μg/ml. Typically, there is no lower limit on the value of EC₅₀. For practical purposes, however, the lower limit can be assumed to be about 1 pM.

D) In still another embodiment, the antibodies or antigen-binding portions thereof inhibit the interaction between CD44 and HA with an IC₅₀ of less than 500 nM, 75 μg/ml as measured by an ELISA binding assay. In a further embodiment, the antibody or portion binds to CD44 with an IC₅₀ of less than 100 nM, less than 50 nM, less than 20 nM, less than 10 nM, less than 5 nM, less than 4 nM, less than 3 nM, less than 2 nM, less than 1 nM, less than 500 pM, or less than 100 pM, as measured by an ELISA binding assay.

E) In still another embodiment, the antibodies or antigen-binding portions thereof reduce the in vivo surface express and monocytes and at an IC₅₀ of less than about 100 nM, as measured by FACS.

F) In another embodiment, the antibodies or antigen-binding portions reduce the surface expression of CD44 receptors in vitro with an IC₅₀ of less than 50 nM, less than 20 nM, less than 10 nM, less than 1 nM, less than 500 pM, or less than 100 pM, less than about 20 nM, less than about 10 nM, or less than about 5 nM) as measured by FACS. Preferably, the antibody or antigen-binding portion reduces the surface expression of CD44 receptors with an IC₅₀ of less than 30 nM, 4.5 μg/ml. For practical purposes, however, the lower limit can be assumed to be about 1 pM.

G) In another embodiment, the anti-CD44 antibody or antigen-binding portions thereof has selectivity for CD44 over lymphatic vessel endothelial hyauronan receptor 1 protein (LYVE-1) by at least 100 fold.

In one embodiment, the invention provides human anti-CD44 monoclonal antibodies (mAbs), designated as: 1A9.A6.B9; 2D1.A3.D12; 14G9.B8.B4; and 10C8.2.3; and the hybridoma cell lines that produce them. TABLES 1 and 9-12 of the application shows the sequence identifiers (SEQ ID NOs:) of the nucleic acids encoding the full-length heavy and light chains, the corresponding full-length deduced amino acid sequences, and the nucleotide and deduced amino acid sequences of the heavy and light chain variable regions.

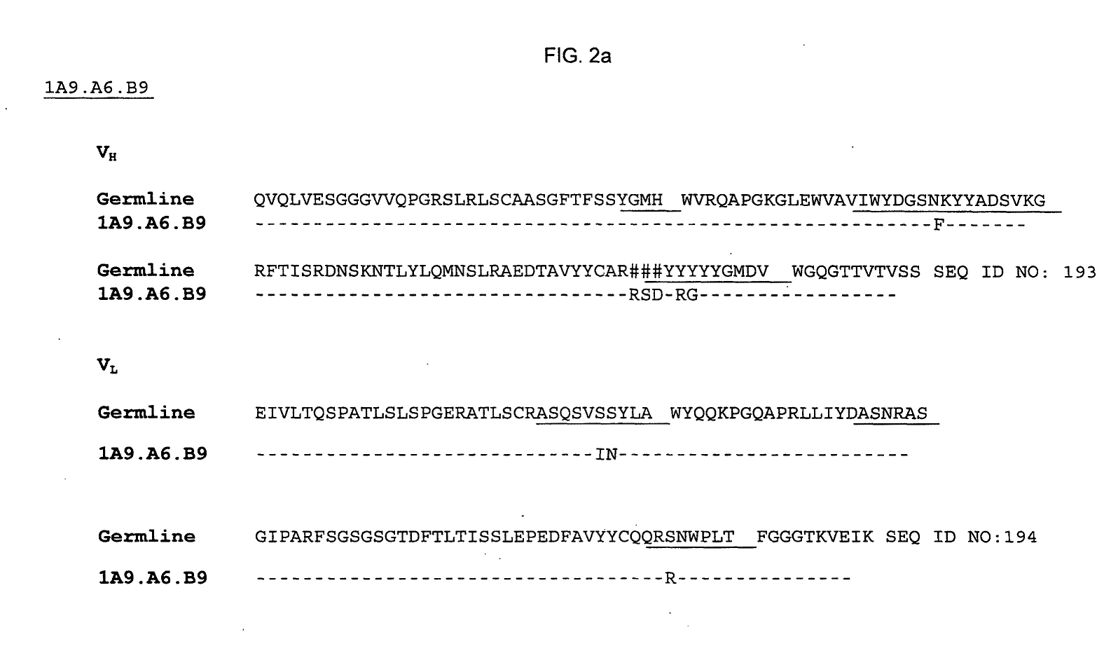

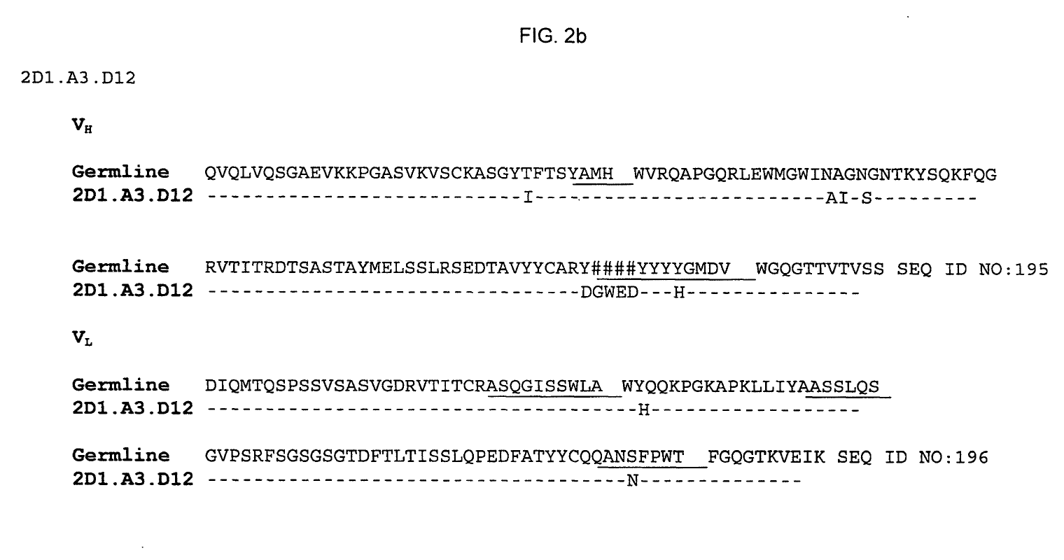

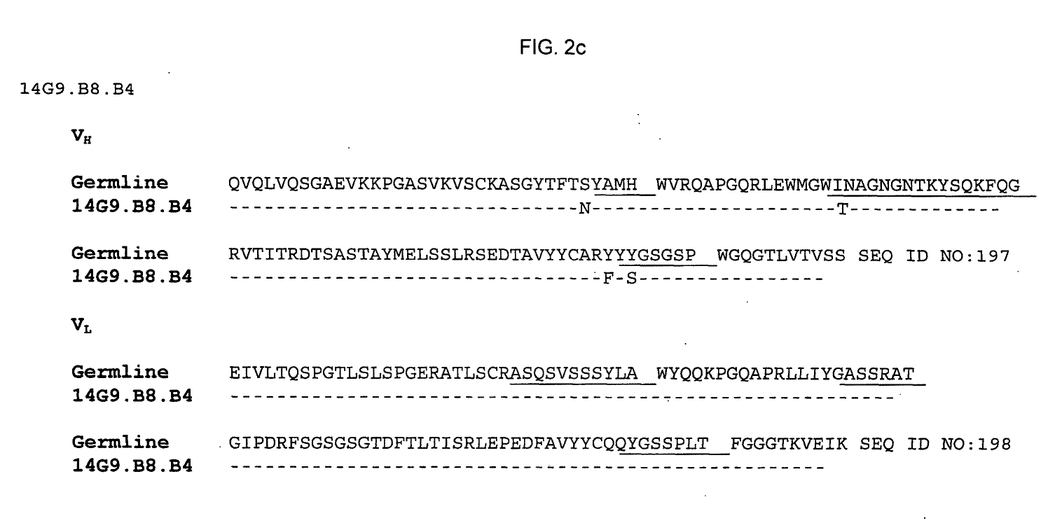

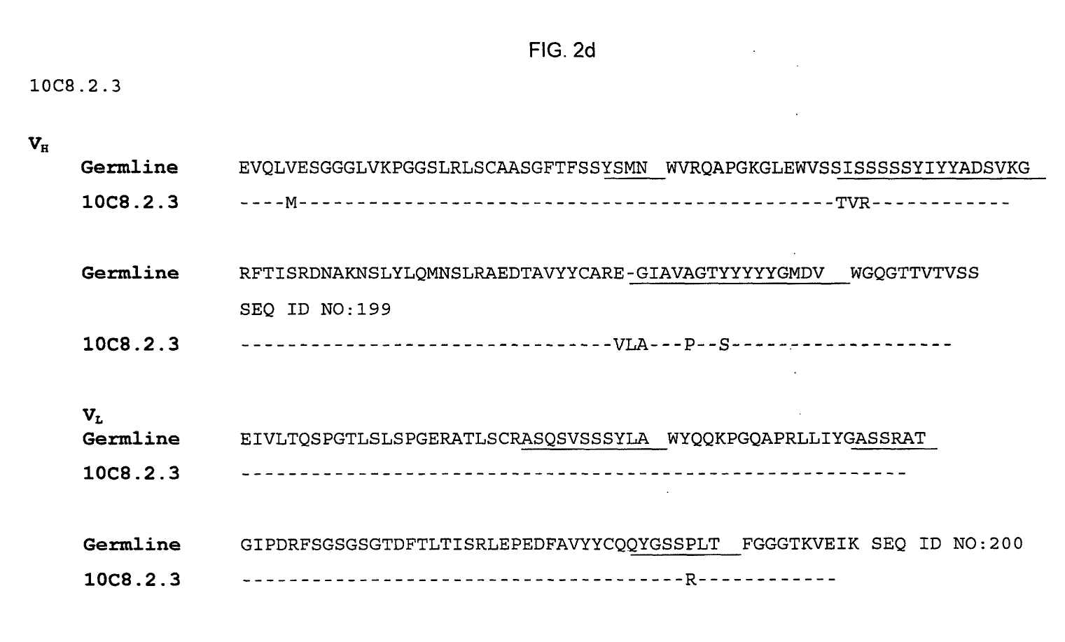

In embodiments, antibodies are IgGs designated as: 1A9.A6.B9; 2D1.A3.D12; 14G9.B8.B4; and 10C8.2.3. The specific amino acid sequences of the antibodies or antigen-binding portions thereof or antibody domains of the present invention are described in Tables 9, 10, 11 & 12, and FIG. 2.

In an embodiment modification, the V_{L} of the CD44 antibody comprise one or more amino acid substitutions relative to the germline amino acid sequence of the human gene. In some embodiments, the V_{L} of the anti-CD44 antibody comprises 1, 2, 3, 4, 5, 6, 7, 8, 9, or 10 amino acid substitutions relative to the germline amino acid sequence. In an embodiment, one or more of those substitutions from germline is in the CDR regions of the light chain. In an embodiment, the amino acid substitutions relative to germline are at one or more of the same positions as the substitutions relative to germline in any one or more of the V_{L} of antibodies 1A9.A6.B9; 2D1.A3.D12; 14G9.B8.B4; and 10C8.2.3. For example, the V_{L} of an anti-CD44 antibody of the invention may contain one or more amino acid substitutions compared to germline found in the V_{L} of antibody 1A9.A6.B9. In some embodiments, the amino acid changes are at one or more of the same positions, but involve a different substitution than in the reference antibody.

In an embodiment, amino acid changes relative to germline occur at one or more of the same positions as in any of the V_{L} of antibodies 1A9.A6.B9; 2D1.A3.D12; 14G9.B8.B4 and 10C8.2.3, but the changes may represent conservative amino acid substitutions at such position(s) relative to the amino acid in the reference antibody. For example, if a particular position in one of these antibodies is changed relative to germline and is glutamate, one may substitute aspartate at that position. Similarly, if an amino acid substitution compared to germline is serine, one may conservatively substitute threonine for serine at that position. Conservative amino acid substitutions are discussed supra.

In some embodiments, the light chain of the human anti-CD44 antibody comprises the V_{L} amino acid sequence of antibody 1A9.A6.B9 (SEQ ID NO:15); 2D1.A3.D12 (SEQ ID NO:51); 14G9.B8.B4 (SEQ ID NO:87) or 10C8.2.3 (SEQ ID NO:123) or said amino acid sequence having up to 1, 2, 3, 4, 5, 6, 7, 8, 9, or 10 conservative amino acid substitutions and/or a total of up to 3 non-conservative amino acid substitutions. In some embodiments, the light chain comprises the amino acid sequence from the beginning of the CDR1 to the end of the CDR3 of any one of the foregoing antibodies.

In some embodiments, the light chain may comprise CDR1, CDR2 and CDR3 regions independently selected from the light chain CDR1, CDR2 and CDR3, respectively, of the light chain of antibodies 1A9.A6.B9; 2D1.A3.D12; 14G9.B8.B4 and 10C8.2.3, or CDR regions each having less than 4 or less than 3 conservative amino acid substitutions and/or a total of three or fewer non-conservative amino acid substitutions. In some embodiments, the light chain of the anti-CD44 antibody comprises a light chain CDR1, CDR2, and CDR3, each of which are independently selected from the light chain CDR1, CDR2 and CDR3 regions of monoclonal antibody 1A9.A6.B9 (SEQ ID NO:13); 2D1.A3.D12 (SEQ ID NO:49); 14G9.B8.B4 (SEQ ID NO:85) or 10C8.2.3 (SEQ ID NO:121). In certain embodiments, the light chain of the anti-CD44 antibody comprises the light chain CDR1, CDR2 and CDR3 regions of an antibody comprising the amino acid sequence of the V_{L} region of an antibody selected from 1A9.A6.B9 (SEQ ID NO:15); 2D1.A3.D12 (SEQ ID NO:51); 14G9.B8.B4 (SEQ ID NO:87) or 10C8.2.3 (SEQ ID NO:123) or said CDR regions each having less than 4 or less than 3 conservative amino acid substitutions and/or a total of three or fewer non-conservative amino acid substitutions.

An anti-CD44 antibody of the invention can comprise a human kappa or a human lambda light chain or an amino acid sequence derived therefrom. In some embodiments comprising a kappa light chain, the light chain variable domain (V_{L}) is encoded in part by a human V_{K}1, V_{K}2 or V_{K}3 family gene. In certain embodiments, the light chain utilizes a human, or monkey amino acid sequence or combination thereof.

With regard to the heavy chains, in an embodiment, the variable domains (V_{H}) is encoded, in part, by a human gene. In some embodiments, the V_{H} sequence of the anti-CD44 antibody contains one or more amino acid substitutions, deletions or insertions (additions) relative to the germline amino acid sequence. In some embodiments, the variable domain of the heavy chain comprises 1, 2, 3, 4, 5, 6, 7, 8, 9, 10, 11, 12, 13, 14, 15, 16, or 17 mutations from the germline amino acid sequence. In some embodiments, the mutation(s) are non-conservative substitutions, deletions or insertions, compared to the germline amino acid sequence. In some embodiments, the mutations are in the CDR regions of the heavy chain. In some embodiments, the amino acid changes are made at one or more of the same positions as the mutations from germline in any one or more of the V_{H} of antibodies 1A9.A6.B9; 2D1.A3.D12; 14G9.B8.B4 or 10C8.2.3. In other embodiments, the amino acid changes are at one or more of the same positions but involve a different mutation than in the reference antibody.

In some embodiments, the heavy chain comprises the V_{H} amino acid sequence of antibody 1A9.A6.B9 (SEQ ID NO:11); 2D1.A3.D12 (SEQ ID NO:47); 14G9.B8.B4 (SEQ ID NO:83) or 10C8.2.3 (SEQ ID NO:119) said V_{H} amino acid sequence having up to 1, 2, 3, 4, 6, 8, or 10 conservative amino acid substitutions and/or a total of up to 3 non-conservative amino acid substitutions. In some embodiments, the heavy chain comprises the amino acid sequence from the beginning of the CDR1 to the end of the CDR3 of any one of the foregoing antibodies.

In an embodiment, the heavy chain comprises the heavy chain CDR1, CDR2 and CDR3 regions of antibody 1A9.A6.B9; 2D1.A3.D12; 14G9.B8.B4 or 10C8.2.3 or said CDR regions each having less than 8, less than 6, less than 4, or less than 3 conservative amino acid substitutions and/or a total of three or fewer non-conservative amino acid substitutions. In some embodiments, the heavy chain CDR regions are independently selected from the CDR regions of antibodies 1A9.A6.B9; 2D1.A3.D12; 14G9.B8.B4 or 10C8.2.3. In another embodiment, the heavy chain comprises CDR regions independently selected from two or more V_{H} regions selected from 1A9.A6.B9 (SEQ ID NO:11); 2D1.A3.D12 (SEQ ID NO:47); 14G9.B8.B4 (SEQ ID NO:83) or 10C8.2.3 (SEQ ID NO:119).

In another embodiment, the antibody comprises a light chain and a heavy chain. In a further embodiment, the light chain CDRs and the heavy chain CDRs are from the same antibody.

One type of amino acid substitution that may be made is to change one or more cysteines in the antibody, which may be chemically reactive, to another residue, such as, without limitation, alanine or serine. In one embodiment, there is a substitution of a non-canonical cysteine. The substitution can be made in a CDR or framework region of a variable domain or in the constant domain of an antibody. In some embodiments, the cysteine is canonical.

Another type of amino acid substitution that may be made is to change any potential proteolytic sites in the antibody. Such sites may occur in a CDR or framework region of a variable domain or in the constant domain of an antibody. Substitution of cysteine residues and removal of proteolytic sites may decrease the risk of any heterogeneity in the antibody product and thus increase its homogeneity. Another type of amino acid substitution is to eliminate asparagine-glycine pairs, which form potential deamidation sites, by altering one or both of the residues.

In embodiments of the invention, the heavy and light chains of the anti-CD44 antibodies may optionally include a signal sequence.

In one aspect, the invention provides four preferred inhibitory human anti-CD44 monoclonal antibodies and the hybridoma cell lines that produce them. TABLE 1 lists the sequence identifiers (SEQ ID NOs:) of the nucleic acids encoding the full-length and variable domain-comprising portions of heavy and light chains, and the corresponding or deduced amino acid sequences.

TABLE 1
HUMAN ANTI-CD44 ANTIBODIES

| | SEQUENCE IDENTIFIER (SEQ ID NO:) | | | | | | | |
|---|---|---|---|---|---|---|---|---|
| | Variable Domain Comprising Portion | | | | Full Length | | | |
| Monoclonal | Heavy | | Light | | Heavy | | Light | |
| Antibody | Protein | DNA | Protein | DNA | Protein | DNA | Protein | DNA |
| 1A9.A6.B9 | 11 | 12 | 15 | 16 | 9 | 10 | 13 | 14 |
| 2D1.A3.D12 | 47 | 48 | 51 | 52 | 45 | 46 | 49 | 50 |
| 14G9.B8.B4 | 83 | 84 | 87 | 88 | 81 | 82 | 85 | 86 |
| 10C8.2.3 | 119 | 120 | 123 | 124 | 117 | 118 | 121 | 122 |

In some embodiments, the invention provides heavy and light chain variants of monoclonal antibodies 1A9.A6.B9; 2D1.A3.D12; 14G9.B8.B4 or 10C8.2.3. As discussed in greater detail in EXAMPLE 3 of the present invention, numerous heavy and light chain variant mutations were made to match those in the germline CDR regions. For example in one embodiment of the present invention, g-1A9.A6.B9, g-2D1.A3.D12, g-14G9.B8.B4 and g-10C8.2.3 are the germlined versions of 1A9.A6.B9, 2D1.A3.D12, 14G9.B8.B4, and 10C8.2.3, respectively. The specific amino acids that were mutated to arrive at the germlined versions are apparent to those of skill in the art by comparing the sequences of the germlined vs. a non-germlined antibody. For example, the invention provides one amino acid substitution in the heavy chain of antibody 2D1.A3.D12, wherein threonine at residue 28 is changed to an isoleucine. A second point mutation is in the light chain of antibody 2D1.A3.D12, and substitutes the glutamine at residue 38 with a histidine.

As will be appreciated, gene utilization analysis provides only a limited overview of antibody structure. As human B-cells stocastically generate V-D-J heavy or V-J kappa light chain transcripts, there are a number of secondary processes that occur, including, without limitation, somatic hypermutation, n-additions, and CDR3 extensions. See, for example, Mendez et al., (1997) Nature Genetics 15:146-156 and U.S. Publication Patent Application No. 2003-0070185. Accordingly, to further examine antibody structures of the present invention, predicted amino acid sequences of the antibodies were generated from the cDNAs obtained from the clones. In addition, N-terminal amino acid sequences were obtained through protein sequencing. TABLE 2 below illustrates the germline gene segment usage and isotypes of the four anti-CD44 hybridoma derived antibodies.

TABLE 2

| | Heavy chain | | | Light chain | | |
|---|---|---|---|---|---|---|
| Clone | V_{H} | D | J_{H} | V_{L} | J_{K} | Isotype |
| 1A9.A6.B9 | 3-33 | D4-17 | JH6b | L6 | JK4 | IgG2 |
| 2D1.A3.D12 | 1-03 | nd | JH6b | L19 | JK1 | IgG1 |
| 14G9.B8.B4 | 1-03 | D3-10 | JH5b | A27 | JK4 | IgG1 |
| 10C8.2.3 | 3-21 | D6-19 | JH6b | A27 | JK4 | IgG4 |

nd = not determined

In an alternate embodiment, the invention relates to an antibody or antigen binding portion thereof that specifically binds to human CD44 and has a V_{H} and V_{L} gene utilization selected from the group consisting of 1) V_{H} D4-17 and V_{L}L6; 2) V_{H} D3-10 and V_{L}A27; and 3) V_{H} D6-19 and V_{L}A27.

Another embodiment provides any of the antibodies or antigen-binding portions described above which is an Fab fragment, an F(ab′)₂ fragment, an Fᵥ fragment, a single chain Fv fragment, a single chain V_{H} fragment, a single chain V_{L} fragment, a humanized antibody, a chimeric antibody or a bispecific antibody.

In a further embodiment there is provided a derivatized antibody or antigen-binding portion comprising any of the antibodies or portions thereof as described herein and at least one additional molecular entity. For example, the at least one additional molecular entity can be another antibody (e.g., a bispecific antibody or a diabody), a detection agent, a label, a cytotoxic agent, a pharmaceutical agent, and/or a protein or peptide that can mediate association of the antibody or antigen-binding portion with another molecule (such as a streptavidin core region or a polyhistidine tag) and/or a carrier protein (e.g. a blood protein albumin or transferrin) linked or fused (fusion protein) to the antibody or antigen-binding portion. For example, useful detection agents with which an antibody or antigen-binding portion of the invention may be derivatized include fluorescent compounds; inter alia, fluorescein, fluorescein isothiocyanate, rhodamine, 5-dimethylamine-1-naphthalenesulfonyl chloride, phycoerythrin, lanthanide phosphors. An antibody can also be labeled with enzymes that are useful for detection, such as, for example, horseradish peroxidase, β-galactosidase, luciferase, alkaline phosphatase, glucose oxidase. In a further embodiment the antibodies or antigen-binding portions thereof of the present invention can also be labeled with biotin, or with a predetermined polypeptide epitope recognized by a secondary reporter (e.g., leucine zipper pair sequences, binding sites for secondary antibodies, metal binding domains, epitope tags). In a still further embodiment of the present invention, any of the antibodies or antigen-binding portions thereof can also be derivatized with a chemical group such as polyethylene glycol (PEG), a methyl or ethyl group, or a carbohydrate group.

In some embodiments, the CD44 antibodies or antigen binding portions disclosed herein are attached to a solid support or particle. Such particles may be used for in vivo or in vitro diagnostic uses.

### Class and Subclass of Anti-CD44 Antibodies

The class (e.g., IgG, IgM, IgE, IgA, or IgD) and subclass (e.g. IgG1, IgG2, IgG3, or IgG4) of CD44 antibodies may be determined by any method known in the art. In general, the class and subclass of an antibody may be determined using antibodies that are specific for a particular class and subclass of antibody. Such antibodies are commercially available. The class and subclass can be determined by ELISA, or Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies. The CD44 antibodies of the present invention can be an IgG, an IgM, an IgE, an IgA, or an IgD molecule. For example, the CD44 antibodies can be an IgG that is an IgG1, IgG2, IgG3, or an IgG4 subclass.

One aspect of the invention provides a method for converting the class or subclass of a CD44 antibody to another class or subclass. In some embodiments, a nucleic acid molecule encoding a V_{L} or V_{H} that does not include sequences encoding C_{L} or C_{H} is isolated using methods well-known in the art. The nucleic acid molecule then is operatively linked to a nucleic acid sequence encoding a C_{L} or C_{H} from a desired immunoglobulin class or subclass. This can be achieved using a vector or nucleic acid molecule that comprises a C_{L} or C_{H} chain, as described above. For example, a CD44 antibody that was originally IgM can be class switched to an IgG. Further, the class switching may be used to convert one IgG subclass to another, e.g., from IgG1 to IgG2. Another method for producing an antibody of the invention comprising a desired isotype comprises the steps of isolating a nucleic acid encoding a heavy chain of a CD44 antibody and a nucleic acid encoding a light chain of a CD44 antibody, isolating the sequence encoding the V_{H} region, ligating the V_{H} sequence to a sequence encoding a heavy chain constant domain of the desired isotype, expressing the light chain gene and the heavy chain construct in a cell, and collecting the CD44 antibody with the desired isotype.

### Species and Molecular Selectivity

In another aspect of the invention, the anti-CD44 antibodies demonstrate both species and molecular selectivity. In some embodiments, the anti-CD44 antibody binds to human and primate CD44. Preferably the anti-CD44 binds to human, and cynomolgus monkey CD44. Following the teachings of the specification, one may determine the species selectivity for the anti-CD44 antibody using methods well known in the art. For instance, one may determine the species selectivity using a Western blot, flow cytometry, an ELISA, an immunoprecipitation or a RIA. (See, e.g., EXAMPLE 5).

In another embodiment, the anti-CD44 antibody has a selectivity for CD44 over lymphatic vessel endothelial hyauronan receptor 1 protein (LYVE-1) by at least 100 fold. (See EXAMPLE 11) One can determine the selectivity of the anti-CD44 antibody for CD44 using methods well known in the art following the teachings of the specification. For instance, one can determine the selectivity using a Western blot, flow cytometry, an ELISA, an immunoprecipitation or a RIA.

### Binding Affinity of Anti-CD44 Antibodies to CD44

In an embodiment, the anti-CD44 antibody binds to mammalian CD44 preferably human with high affinity.

In an embodiment, the anti-CD44 antibody binds within the HA binding domain.

In another embodiment, the anti-CD44 antibodies bind with high affinity to a polypeptide consisting of the amino acid sequence set forth in SEQ ID NO:3 (extracellular domain IgG fusion) or in SEQ ID NO:154 (monkey extracellular IgG fusion), and preferably binds with high affinity to a polypeptide consisting of the amino acid sequence of the HA binding domain.

In another embodiment, the anti-CD44 antibody binds to CD44, or more preferably to the HA binding domain, with a K_{D} of 500 nM or less. In still other embodiments, the antibody binds to CD44, or more preferably to the HA binding domain of CD44, with a K_{D} of 2×10⁻⁸ M, 2×10⁻⁹ M, or 5×10⁻¹⁰ M or less. In an even more preferred embodiment, the antibody binds to CD44, in the HA binding domain with a K_{D} of 2.5×10⁻¹² M or less. In some embodiments, the antibody binds to CD44 with substantially the same K_{D} as antibody 1A9.A6.B9; 2D1.A3.D12; 14G9.B8.B4; or 10C8.2.3.

In some embodiments, the anti-CD44 antibody has a low dissociation rate constant (k_{off}). In some embodiments, the anti-CD44 antibody binds to CD44, or more preferably to the HA binding domain of CD44, with a k_{off} of 1.0×10⁻³s⁻¹ or lower or a k_{off} of 5.0×10⁻⁴ s⁻¹ or lower. In still other embodiments, the k_{off} is substantially the same as an antibody described herein, including an antibody selected from 1A9.A6.B9; 2D1.A3.D12; 14G9.B8.B4; and 10C8.2.3. In some embodiments, the antibody binds to CD44, with substantially the same k_{off} as an antibody that comprises the CDR regions of a heavy chain, or the CDR regions of a light chain, from an antibody selected from 1A9.A6.B9; 2D1.A3.D12; 14G9.B8.B4; and 10C8.2.3. In some embodiments, the antibody binds to CD44, or more preferably to the HA binding domain of CD44, with substantially the same k_{off} as an antibody that comprises a heavy chain variable domain having the amino acid sequence of the V_{H} region found in SEQ ID NOs: 9, 45, 81 and 117 a light chain variable domain having the amino acid sequence of the V_{L} region found in SEQ ID NOs: 13, 49, 85 or 121. In still another embodiment, the antibody binds to CD44, or more preferably to the HA binding domain of CD44, with substantially the same k_{off} as an antibody that comprises the CDR regions of a light chain variable domain having the amino acid sequence of the V_{L} region found in SEQ ID NOs: 15, 49, 85 or 121; or the CDR regions of a heavy chain variable domain having the amino acid sequence of the V_{H} region found in SEQ ID NOs: 9, 45, 81 and 117.

The binding affinity and dissociation rate of an anti-CD44 antibody to CD44 can be determined by methods known in the art. The binding affinity can be measured by ELISAs, RIAs, flow cytometry (FACS), surface plasmon resonance, such as BIACORE™. The dissociation rate can be measured by surface plasmon resonance. Preferably, the binding affinity and dissociation rate are measured by surface plasmon resonance. More preferably, the binding affinity and dissociation rate are measured using BIACORE™. One can determine whether an antibody has substantially the same K_{D} as an anti-CD44 antibody by using methods known in the art. EXAMPLE 5 provides a method for determining affinity constants of anti-CD44 monoclonal antibodies.

### Identification of CD44 Epitopes Recognized by Anti-CD44 Antibodies

The invention provides a human anti-CD44 monoclonal antibody that binds to CD44 and competes or cross-competes with and/or binds the same epitope as: (a) an antibody selected from 1A9.A6.B9; 2D1.A3.D12; 14G9.B8.B4; and 10C8.2.3; (b) an antibody that comprises a heavy chain variable domain having an amino acid sequence of the variable domain found in SEQ ID NOs: 9, 45, 81 and 117; (c) an antibody that comprises a light chain variable domain having an amino acid sequence of the variable domain found in SEQ ID NOs: 13, 49, 85 or 121; or (d) an antibody that comprises both a heavy chain variable domain as defined in (b) and a light chain variable domain as defined in (c). If two antibodies reciprocally compete with each other for binding to CD44, they are said to cross-compete.

One can determine whether an antibody binds to the same epitope or cross-competes for binding with an anti-CD44 antibody by using methods known in the art. In one embodiment, one allows the anti-CD44 antibody of the invention to bind to CD44 under saturating conditions and then measures the ability of the test antibody to bind to CD44. If the test antibody is able to bind to CD44 at the same time as the anti-CD44 antibody, then the test antibody binds to a different epitope as the anti-CD44 antibody. However, if the test antibody is not able to bind to CD44 at the same time, then the test antibody binds to the same epitope, an overlapping epitope, or an epitope that is in close proximity to the epitope bound by the human anti-CD44 antibody. This experiment can be performed using an ELISA, a RIA, BIACORE™, or flow cytometry (FAGS).

To test whether an anti-CD44 antibody cross-competes with another anti-CD44 antibody, one may use the competition method described above in two directions, i.e., determining if the reference antibody blocks the test antibody and vice versa. In one embodiment, the experiment is performed using an ELISA. Methods of determining K_{D} are discussed further below.

### Inhibition of CD44 Activity by Anti-CD44 Antibodies

In another embodiment, the invention provides an anti-C D44 antibody that inhibits CD44-mediated signaling. In other embodiments, the invention provides an anti-CD44 antibody that inhibits the co-stimulatory signaling for lymphocytes and monocytes through CD44. In yet another embodiment the invention provides an anti-CD44 antibody that blocks cytokine production, and particularly cytokines such as TNF-α, IL-6 and IL-1β. In a further embodiment, the invention provides an anti-CD44 antibody that inhibits the binding of HA to the CD44 receptor. In one embodiment, the CD44 receptor is human. In still another embodiment, the anti-CD44 antibody is a human antibody. The IC₅₀ can be measured in a ligand binding assay by ELISA, RIA, or other assays and cell-based assays such as FACS assay or cells expressing CD44. In one embodiment, the antibody or antigen-binding portion thereof inhibits ligand binding between HA and CD44 with an IC₅₀ of no more than 5 μg/ml, preferably no more than 1 μg/ml; more preferably than 0.5 μg/ml, even more preferably no more than 0.20 μg/ml as measured by an ELISA assay. EXAMPLE 4 provides a method for determining inhibition by monoclonal antibodies of CD44 binding to HA.

In another embodiment, the invention provides an anti-CD44 antibody that prevents binding of CD44 to HA. In one embodiment, the anti-CD44 antibody inhibits HA-induced: (i) leukocyte recruitment; (ii) cell-matrix interactions and direct interactions between cells, such as for example, leukocytes and endothelial cells; (iii) regulation of leukocytes cell function; (iv) metabolism of HA; and/or (v) the contribution of CD44 to the assembly, organization and remodeling of matrix. One can determine whether an anti-CD44 antibody can prevent, inhibit or reduced activation of CD44 in the presence of HA by determining the inflammatory cytokine release from leukocytes triggered by lipopolysaccha ride (LPS) and HA. Assays for detecting CD44 activation and/or HA binding to CD44 are described in EXAMPLES 4, 5, 6 and 7. In one embodiment, one would determine the levels of CD44 activation using a cytokine assay. In some embodiments, the IC₅₀, measured using a HA competition binding assay, is no more than 5 μg/ml, preferably no more than 1 μg/ml, more preferably than 0.5 μg/ml, even more preferably no more than 0.20 μg/ml.

### Reduction of Surface Cell Expression by Anti-CD44 Antibodies

In another aspect of the invention, the antibody causes a downregulation of cell surface CD44 expression after incubation with the antibody. In an embodiment, the incubation can be a short time period (e.g., 4 hours) or a longer time period (e.g., 24 hours). Particularly, the present invention provides for an anti-CD44 antibody that induces downregulation of CD44 expression on circulating lymphocytes, and preferably, on CD3+ T lymphocytes. A downregulation of cell surface CD44 expression can be measured using FACS. In particular embodiments of the invention, the antibody may cause preferably a 6% decrease of cell surface CD44 expression, preferably a 10% decrease, or more preferably a 20% downregulation, or even more preferably at least 50% decrease of cell surface CD44 expression as measured by FACS. EXAMPLE 8 exemplifies one type of FACS assay measuring downregulation of cell surface CD44 expression on leukocyte and T-cells in two species: human and cynomolgus monkey.

### Methods of Producing Antibodies

Monoclonal antibodies of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (U.S. Pat. No. 4,816,567). To create a humanized antibody, the murine CDR regions can be inserted into a human framework using methods known in the art U.S. Pat. Nos. 5,225,539, 5,530,101; 5,585,089; 5,693,762; and 6,180,370.

In a preferred embodiment, the antibodies of the invention are human monoclonal antibodies. Such human monoclonal antibodies directed against CD44 can be generated using transgenic or transchromosomic mice carrying parts of the human immune system rather than the mouse system. These transgenic and transchromosomic mice include mice referred to herein as the HuMAb Mouse® and KM Mouse®, respectively, and are collectively referred to herein as “human Ig mice.”

The HuMAb Mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368: 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). Preparation and use of the HuMAb Mouse®, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851. See further, U.S. Pat. Nos. 5,545,806; 5,569,825; 5,625,126; 5,633,425; 5,789,650; 5,877,397; 5,661,016; 5,814,318; 5,874,299; and 5,770,429; U.S. Pat. No. 5,545,807; PCT Publication Nos.: WO 92/03918, WO 93/12227, WO 94/25585, WO 97/13852, WO 98/24884 and WO 99/45962; and WO 01/14424.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication No. WO 02/43478.

Still further, alternative transgenic animal systems expressing human immunoglobulin genes are available in the art and can be used to raise anti-CD44 antibodies of the invention. For example, an alternative transgenic system referred to as the Xenomouse™ (Abgenix, Inc.) can be used; such mice are described in, for example, U.S. Pat. Nos. 5,939,598; 6,075,181; 6,114,598; 6,150,584; and 6,162,963.

Moreover, alternative transchromosomic animal systems expressing human immunoglobulin genes are available in the art and can be used to raise anti-CD44 antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-CD44 antibodies of the invention.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996; and 5,698,767.

### Immunization of Human Ig Mice

### Production of Antibodies and Antibody-Producing Cell Lines

After immunization of an animal with a CD44 antigen, antibodies and/or antibody-producing cells can be obtained from the animal. In some embodiments, anti-CD44 antibody-containing serum is obtained from the animal by bleeding or sacrificing the animal. The serum may be used as it is obtained from the animal, an immunoglobulin fraction may be obtained from the serum, or the anti-CD44 antibodies may be purified from the serum.

In some embodiments, antibody-producing immortalized cell lines are prepared from cells isolated from the immunized animal. After immunization, the animal is sacrificed and lymph node and/or splenic B cells are immortalized by any means known in the art. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells are screened using CD44, a portion thereof, or a cell expressing CD44. In one preferred embodiment, the CD44 portion comprises: (i) the HA binding site of CD44; (ii) comprises the full or a truncated amino acid sequence set forth in SEQ ID NO:1 and/or SEQ ID NO:2; or (iii) combinations thereof. In one embodiment, the initial screening is performed using an enzyme-linked immunoassay (ELISA) or a radioimmunoassay. An example of ELISA, screening is provided in PCT Publication No. WO 00/37504.

Anti-CD44 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

In one embodiment, the immunized animal is a non-human animal that expresses human immunoglobulin genes and the splenic B cells are fused to a myeloma cell line from the same species as the non-human animal. In a more preferred embodiment, the immunized animal is a Kirin TC Mouse™ mouse and the myeloma cell line is a non-secretory mouse myeloma. In an even more preferred embodiment, the myeloma cell line is Sp2/0-Ag14 (American Type Culture Collection (ATCC)CRL-1581) and the mouse hybridoma cell line is 1376.3.2d1.A3.D12 (ATCC No. PTA-6928), 1376.3.1A9.A6.B9 (ATCC No. PTA-6929) or 1376.2.14G9.B8.B4 (ATCC No. PTA-6927). See, e.g., EXAMPLE 1.

Thus, in one embodiment, the invention provides methods for producing a cell line that produces a human monoclonal antibody or antigen binding portions thereof directed to CD44 comprising: (a) immunizing a non-human transgenic animal described herein with CD44, a portion of CD44 or a cell or tissue expressing CD44; (b) allowing the transgenic animal to mount an immune response to CD44; (c) isolating antibody-producing cells from the transgenic animal; (d) immortalizing the antibody-producing cells; (e) creating individual monoclonal populations of the immortalized antibody-producing cells; and (f) screening the immortalized antibody-producing cells to identify an antibody directed to CD44. In one embodiment, step (f) comprises screening the immortalized antibody-producing cells to identify an antibody directed to the HA binding site of CD44, and optionally, which does not bind outside the HA binding site of CD44.

Screening the immortalized antibody-producing cells to identify an antibody directed to the HA binding site of CD44 may be achieved by testing if the antibodies produced by the cell bind to a peptide comprising the amino acid sequence of the HA binding site of CD44.

In another aspect, the invention provides hybridomas that produce a human anti-CD44 antibody. In one embodiment, the human anti-CD44 antibody produced by the hybridoma is an antagonist of CD44. In still another embodiment, the human anti-CD44 antibody produced by the hybridoma (i) binds to the HA binding site of CD44; (ii) does not bind outside the HA binding site; (iii) does not bind to the IM7 binding site; or (iv) combinations thereof. Mikecz et al., (1999) Arthritis Rheumatism 42: 659, 668, Zheng (1995) J. Cell Biol. 130: 485-495, Peach et al., (1993) J. Cell Biol. 122: 257-264 and U.S. Pat. No. 6,001,356. In one embodiment, the hybridomas are mouse hybridomas, as described above. In other embodiments, the hybridomas are produced in other mammals.

In one embodiment of the invention, antibody-producing cells are isolated and expressed in a host cell, for example myeloma cells. In still another embodiment, a transgenic animal is immunized with CD44, primary cells, (e.g., spleen or peripheral blood cells) are isolated from an immunized transgenic animal and individual cells producing antibodies specific for the desired antigen are identified. Polyadenylated mRNA from each individual cell is isolated and reverse transcription polymerase chain reaction (RT-PCR) is performed using sense primers that anneal to variable region Sequences, e.g., degenerate primers that recognize most or all of the FR1 regions of human heavy and light chain variable region genes and anti-sense primers that anneal to constant or joining region sequences. cDNAs of the heavy and light chain variable domains are then cloned and expressed in any suitable host cell, e.g., a myeloma cell, as chimeric antibodies with respective immunoglobulin constant regions, such as the heavy chain and κ or λ constant domains. See Babcook, J. S. et al. (1996) Proc. Natl. Acad. Sci. USA 93: 7843-48. Anti CD44 antibodies may then be identified and isolated as described herein.

### Recombinant Methods of Producing Antibodies

An antibody, or antibody binding portion, of the invention can be prepared by recombinant expression of immunoglobulin light and heavy chain genes in a host cell. For example, to express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, to incorporate these genes into recombinant expression vectors and to introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397.

### Mutations and Modifications

To express the CD44 antibodies of the present invention, DNA fragments encoding V_{H} and V_{L} regions can first be obtained using any of the methods described above. Various, modifications, e.g. mutations, deletions, and/or additions can also be introduced into the DNA sequences using standard methods known to those of skill in the art. For example, mutagenesis can be carried out using standard methods, such as PCR-mediated mutagenesis, in which the mutated nucleotides are incorporated into the PCR primers such that the PCR product contains the desired mutations or site-directed mutagenesis.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant domain of an antibody. In some embodiments, the cysteine is canonical.

The antibodies may also be modified, e.g. in the variable domains of the heavy and/or light chains, e.g., to alter a binding property of the antibody. For example, a mutation may be made in one or more of the CDR regions to increase or decrease the K_{D} of the antibody for CD44, to increase or decrease k_{off}, or to alter the binding specificity of the antibody. Techniques in site-directed mutagenesis are well-known in the art. See, e.g., Sambrook et al. and Ausubel et al., supra.

A modification of mutation may also be made in a framework region or constant domain to increase the half-life of a CD44 antibody. See, e.g., PCT Publication No. WO 00/09560. A mutation in a framework region or constant domain can also be made to alter the immunogenicity of the antibody, to provide a site for covalent or non-covalent binding to another molecule, or to alter such properties as complement fixation, FcR binding and antibody-dependent cell-mediated cytotoxicity (ADCC). According to the invention, a single antibody may have mutations in any one or more of the CDRs or framework regions of the variable domain or in the constant domain.

In a process known as “germlining”, certain amino acids in the V_{H} and V_{L} sequences can be mutated to match those found naturally in germline V_{H} and V_{L} sequences. In particular, the amino acid sequences of the framework regions in the V_{H} and V_{L} sequences can be mutated to match the germline sequences to reduce the risk of immunogenicity when the antibody is administered. Germline DNA sequences for human V_{H} and V_{L} genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson et al. (1992) J. Mol. Biol. 227:776-798; and Cox et al., (1994) Eur. J. Immunol. 24:827-836.

Another type of amino acid substitution that may be made is to remove potential proteolytic sites in the antibody. Such sites may occur in a CDR or framework region of a variable domain or in the constant domain of an antibody. Substitution of cysteine residues and removal of proteolytic sites may decrease the risk of heterogeneity in the antibody product and thus increase its homogeneity. Another type of amino acid substitution is to eliminate asparagine-glycine pairs, which form potential deamidation sites, by altering one or both of the residues. In another example, the C-terminal lysine of the heavy chain of a CD44 antibody of the invention can be cleaved. In various embodiments of the invention, the heavy and light chains of the CD44 antibodies may optionally include a signal sequence.

Once DNA fragments encoding the V_{H} and V_{L} segments of the present invention are obtained, these DNA fragments can be further manipulated by standard recombinant DNA techniques, for example to convert the variable region genes to full-length antibody chain genes, to Fab fragment genes, or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG2 constant region. The IgG1 constant region sequence can be any of the various alleles or allotypes known to occur among different individuals, such as Gm(1), Gm(2), Gm(3), and Gm(17). These allotypes represent naturally occurring amino acid substitution in the IgG1 constant regions. For a Fab fragment heavy chain gene, the V_{H}-encoding DNA can be operatively linked to another DNA molecule encoding only the heavy chain CH1 constant region. The CH1 heavy chain constant region may be derived from any of the heavy chain genes.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region. The kappa constant region may be any of the various alleles known to occur among different individuals, such as Inv(1), Inv(2), and Inv(3). The lambda constant region may be derived from any of the three lambda genes.

To create a scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (See e.g., Bird et al., (1988) Science 242:423-426; Huston et al., (1988) Proc. Natl. Acad. Sci. USA 85:5879-5883; McCafferty et al., (1990) Nature 348:552-554. The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to CD44 and to another molecule.

In another embodiment, a fusion antibody or immunoadhesin may be made that comprises all or a portion of a CD44 antibody of the invention linked to another polypeptide. In another embodiment, only the variable domains of the CD44 antibody are linked to the polypeptide. In another embodiment, the V_{H} domain of a CD44 antibody is linked to a first polypeptide, while the V_{L} domain of a CD44 antibody is linked to a second polypeptide that associates with the first polypeptide in a manner such that the V_{H} and V_{L} domains can interact with one another to form an antigen binding site. In another preferred embodiment, the V_{H} domain is separated from the V_{L} domain by a linker such that the V_{H} and V_{L} domains can interact with one another. The V_{H}-linker-V_{L} antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

In other embodiments, other modified antibodies may be prepared using CD44 antibody encoding nucleic acid molecules. For instance, “Kappa bodies” (Ill et al., (1997) Protein Eng. 10: 949-57), “Minibodies” (Martin et al., (1994) EMBO J. 13: 5303-9), “Diabodies” (Holliger et al., (1993) Proc. Natl. Acad. Sci. USA 90: 6444-6448), or “Janusins” (Traunecker et al., (1991) EMBO J. 10:3655-3659 and Traunecker et al., (1992) Int. J. Cancer (Suppl.) 7:51-52) may be prepared using standard molecular biological techniques following the teachings of the specification.

Bispecific antibodies or antigen-binding fragments can be produced by a variety of methods including fusion of hybridomas or linking of Fab′ fragments. See, e.g., Songsivilai & Lachmann, (1990) Clin. Exp. Immunol. 79:315-321, Kostelny et al., (1992) J. Immunol. 148:1547-1553. In addition, bispecific antibodies may be formed as “diabodies” or “Janusins.” In some embodiments, the bispecific antibody binds to two different epitopes of CD44. In some embodiments, the modified antibodies described above are prepared using one or more of the variable domains or CDR regions from a human CD44 antibody provided herein.

### Vectors and Host Cells

To express the antibodies and antigen-binding portions of the invention, DNAs encoding partial or full-length light and heavy chains, obtained as described herein, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. Expression vectors include, for example, plasmids, retroviruses, adenoviruses, adeno-associated viruses (AAV), plant viruses such as cauliflower mosaic virus, tobacco mosaic virus, cosmids, YACs, EBV derived episomes. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human C_{H} or C_{L} immunoglobulin sequence, with appropriate restriction sites engineered so that any V_{H} or V_{L} sequence can easily be inserted and expressed, as described above. In such vectors, splicing usually occurs between the splice donor site in the inserted J region and the splice acceptor site preceding the human C domain, and also at the splice regions that occur within the human C_{H} exons. Polyadenylation and transcription termination occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, and so forth. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods for expressing antibodies in plants, including a description of promoters and vectors, as well as transformation of plants is known in the art. See, e.g., U.S. Pat. No. 6,517,529. Methods of expressing polypeptides in bacterial cells or fungal cells, e.g., yeast cells, are also well known in the art.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification), the neomycin phosphotransferase gene (for G418 selection), and the glutamate synthetase gene.

Nucleic acid molecules encoding CD44 antibodies and vectors comprising these nucleic acid molecules can be used for transfection of a suitable mammalian, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455. Methods of transforming plant cells are well known in the art, including, e.g., Agrobacterium-mediated transformation, biolistic transformation, direct injection, electroporation and viral transformation. Methods of transforming bacterial and yeast cells are also well known in the art.

Mammalian cell lines available as hosts for expression are well known in the art and include many immortalized cell lines available from the American Type Culture Collection (ATCC). These include, for example, Chinese hamster ovary (CHO) cells, NSO cells, SP2 cells, HEK-293T cells, NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 or Sf21 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Plant host cells include, e.g., Nicotiana, Arabidopsis, duckweed, corn, wheat, potato, and so forth. Bacterial host cells include E. coli and Streptomyces species. Yeast host cells include Schizosaccharomyces pombe, Saccharomyces cerevisiae and Pichia pastoris.

Further, expression of antibodies of the invention from production cell lines can be enhanced using a number of known techniques. For example, the glutamine synthetase (the GS system) and DHFR gene expression systems are common approaches for enhancing expression under certain conditions. High expressing cell clones can be identified using conventional techniques, such as limited dilution cloning and Microdrop technology. The GS system is discussed in European Patent Nos. EP 0 216 846, EP 0 256 055, EP 0 323 997 and EP 0 338 841.

It is likely that antibodies expressed by different cell lines or in transgenic animals will have different glycosylation from each other. However, all antibodies encoded by the nucleic acid molecules provided herein, or comprising the amino acid sequences provided herein are part of the present invention, regardless of the glycosylation of the antibodies.

### Phage Display Libraries

The invention provides a method for producing an anti-CD44 antibody or antigen-binding portion thereof comprising the steps of synthesizing a library of human antibodies on phage, screening the library with CD44 or a portion thereof, isolating phage that bind CD44, and obtaining the antibody from the phage. By way of example, one method for preparing the library of antibodies for use in phage display techniques comprises the steps of immunizing a non-human animal comprising human immunoglobulin loci with CD44 or an antigenic portion thereof to create an immune response, extracting antibody-producing cells from the immunized animal; isolating RNA encoding heavy and light chains of antibodies of the invention from the extracted cells, reverse transcribing the RNA to produce cDNA, amplifying the cDNA using primers, and inserting the cDNA into a phage display vector such that antibodies are expressed on the phage. Recombinant anti-CD44 antibodies of the invention may be obtained in this way.

Recombinant anti-CD44 human antibodies of the invention can be isolated by screening a recombinant combinatorial antibody library. Preferably the library is a scFv phage display library, generated using human V_{L} and V_{H} cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, and WO 92/09690; Fuchs et al., (1991) Bio/Technology 9:1370-1372; Hay et al., (1992) Hum. Antibod. Hybridomas 3:81-85; Huse et al., (1989) Science 246:1275-1281; McCafferty et al., (1990) Nature 348:552-554; Griffiths et al., (1993) EMBO J. 12:725-734; Hawkins et al., (1992) J. Mol. Biol. 226:889-896; Clackson et al., (1991) Nature 352:624-628; Gram et al., (1992) Proc. Natl. Acad. Sci. USA 89:3576-3580; Garrad et al., (1991) Bio/Technology 9:1373-1377; Hoogenboom et al.; (1991) Nuc. Acid Res. 19:4133-4137; and Barbas et al., (1991) Proc. Natl. Acad. Sci. USA 88:7978-7982.

In one embodiment to isolate and produce human anti-CD44 antibodies with the desired characteristics, a human anti-CD44 antibody as described herein is first used to select human heavy and light chain sequences having similar binding activity toward CD44, using the epitope imprinting methods described in PCT Publication No. WO 93/06213. The antibody libraries used in this method are preferably scFv libraries prepared and screened as described in PCT Publication No. WO 92/01047, McCafferty et al., Nature 348:552-554 (1990); and Griffiths et al., EMBO J. 12:725-734 (1993). The scFv antibody libraries preferably are screened using human CCR2 as the antigen.

Once initial human V_{L} and V_{H} domains are selected, “mix and match” experiments are performed, in which different pairs of the initially selected V_{I} and V_{H} segments are screened for CD44 binding to select preferred V_{L}N_{H} pair combinations. Additionally, to further improve the quality of the antibody, the V_{L} and V_{H} segments of the preferred V_{L}N_{H} pair(s) can be randomly mutated, preferably within the CDR3 region of V_{H} and/or V_{I}, in a process analogous to the in vivo somatic mutation process responsible for affinity maturation of antibodies during a natural immune response. This in vitro affinity maturation can be accomplished by amplifying V_{H} and V_{L} domains using PCR primers complimentary to the V_{H} CDR3 or V_{L} CDR3, respectively, which primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be re-screened for binding to CD44.

Following screening and isolation of an anti-CD44 antibody of the invention from a recombinant immunoglobulin display library, nucleic acids encoding the selected antibody can be recovered from the display package (e.g., from the phage genome) and subcloned into other expression vectors by standard recombinant DNA techniques. If desired, the nucleic acid can further be manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

### Deimmunized Antibodies

In another aspect of the invention, the antibodies or antigen binding portions thereof may be deimmunized to reduce their immunogenicity using the techniques described in, e.g., PCT Publication Nos.: WO98/52976 and WO00/34317.

### Derivatized and Labeled Antibodies

An anti-CD44 antibody or antigen-binding portion of the invention can be derivatized or linked to another molecule (e.g., another peptide or protein). In general, the antibodies or antigen-binding portion thereof are derivatized such that the CD44 binding is not affected adversely bythe derivatization or labeling. Accordingly, the antibodies and antigen-binding portions of the invention are intended to include both intact and modified forms of the human CD44 antibodies described herein. For example, an antibody or antigen-binding portion of the invention can be functionally linked (by chemical coupling, genetic fusion, noncovalent association or otherwise) to one or more other molecular entities, such as another antibody (e.g., a bispecific antibody or a diabody), a detection agent, a label, a cytotoxic agent, a pharmaceutical agent, a protein or peptide that can mediate association of the antibody or antigen-binding portion with another molecule (such as a streptavidin core region or a polyhistidine tag) and/or a carrier protein (e.g. blood protein, albumin or transferrin).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are commercially available from Pierce Chemical Company, Rockford, Ill.

Another type of derivatized antibody is a labeled antibody. Useful detection agents with which an antibody or antigen-binding portion of the invention may be derivatized include fluorescent compounds, including, for example, fluorescein, fluorescein isothiocyanate, rhodamine, 5-dimethylamine-1-naphthalenesulfonyl chloride, phycoerythrin, lanthanide phosphors. An antibody can also be labeled with enzymes that are useful for detection, such as, for example, horseradish peroxidase, β-galactosidase, luciferase, alkaline phosphatase, glucose oxidase. When an antibody is labeled with a detectable enzyme, it is detected by adding additional reagents that the enzyme uses to produce a reaction product that can be discerned. For example, when the agent horseradish peroxidase is present the addition of hydrogen peroxide and diaminobenzidine leads to a colored reaction product, which is detectable. An antibody can also be labeled with biotin, and detected through indirect measurement of avidin or streptavidin binding. An antibody can also be labeled with a predetermined polypeptide epitope recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. A CD44 antibody can also be derivatized with a chemical group such as polyethylene glycol (PEG), a methyl or ethyl group, or a carbohydrate group. These groups are useful to improve the biological characteristics of the antibody, e.g., to increase serum half-life.

### Pharmaceutical Compositions and Administration

This invention also provides a pharmaceutical composition for the treatment of abnormal cell infiltration in a mammal, including a human, comprising an amount of a CD44 antibody or antigen binding portion thereof, as described herein, that is effective in treating abnormal cell infiltration, and a pharmaceutically acceptable carrier. The preferred compositions provide a therapeutic benefit to patients with one of more of a variety of inflammatory and autoimmune diseases, such as rheumatoid arthritis, Juvenile Rheumatoid Arthritis, atherosclerosis, granulmatous diseases, multiples sclerosis, asthma, Crohn's Disease, Ankylosing Spondylitis, Psoriatic Arthritis, Plaque Psoriasis and cancer.

The antibodies and antigen-binding portions of the present invention can be incorporated into pharmaceutical compositions suitable for administration to a subject as described in, e.g. PCT publication WO 2006/096488 and references cited therein. Typically, the pharmaceutical composition comprises an antibody or antigen-binding portion of the invention and a pharmaceutically acceptable carrier adapted to maintain protein stability, solubility and bioactivity. As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives, buffers including amino acids, and chelating agents, e.g. EDTA, DTPA, DFM and mixtures thereof, which enhance the shelf life or effectiveness of the antibody. In one embodiment, a pharmaceutical composition includes an IgG, preferably an IgG₁ or IgG₂, monoclonal antibody and a pharmaceutically acceptable chelating agent. A representative molar concentration of the antibody ranges from about 0.0006 millimolar to about 1.35 millimolar and the moler concentration of the chelating agent ranges from about 0.003 millimolar to about 50 millimolar and the moler ratio of antibody to chelating agent ranges from about 0.00001 to about 2000.

The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection. Formulations for injection may be presented in unit dosage form, e.g., in ampoules or in multi-dose containers, with or without an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the CD44 antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies or antigen-binding portions of the present invention can be administered by a variety of methods known in the art, although for many therapeutic applications, the preferred route/mode of administration is subcutaneous, intramuscular, or intravenous infusion. As will be appreciated by the skilled artisan, the route and/or mode of administration will vary depending upon the desired results.

In certain embodiments, the antibody compositions of the present invention may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. See, e.g., Sustained and Controlled Release Drug Delivery Systems J. R. Robinson, ed., Marcel Dekker, Inc., New York, 1978.

Additional active compounds also can be incorporated into the compositions. In certain embodiments, an inhibitory CD44 antibody of the invention is co-formulated with and/or co-administered with one or more additional therapeutic agents. These agents include, without limitation, antibodies that bind other targets, anti-tumor agents, anti-angiogenesis agents, signal transduction inhibitors, anti-proliferative agents, chemotherapeutic agents, or peptide analogues that inhibit CD44. Such combination therapies may require lower dosages of the inhibitory CD44 antibody as well as the co-administered agents, thus avoiding possible toxicities or complications associated with the various monotherapies.

The present compounds may also be used in co-therapies (pretreatment, post-treatment or concurrent treatment), partially or completely, in addition to other anti-inflammatories or DMARDS, including put not limited to cyclosporine, zoledronic acid, efalizumab, alefacept, etodolac, lornoxicam, OM-89, valdecoxib, tocilizumab, abatacept, meloxicam, etanercept, nambumetone, rimexolone, 153Sm-EDTMP, prosorba, imidazole salicylate, oprelvekin, hylauronic acid, naproxen, piroxicam, diacerein, lumericoxib, rofecoxib tacrolimus, aceclofenac, actarit, tenoxicam, rosiglitazone, deflazacort, adalimumab, leflunomide, risedronate sodium, misoprostol and diclofenac, SK-1306X, infliximab, anakinra, celecoxib, diclofenac, etoricoxib and felbinac, reumacon, golimumab, denosumab, ofatumumab, 10rT1 antibody, pelubiprofen, licofelone, temsirolimus, eculizumab, iguratimod, methylprednisolone acetate, ibuprofen, triamcinolone acetonide, nabumetone, oxaprozin, oxycodone hcl, fentanyl, sulindac, pyridoxine, acetaminophen, alendronate, indomethacin, glucosamine, olopatadine, omeprazol, Azathioprine, Sulfasalazine, Hydroxychloroquine, Ciclosporin and prednisone. Other suitable anti-inflammatories include those designated by company code number such as 480156S, AA861, AD1590, AFP802, AFP860, AI77B, AP504, AU8001, BPPC, BW540C, CHINOIN 127, CN100, EB382, EL508, F1044, FK-506, GV3658, ITF182, KCNTEI6090, KME4, LA2851, MR714, MR897, MY309, ON03144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770, CP-481715, ABN-912, MLN-3897, HuMax-IL-15, RA-1, paclitaxel, Org-37663, Org 39141, AED-9056, AMG-108, fontolizumab, pegsunercept, pralnacasan, apilimod, GW-274150, AT-001, 681323 (GSK) K-832, R-1503, ocrelizumab, DE-096, Cpn10, THC+CBD (GW Pharma), 856553 (GSK), ReN-1869, immunoglobulin, mm-093, amelubant, SCIO-469, ABT-874, LenkoVAX, LY-2127399, TRU-015, KC-706, amoxapinet and dipyridamole, TAK-715, PG 760564, VX-702, prednisolone and dipyridamole, PMX-53, belimumab, prinaberel, CF-101, tgAAV-TNFR:Fc, R-788, prednisolone and SSRI, CP-690550 and PMI-001.

In another embodiment, additional therapeutic agents include biological agents. In a further embodiment, one or more biological agents are selected from a tumor necrosis factor-alpha (TNF-α) antagonist, an interleukin-1alpha (IL-1α) antagonist, a CD28 antagonist and a CD20 antagonist. In yet a further embodiment, one or more biological agents are selected from the group consisting of etanercept (ENBREL™), adalimumab (HUMIRAT™), infliximab (REMICADE™), anakinra (KINERET™), abatacept (ORENCIA™), rituximab (RITUXAN™) and certolizumab pegol (CIMZIAT™).

Examples of other pharmaceutically active agents which may be employed in combination with compounds of formula (I) and their salts and solvates for rheumatoid arthritis therapy include: immunosuppresants such as amtolmetin guacil, mizoribine and rimexolone; anti-TNF.alpha. agents such as etanercept, infliximab, diacerein; tyrosine kinase inhibitors such as leflunomide; kallikrein antagonists such as subreum; interleukin 11 agonists such as oprelvekin; interferon beta 1 agonists; hyaluronic acid agonists such as NRD-101 (Aventis); interleukin 1 receptor antagonists such as anakinra; CD8 antagonists such as amiprilose hydrochloride; beta amyloid precursor protein antagonists such as reumacon; matrix metalloprotease inhibitors such as cipemastat and other disease modifying anti-rheumatic drugs (DMARDs) such as methotrexate, sulphasalazine, cyclosporin A, hydroxychoroquine, auranofin, aurothioglucose, gold sodium thiomalate and penicillamine.

The compositions of the invention may include a “therapeutically effective amount” or a “prophylactically effective amount” of an antibody or antigen-binding portion of the invention. A “therapeutically effective amount” refers to an amount effective, at dosages and for periods of time necessary, to achieve the desired therapeutic result. A therapeutically effective amount of the antibody or antigen-binding portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antigen-binding portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the CD44 antibody or antigen-binding portion thereof and the particular therapeutic or prophylactic effect to be achieved, and (b) the limitations inherent in the art of compounding such an antibody for the treatment of sensitivity in individuals.

An exemplary, non-limiting range for a therapeutically or prophylactically effective amount of an antibody or antibody portion of the invention is 0.025 to 50 mg/kg, more preferably 0.1 to 50 mg/kg, more preferably 0.1-25, 0.1 to 10 or 0.1 to 3 mg/kg. In one embodiment, the antibody or antibody portion of the invention is administered in a formulation as a sterile aqueous solution having a pH that ranges from about 5.0 to about 6.5 and comprising from about 1 mg/ml to about 200 mg/ml of antibody, from about 1 millimolar to about 100 millimolar of, for example, histidine, acetate, or succinate buffer, from about 0.01 mg/ml to about 10 mg/ml of polysorbate 80, from about 100 millimolar to about 400 millimolar of trehalose, and from about 0.01 millimolar to about 1.0 millimolar of disodium EDTA dihydrate. The compositions of the present invention optionally may comprise a pharmaceutically acceptable antioxidant and/or a chelating agent. Suitable antioxidants include, but are not limited to, methionine, sodium thiosulfate, catalase, and platinum. For example, the composition may contain methionine in a concentration that ranges from 1 mM to about 100 mM, and in particular, is about 27 mM. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another aspect of the present invention provides kits comprising a CD44 antibody or antigen-binding portion of the invention or a composition comprising such an antibody or antigen-binding portion. A kit may include, in addition to the antibody or composition, diagnostic or therapeutic agents. A kit can also include instructions for use in a diagnostic or therapeutic method. In a preferred embodiment, the kit includes the antibody or a composition comprising it and a diagnostic agent that can be used in a method described below. In another preferred embodiment, the kit includes the antibody or a composition comprising it and one or more therapeutic agents that can be used in a method described below.

### Diagnostic Methods of Use

In another aspect, the invention provides in vivo and in vitro diagnostic methods. The anti-CD44 antibodies can be used to detect CD44 in a biological sample in vitro or in vivo. In one embodiment, the invention provides a method for diagnosing the presence or location of a CD44-expressing cells in a subject in need thereof, comprising the steps of injecting the antibody into the subject, determining the expression of CD44 in the subject by localizing where the antibody has bound, comparing the expression in the subject with that of a normal reference subject or standard, and diagnosing the presence or location of the cells. The anti-CD44 antibodies may also be used as a marker for inflammation and/or for the infiltration of immune cells, such as monocytes and T cells, into a tissue.

The anti-CD44 antibodies can be used in a conventional immunoassay, including, without limitation, an ELISA, a RIA, flow cytometry, tissue immunohistochemistry, a Western blot or an immunoprecipitation. The anti-CD44 antibodies of the invention can be used to detect CD44 from humans. In another embodiment, the anti-CD44 antibodies can be used to detect CD44 from cynomolgus monkeys or rhesus monkeys.

The invention provides a method for detecting CD44 in a biological sample comprising contacting the biological sample with an anti-CD44 antibody of the invention and detecting the bound antibody. In one embodiment, the anti-CD44 antibody is directly labeled with a detectable label. In another embodiment, the anti-CD44 antibody (the first antibody) is unlabeled and a second antibody or other molecule that can bind the anti-CD44 antibody is labeled. As is well known to one of skill in the art, a second antibody is chosen that is able to specifically bind the particular species and class of the first antibody. For example, if the anti-CD44 antibody is a human IgG, then the secondary antibody could be an anti-human-IgG. Other molecules that can bind to antibodies include, without limitation, Protein A and Protein G, both of which are available commercially, e.g., from Pierce Chemical Company.

In other embodiment, CD44 can be assayed in a biological sample by a competition immunoassay utilizing CD44 standards labeled with a detectable substance and an unlabeled anti-CD44 antibody. In this assay, the biological sample, the labeled CD44 standards and the anti-CD44 antibody are combined and the amount of labeled CD44 standard bound to the unlabeled antibody is determined. The amount of CD44 in the biological sample is inversely proportional to the amount of labeled CD44 standard bound to the anti-CD44 antibody.

One can use the immunoassays disclosed in the application for a number of purposes. For example, the anti-CD44 antibodies can be used to detect CD44 in cultured cells. In one embodiment, the anti-CD44 antibodies are used to determine the amount of CD44 on the surface of cells that have been treated with various compounds. This method can be used to identify compounds that modulate CD44 protein levels. According to this method, one sample of cells is treated with a test compound for a period of time while anothersample is left untreated. If the total CD44 expression is to be measured, the cells are lysed and the total CD44 expression is measured using one of the immunoassays described above. The total CD44 expression in the treated versus the untreated cells is compared to determine the effect of the test compound.

A preferred immunoassay for measuring total CD44 expression is flow cytometry or immunohistochemistry. If the cell surface CD44 expression is to be measured, the cells are not lysed, and the cell surface levels of CD44 are measured using one of the immunoassays described above. A preferred immunoassay for determining cell surface levels of CD44 includes the steps of labeling the cell surface proteins with a detectable label, such as biotin or ¹²⁵I, immunoprecipitating the CD44 with an anti-CD44 antibody and then detecting the labeled CD44.

Another preferred immunoassay for determining the localization of CD44, e.g., cell surface levels, is by using immunohistochemistry. A preferred immunoassay to detect cell surface levels of CD44 includes binding of an anti-CD44 antibody labeled with an appropriate fluorophore, such as fluorescein or phycoerythrin, and detecting the primary antibody using flow cytometry. In another embodiment, the anti-CD44 antibody is unlabeled and a second antibody or other molecule that can bind the anti-CD44 antibody is labeled Methods such as ELISA, RIA, flow cytometry, Western blot, immunohistochemistry, cell surface labeling of integral membrane proteins and immunoprecipitation are well known in the art (see, e.g., Harlow and Lane, supra). In addition, the immunoassays can be scaled up for high throughput screening in order to test a large number of compounds for either activation or inhibition of CD44.

The anti-CD44 antibodies of the invention also can be used to determine the levels of CD44 in a tissue or in cells derived from the tissue. In some embodiments, the tissue is a diseased tissue. In some embodiments, the tissue is a tissue biopsy. In some embodiments of the method, a tissue or a biopsy thereof is excised from a patient. The tissue or biopsy is then used in an immunoassay to determine, e.g., total CD44 expression, cell surface levels of CD44 or localization of CD44 by the methods discussed above. Such methods can be used to determine whether a tissue expresses high levels of CD44, which could be indicative that the tissue is a target for treatment with anti-CD44 antibody.

The antibodies of the present invention also can be used in vivo to identify tissues and organs that express CD44. In some embodiments, the anti-CD44 antibodies are used to identify CD44-expressing cells. One advantage of using the human anti-CD44 antibodies of the present invention is that they may safely be used in vivo without eliciting a substantial immune response to the antibody upon administration, unlike antibodies of non-human origin or with humanized or chimeric antibodies.

The method comprises the steps of administering a detectably labeled anti-CD44 antibody or a composition comprising them to a patient in need of such a diagnostic test and subjecting the patient to imaging analysis to determine the location of the CD44-expressing tissues. Imaging analysis is well known in the medical art, and includes, without limitation, x-ray analysis, magnetic resonance imaging (MRI) or computed tomography (CT). The antibody can be labeled with any agent suitable for in vivo imaging, for example a contrast agent, such as barium, which can be used for x-ray analysis, or a magnetic contrast agent, such as a gadolinium chelate, which can be used for MRI or CT. Other labeling agents include, without limitation, radioisotopes, such as ⁹⁹Tc. In another embodiment, the anti-CD44 antibody will be unlabeled and will be imaged by administering a second antibody or other molecule that is detectable and that can bind the anti-CD44 antibody. In embodiment, a biopsy is obtained from the patient to determine whether the tissue of interest expresses CD44.

In an embodiment, the detectably labeled anti-CD44 comprises a fluorophore.

In yet a further embodiment, the anti-CD44 antibodies of the present invention may also be used to determine the reduction in surface cell expression of CD44 on cells. In a preferred embodiment, the cells are lymphocytes and monocytes.

### Therapeutic Methods of Use

In another embodiment, the invention provides a method for inhibiting CD44 activity by administering a CD44 antibody to a patient in need thereof. Any of the antibodies or antigen-binding portions thereof described herein may be used therapeutically. In an embodiment, the CD44 antibody is a chimeric or humanized antibody. In a preferred embodiment, the CD44 is human and the patient is a human patient. Alternatively, the patient may be a mammal, e.g. a monkey, that expresses a CD44 that the CD44 antibody cross-reacts with. The antibody may be administered to a non-human mammal expressing CD44 as an animal model of human disease. Such animal models may be useful for evaluating the therapeutic efficacy of antibodies of this invention.

In another embodiment, a CD44 antibody or antibody portion thereof may be administered to a patient who expresses inappropriately high levels of CD44. The antibody may be administered once, but more preferably is administered multiple times for optimal efficacy. The antibody may be administered from three times daily to once every six months or longer. The administering may be on a schedule such as three times daily, twice daily, once daily, once every two days, once every three days, once weekly, once every two weeks, once every month, once every two months, once every three months and once every six months. The antibody may also be administered continuously via a minipump. The antibody may be administered via a mucosal, buccal, intranasal, inhalable, intravenous, subcutaneous, intramuscular, parenteral, or intratumor route. The antibody may be administered once, at least twice or for at least the period of time until the condition is treated, palliated or cured. The antibody generally will be administered for as long as the condition is present. The antibody will generally be administered as part of a pharmaceutical composition as described supra. The dosage of antibody will generally be in the range of 0.1 to 100 mg/kg, more preferably 0.5 to 50 mg/kg, more preferably 1 to 20 mg/kg, and even more preferably 1 to 10 mg/kg. The serum concentration of the antibody may be measured by any method known in the art.

This invention also provides a method for the treatment of abnormal cell infiltration in a mammal, including a human, comprising administering to said mammal a therapeutically effective amount of a CD44 antibody or antigen binding portion thereof, as described herein, that is effective in treating abnormal cell infiltration.

### Gene Therapy

The nucleic acid molecules that encode the antibodies and antibody portions of the present invention can be administered to a patient in need thereof via gene therapy. The therapy may be either in vivo or ex vivo. In a preferred embodiment, nucleic acid molecules encoding both a heavy chain and a light chain are administered to a patient. In a more preferred embodiment, the nucleic acid molecules are administered such that they are stably integrated into chromosomes of B cells because these cells are specialized for producing antibodies. In a preferred embodiment, precursor B cells are transfected or infected ex vivo and re-transplanted into a patient in need thereof. In another embodiment, precursor B cells or other cells are infected in vivo using a virus known to infect the cell type of interest. Typical vectors used for gene therapy include liposomes, plasmids, and viral vectors. Exemplary viral vectors are retroviruses, adenoviruses and adeno-associated viruses. After infection either in vivo or ex vivo, levels of antibody expression can be monitored by taking a sample from the treated patient and using any immunoassay known in the art or discussed herein.

In a preferred embodiment, the gene therapy method comprises the steps of administering an isolated nucleic acid molecule encoding the heavy chain or an antigen-binding portion thereof of a CD44 antibody and expressing the nucleic acid molecule. In another embodiment, the gene therapy method comprises the steps of administering an isolated nucleic acid molecule encoding the light chain or an antigen-binding portion thereof of a CD44 antibody and expressing the nucleic acid molecule. In a more preferred method, the gene therapy method comprises the steps of administering an isolated nucleic acid molecule encoding the heavy chain or an antigen-binding portion thereof and an isolated nucleic acid molecule encoding the light chain or the antigen-binding portion thereof of a CD44 antibody of the invention and expressing the nucleic acid molecules. The gene therapy method may also comprise the step of administering another therapeutic agent, such as any of the agents discussed in the present application in connection with combination therapy.

In order that this invention may be even better understood, the following examples are set forth. These examples are for purposes of illustration only and are not to be construed as limiting the scope of the invention in any manner.

### EXAMPLES

In the following examples and preparations, “BSA” means bovine serum albumin; “EDTA” means ethylenediaminetetraacetic acid; “DMSO” means dimethyl sulfoxide; “MOPS” means 3-(N-morpholino) propanesulfonic acid; “MES” means 2-(N-Morpholino)ethanesulfonic acid; “PBS” means phosphate buffered saline; “dPBS” means Dulbecco's phosphate buffered saline; “HEMA” means 2-hydroxy-ethyl methacrylate; “DMEM” means Dulbecco's modified eagle's medium; “FBS” means fetal bovine serum; “NEAA” means non-essential amino acids; “HEPES” means N-2-hydroxyethylpiperazine-N′-2-ethanesulfonic acid; and “DMF” means dimethyl formamide.

### Example 1

### Generation of Hybridomas Producing Anti-CD44 Antibody

Preferred antibodies in accordance with the invention were prepared, selected, and assayed as follows:

### Immunization and Hybridoma Generation:

Purified recombinant human CD44-Ig fusion protein (SEQ ID NO:1), murine pre-B cell line, 300-19 (Reth, M. G. et al, Nature 312 29: 418-42, 1984; Alt, F. et al., Cell 27: 381-390, 1981), transfected to express human CD44 and the human monocytic leukemia cell line, THP-1 (ATCC Cat. No. TIB-202), which naturally express human CD44, were used as immunogens.

Fully human monoclonal antibodies to human CD44 were prepared using human Ig transgenic mouse strains HCo7 and HCo12, as well as the human transchromosomal/transgenic strain, KM (Mederex, Inc.). These strains all express fully human antibodies that are indistinguishable from antibodies isolated from humans. In these mouse strains, the endogenous mouse kappa light chain gene has been homozygously disrupted as described in Chen et al. (1993) EMBO J. 12:811-820 and the endogenous mouse heavy chain gene has been homozygously disrupted as described in EXAMPLE 1 of PCT Publication WO 01/09187. Each of these mouse strains carries a human kappa light chain transgene, KCo5, as described in Fishwild et al. (1996) Nature Biotechnology 14:845-851. The HCo7 strain carries the HCo7 human heavy chain transgene as described in U.S. Pat. Nos. 5,545,806; 5,625,825; and 5,545,807. The HCo12 strain carries the HCo12 human heavy chain transgene as described in EXAMPLE 2 of PCT Publication WO 01/09187. The KM strain carries a human mini-chromosome as described in lshida et al., (2002), Cloning and Stem Cells, 4: 91-102.

To generate fully human monoclonal antibodies to CD44, HuMab mice of the HCo7, HCo12 and KM strain, were immunized with THP-1 cells, purified recombinant CD44-Fc or 300-19 transfectants expressing human CD44. General immunization schemes for HuMab mice are described in Lonberg, N. et al (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. A purified recombinant preparation of CD44-Fc antigen (5-20 μg), a preparation of THP-1 cells or transfected 300-19 cells (1×10⁷ cells) was used to immunize the HuMab mice intraperitonealy (IP), subcutaneously (Sc) or via footpad injection (fp).

Transgenic mice were immunized with antigen in Ribi adjuvant intraperitonealy and subcutaneously in 1-4 weeks intervals (up to a total of 8 immunizations). The immune response was monitored in blood taken by retro orbital bleeds. The serum was screened by FACS (as described below), and mice with sufficient titers of anti-CD44 human immunoglobulin were used for fusions. Mice were boosted intravenously with antigen 3 and 2 days before sacrifice and removal of the spleen and/or lymph nodes. Typically, 10-20 fusions for each antigen were performed. A total of 81 HCo7, HCo12 and KM mice were immunized. Several dozen mice were immunized for each antigen.

### Selection of HuMab Mice Producing Anti-CD44 Antibodies:

To select HuMab mice producing antibodies that bound CD44, sera from immunized mice were screened by flow cytometry (FACS) for binding to a cell line expressing full length human CD44, and not to a control cell line not expressing CD44: Briefly, CD44-expressing 300-19 cells were incubated with serum from immunized mice diluted at 1:20. Cells were washed and specific antibody binding was detected with FITC-labeled anti-human IgG Ab. Flow cytometric analyses were performed on a FACS flow cytometry instrument (Becton Dickinson, San Jose, Calif.). Mice that developed the highest titers of anti-CD44 antibodies were used for fusions. Fusions were performed as described below and hybridoma supernatants were tested for anti-CD44 activity by FACS.

### Generation of Hybridomas Producing Human Monoclonal Antibodies to CD44:

The mouse splenocytes and/or lymph node lymphocytes, isolated from the HuMab mice, were fused using polyethylene glycol (PEG) or electrofusion (E-fusion, Cyto Pulse™ technology, Cyto Pulse™ Sciences, Inc., Glen Burnie, Md.) to the mouse myeloma cell line, SP2/0 (ATCC, CRL-1581, Vendor, City, State), using standard or manufacturer recommended protocols. Briefly, single cell suspensions of splenic and/or lymph node lymphocytes from immunized mice were fused to between one-third and equal number of Sp2/0 nonsecreting mouse myeloma cells using 50% PEG (Sigma, St. Louis, Mo.) or E-fusion, respectively. Cells were plated at approximately 1×10⁵ splenocytes/well (PEG) or 2×10⁴ splenocytes/well (E-Fusion) in flat bottom microtiter plate, and incubated for 10-14 days in selective medium containing 10% fetal bovine serum, 10% P388D1 (ATCC, CRL-TIB-63) conditioned medium, 3-5% (IGEN) in DMEM (Mediatech, Herndon, Va., Cat.No. CRL 10013, with high glucose, L-glutamine and sodium pyruvate), 5 mM HEPES, 0.055 mM 2-mercaptoethanol, 50 mg/ml gentamycin and 1×HAT (Sigma, Cat. No. CRL-P-7185). After 1-2 weeks, cells were cultured in medium in which the HAT was replaced with HT. Approximately 10-14 days after cell plating supernatants from individual wells were screened first for whether they contained human gamma, kappa antibodies. The supernatants which were scored positive for human gamma, kappa were then subsequently screened by FACS (described above) for human anti-CD44 monoclonal IgG antibodies. The antibody secreting hybridomas were transferred to 24 well plates, screened again and, if confirmed positive for human anti-CD44 IgG monoclonal antibodies, were subcloned at least twice by limiting dilution. The stable subclones were then cultured in vitro to generate small amounts of antibody in tissue culture medium for further characterization.

The hybridomas were deposited on Aug. 10, 2005, in accordance with the Budapest Treaty with the American Type Culture Collection (ATCC), 10801 University Blvd., Manassas, Va. 20110-2209, and in accordance with the conditions of deposit under 37 C.F.R. §§1.801-1.809. In addition, vectors containing cDNA corresponding to mAb 10C8.2.3 were deposited with the ATCC on Jun. 15, 2006, in accordance with the same conditions, under ATTC designations PTA-7658 and 7659. All restrictions upon public access to the deposits will be irrevocably removed upon grant of a patent on this application and the deposits will be replaced if viable samples cannot be dispensed by the depository.

The hybridomas have been assigned the following accession numbers:

TABLE 3

| | Mouse Hybridoma | | |
|---|---|---|---|
| | Cell Line | | Strain |
| Antibody | Designation | ATCC Designation | Designation |
| 1A9.A6.B9 | 1376.3.1A9.A6.B9 | ATCC No. PTA-6927 | LN 15922 |
| 2D1.A3.D12 | 1376.3.2d1.A3.D12 | ATCC No. PTA-6929 | LN 15920 |
| 14G9.B8.B4 | 1376.2.14G9.B8.B4 | ATCC No. PTA-6928 | LN 15921 |

### Example 2

Cloning of Human and Cynomolgus CD44 cDNA to Generate Stable Cell Lines and CD44-Ig Fusion Proteins
Human CD44 cDNA Cloning:

Human CD44 (SEQ ID NO:1) was cloned from human spleen cDNA (Clontech Labs. Inc., Mountain View, Calif., Cat. No. 639312) with the following primers: 5′-atggacaagttttggtggcacgcagcctgg-3′ (SEQ ID NO:155) and 5′-ttacaccccaatcttcatgtccaca-3′ (SEQ ID NO:156). The PCR product was re-amplified using following primers to add XhoI and XbaI restriction sites: 5′-gactcgaggccaccatggacaagttttggtggc-3′ (SEQ ID:157) and 5′-gatctagatcactattacaccccaatcttcatgtcc-3′ (SEQ ID NO:158). This second PCR product was ligated into a pMIG mammalian expression vector and CD44 sequence was verified in both stands. Hawley et al., (1994) Gene Thera. 1:136-138.

Cynomolgus CD44 cDNA Cloning:

Cynomolgus CD44 gene was PCR amplified from cyno PBMC cDNA with the following primers: 5′-atggacaagttttggtgg-3′ (SEQ ID NO:159) and 5′-gttacaccccaatcttcatgtcca-3′ (SEQ ID NO:160). PCR product was ligated into PCR2.1 TOPO vector (Invitrogen, City, Carlsbad, Calif., Cat. No. K4510-20). Nineteen clones were sequenced and cynoCD44 sequence was determined by consensus sequence of all above clones. The nucleic acid sequence of two clones, 5-2 (SEQ ID NO:153) and 5-8 (SEQ ID NO:8) are shown. Sequence verified cynomolgus CD44 (SEQ ID NO:8) was subcloned into a mammalian expression vector pMIG. Hawley et al., (1994).

### 300-19 Human and Cynomolgus CD44 300-19 Overexpression Cell Line:

Both pMIG-human CD44 and pMIG-cynoCD44 were transfected into 293T/17 cells (ATCC No. CRL-11263) with FUGENE 6 transfection reagent (Hoffman-La Roche Inc., Nutley, N.J., Cat. No. 11815091001) generated human CD44 and cynoCD44 retroviruses. Both retroviruses were subsequently transduced into 300-19 cells to generate human CD44 and cyno CD44 expression cell lines.

### Cloning of Human CD44-IgG1 Fusion Protein:

The extracellular domain of human CD44 was expressed as a human IgG1 fusion protein (SEQ ID NO:3). The cDNA encoding the mature extracellular domain of CD44 was PCR amplified (Klentaq PCR kit, Clontech Labs Inc., Mountain View, Calif., Cat. No. 639108) from human leukocyte cDNA (Clontech Labs. Inc.) and subcloned into a mammalian expression vector—PCDMamp containing a CD5 leader sequence and a human IgG1 tag. The following PCR primers were designed to published sequence of the CD44 standard form (G. R. Screaton, et al., (1992) PNAS 89:12160-12164,)

(SEQ ID NO: 161)

(CD44 + C: AGTGAGACTAGTCAGATCGATTTGAATATAACCTGCCGC
TTTG),

(SEQ ID NO: 162)

(CD44 − D:
ATCACTGAGATCTTCTGGAATTTGGGGTGTCCTTATAG).

The complete CD44IgG1 cDNA was sequenced and verified in both strands.

### Cynomolgus CD44-IgG1 Protein Cloning:

The extracellular domain of cynomolgus CD44 was expressed as a human IgG1 Fc fusion protein (SEQ ID NO:5). The cDNA encoding the mature extracellular domain of cyno CD44 was PCR amplified from pMIG-cynoCD44 vector and subcloned into an in-house mammalian expression vector pLNp that contains a CD5 leader sequence, an human IgG1 tag as well as XhoI and Hpal restriction sites. PCR Primers were designed to align with human CD44 extracellular domain with XhoI and EcoRV restriction sites. The primers sequences are as follow: 5′-atcggcgatccagatcgatttgaatataacc-3′ (SEQ ID NO:163), 5′-ctgtgcctcgagccattctggaatttggggtgtcc-3′ (SEQ ID NO:164). The complete cyno CD44 extacelluar IgG1 cDNA was sequence verified in both strands.

PCR condition for all above cloning used platinum Tag polymerase (Invitrogen, Cat. No. 11304-011) followed by standard PCR protocol: 3 minutes at 95° C.; 25× (30 seconds at 55° C., 1 minute at 78° C.); 7 minutes at 72° C.

Extracellular Domain of humanCD44 and cynoCD44 Expression and Purification:

Both human CD44IgG1 (SEQ ID NO:3) and cynoCD44 IgG1 fusion protein (SEQ ID NO: 5) were expressed using the Freestyle 293 Expression System (Invitrogen, Cat. No K9000-01) according to the manufacturer's protocols.

The fusion protein was purified on Protein A agarose beads. (Pierce, Rockford, Ill., Cat. No. 15918-014). After the culture media was harvested, protease inhibitor tablets (Hoffman La-Roche Cat. No. 1 697 498, 1 tab/50 ml media), Tris buffer (pH 8.0, final concentration, 10 mM) and sodium azide (final concentration, 0.02%) were added and filtered through a 0.22 micron filter. One ml of 50% slurry of Protein A beads was added to every 100 ml media. Rotate media/slurry mixture for at least 2 hours at 4° C. Spinning at 1000×g for 10 minutes resulted in pellet agarose. The supernatant was carefully remove and the agarose pellet was resuspended in 2-3 volumes of wash buffer (0.1M Tris HCL pH7.5, 0.1M NaCl) and applied to column. The column was washed with 20 bed volumes of wash buffer and eluted with 5 bed volumes of elution buffer (ImmunoPure IgG Elution Buffer, Pierce, Cat. No. 21004) into a tube containing ½ column volume of 1M Tris pH8. Buffer was exchanged into PBS, using Amicon concentrators 10,000 MW cutoff (Millipore, Billerica, Mass.) according to manufacturer's protocol.

### Example 3

Sequences of Anti-CD44 Antibodies Prepared in Accordance with the Invention

To analyze the structure of antibodies produced in accordance with the invention, we cloned nucleic acids encoding heavy and light chain fragments from hybridomas producing anti-CD44 monoclonal antibodies. Cloning and sequencing was accomplished as follows:

Poly(A)+ mRNA was silated using an RNeasy Mini Kit (Qiagen, San Diego, Calif.) and cDNA synthesized from the mRNA with the Advantage RT-for-PCR kit (BD Biosciences, Franklin Lakes, N.J.) using oligo(dT) priming. The oligo(dT) primed cDNA for clone 1A9.A6.B9, 2D1.A3.D12, and 14G9.B8.B4 were amplified using degenerate primers listed in TABLES 4, 5, and 6, respectively. Amplification was achieved using the High Fidelity Polymerase (Roche) and a PTC-200 DNA Engine (MJ Research) with cycling as follows: 2′@95° C.; 25× (20″@95° C., 30″@52° C., 2′@72° C.); 10′@72° C. PCR amplicons were cloned into the pCR2.1 TOPO (Invitrogen, Carlsbad, Calif. Cat. No. K4500-01) and transformed into TOP10 chemically competent cells (Invitrogen) using the standard protocol. Clones were sequence verified using Grills 16^{th} BDTv3.1/dGTP chemistry (Applied Biosystems Inc) and a 3730×1 DNA Analyzer (Applied Biosystems Inc., Foster, Calif.). All sequences were analysed by alignments to the ‘V BASE sequence directory’ (Tomlinson, et al, (1992) J. Mol. Biol., 227, 776-798; Hum, (1995) Mol. Genet., 3, 853-860; EMBO J., 14, 4628-4638.

TABLE 4
Degenerate primers (5′ to 3′) for 1A9.A6.B9

| | |
|---|---|
| VH3c_5UTR_F | ATTYRGTGATCAGSACTGAACASAG (SEQ ID |
| | NO: 165) |
| G_3UTR_R | TACGTGCCAAGCATCCTCGC (SEQ ID NO: 166) |
| VK3_5UTR_F | ATCAATGCCTGKGTCAGAGCYYTG (SEQ ID |
| | NO: 167) |
| K_3UTR_R | AGGCTGGAACTGAGGAGCAGGTG (SEQ ID |
| | NO: 168) |

TABLE 5
Degenerate primers (5′ to 3′) for 2D1.A3.D12

| | |
|---|---|
| VH1a_5UTR_F | CCCTGAGAGCATCAYMYARMAACC (SEQ ID |
| | NO: 169) |
| G_3UTR_R | TACGTGCCAAGCATCCTCGC (SEQ ID NO: 170) |
| VK1a_5UTR_F | GSARTCAGWCYCWVYCAGGACACAGC (SEQ ID |
| | NO: 171) |
| K_3UTR_R | AGGCTGGAACTGAGGAGCAGGTG (SEQ ID |
| | NO: 172) |

TABLE 6
Degenerate primers (5′ to 3′) for 14G9.B8.B4

| | |
|---|---|
| VH1a_5UTR_F | CCCTGAGAGCATCAYMYARMAACC (SEQ ID |
| | NO: 173) |
| G_3UTR_R | TACGTGCCAAGCATCCTCGC (SEQ ID NO: 174) |
| VK3_5UTR_F | ATCAATGCCTGKGTCAGAGCYYTG (SEQ ID |
| | NO: 175) |
| K_3UTR_R | AGGCTGGAACTGAGGAGCAGGTG (SEQ ID |
| | NO: 176) |

### Gene Utilization:

TABLE 7 sets forth the gene utilization evidenced by selected hybridoma clones of antibodies in accordance with the invention.

TABLE 7

| | Heavy chain | | | Light chain | | |
|---|---|---|---|---|---|---|
| Clone | V_{H} | D | J_{H} | V_{L} | J_{K} | Isotype |
| 1A9.A6.B9 | 3-33 | D4-17 | JH6b | L6 | JK4 | IgG2 |
| 2D1.A3.D12 | 1-03 | nd | JH6b | L19 | JK1 | IgG1 |
| 14G9.B8.B4 | 1-03 | D3-10 | JH5b | A27 | JK4 | IgG1 |
| 10C8.2.3 | 3-21 | D6-19 | JH6b | A27 | JK4 | IgG4 |

nd = not determined

### Sequence and Mutation Analysis:

As will be appreciated by those skilled in the art, gene utilization analysis provides only a limited overview of antibody structure. As the B-cells in the KM animals stochastically generate V-D-J heavy and V-J kappa light chain transcripts, there are a number of secondary processes that occur, including, without limitation, somatic hypermutation, deletions, N-additions, and CD3 extensions. See, for example, Mendez et al., (1997) Nature Genetics 15:146-156 and PCT Publication WO 98/24893. Accordingly, to further examine antibody structure, we generated predicted amino acid sequences of the antibodies from the cDNAs obtained from the clones.

FIG. 2 shows the alignment of predicted amino acid sequences of the heavy and light chain variable domains of isolated anti CD44 monoclonal antibodies with germline amino acid sequences of the corresponding light and heavy chain genes.

TABLES 9-12 provide the nucleotide and predicted amino acid sequences of the heavy and kappa light chains of antibodies 1A9.A6.B9 (TABLE 9), 2D1.A3.D12 (TABLE 10), 14G9.B8.B4 (TABLE 11), and 10C8.2.3 (TABLE 12) with the variable regions for each of the antibodies shown in uppercase.

We generated one mutated antibody 2D1.A3.D12. The heavy chain in antibody 2D1.A3.D12 was mutated to change a threonine residue at position 28 to an isoleucine. The light chain of antibody 2D1.A3.D12 at position 38 was mutated changing a glutamine residue to a histidine.

Mutagenesis, in the V_{H} (I28T) and V_{K} (H38Q) regions of clone 2D1.A3.D12, was conducted with the primers listed in TABLE 8 using the QuickChange Site Directed Mutagenesis Kit from Stratagen, according to the manufacturer's instructiuons. Mutations were confirmed by automated sequencing, and mutagenized inserts were subcloned into expression vectors. Mutagenesis of Anti-CD44 Antibody 2D1.A3.D12 was conducted as follows:

TABLE 8
Mutagenic primers (5′ to 3′) for 2D1.A3.D12

| | |
|---|---|
| 2D1_VH_I28T | AAGGCTTCTGGATACAcCTTCACTAGCTATGCT |
| | (SEQ ID NO: 177) |
| 2D1_VH_I28T_R | AGCATAGCTAGTGAAGgTGTATCCAGAAGCCTT |
| | (SEQ ID NO: 178) |
| 2D1_VL_H38Q | TTAGCCTGGTATCAGCAgAAACCAGGGAAAGCC |
| | (SEQ ID NO: 179) |
| 2D1_VL_H38Q_R | GGCTTTCCCTGGTTTcTGCTGATACCAGGCTAA |
| | (SEQ ID NO: 180) |

TABLE 9
DNA and protein sequences of antibody 1A9.A6.B9

| | |
|---|---|
| DESCRIPTION: | SEQUENCE: |

| | |
|---|---|
| DNA sequence of | CAGGTGCAGCTGGTGGAGTCTGGGGGAGGCGTGGTCCAGCCTGGGAGG |
| heavy chain from | TCCCTGAGACTCTCCTGTGCAGCGTCTGGATTCACCTTCAGTAGCTAT |
| hybridoma cells | GGCATGCACTGGGTCCGCCAGGCTCCAGGCAAGGGGCTGGAGTGGGTG |
| (variable domain | GCAGTTATATGGTATGATGGAAGTAATAAATTCTATGCAGACTCCGTG |
| in uppercase) | AAGGGCCGATTCACCATCTCCAGAGACAATTCCAAGAACACGCTGTAT |
| | CTGCAAATGAACAGCCTGAGAGCCGAGGACACGGCTGTGTATTACTGT |
| | GCGAGGAGAAGTGACTACAGGGGCTACTACGGTATGGACGTCTGGGGC |
| | CAAGGGACCACGGTCACCGTCTCCTCAgcctccaccaagggcccatcg |
| | gtcttccccctggcgccctgctccaggagcacctccgagagcacagcg |
| | gccctgggctgcctggtcaaggactacttccccgaaccggtgacggtg |
| | tcgtggaactcaggcgctctgaccagcggcgtgcacaccttcccagct |
| | gtcctacagtcctcaggactctactccctcagcagcgtggtgaccgtg |
| | ccctccagcaacttcggcacccagacctacacctgcaacgtagatcac |
| | aagcccagcaacaccaaggtggacaagacagttgagcgcaaatgttgt |
| | gtcgagtgcccaccgtgcccagcaccacctgtggcaggaccgtcagtc |
| | ttcctcttccccccaaaacccaaggacaccctcatgatctcccggacc |
| | cctgaggtcacgtgcgtggtggtggacgtgagccacgaagaccccgag |
| | gtccagttcaactggtacgtggacggcgtggaggtgcataatgccaag |
| | acaaagccacgggaggagcagttcaacagcacgttccgtgtggtcagc |
| | gtcctcaccgttgtgcaccaggactggctgaacggcaaggagtacaag |
| | tgcaaggtctccaacaaaggcctcccagcccccatcgagaaaaccatc |
| | tccaaaaccaaagggcagccccgagaaccacaggtgtacaccctgccc |
| | ccatcccgggaggagatgaccaagaaccaggtcagcctgacctgcctg |
| | gtcaaaggcctctaccccagcgacatcgccgtggagtgggagagcaat |
| | gggcagccggagaacaactacaagaccacacctcccatgctggactcc |
| | gacggctccttcttcctctacagcaagctcaccgtggacaagagcagg |
| | tggcagcaggggaacgtcttctcatgctccgtgatgcatgaggctctg |
| | cacaaccactacacgcagaagagcctctccctgtctccgggtaaa |
| | (SEQ ID NO: 10) |
| Derived protein | QVQLVESGGGVVQPGRSLRLSCAASGFTFSSYGMHWVRQAPGKGLEWV |
| sequence (by | AVIWYDGSNKFYADSVKGRFTISRDNSKNTLYLQMNSLRAEDTAVYYC |
| translation) of the | ARRSDYRGYYGMDVWGQGTTVTVSSastkgpsvfplapcsrstsesta |
| heavy chain from | algclvkdyfpepvtvswnsgaltsgvhtfpavlqssglyslssvvtv |
| hybridoma cells | pssnfgtqtytcnvdhkpsntkvdktverkccvecppcpappvagpsv |
| (variable domain | flfppkpkdtlmisrtpevtcvvvdvshedpevqfnwyvdgvevhnak |
| in uppercase) | tkpreeqfnstfxvvsvltvvhqdwlngkeykckvsnkglpapiekti |
| | sktkgqprepqvytlppsreemtknqvsltclvkgfypsdiavewesn |
| | gqpennykttppmldsdgsfflyskltvdksrwqqgnvfscsvmheal |
| | hnhytqkslslspgk (SEQ ID NO: 9) |
| DNA sequence of | Full Length Light Chain Sequence of 1A9.A6.B9 - Nucleotide |
| light chain from | Sequence |
| hybridoma cells | GAAATTGTGTTGACACAGTCTCCAGCCACCCTGTCTTTGTCTCCAGGG |
| (variable domain | GAAAGAGCCACCCTCTCCTGCAGGGCCAGTCAGAGTGTTATCAACTAC |
| in uppercase) | TTAGCCTGGTACCAACAGAAACCTGGCCAGGCTCCCAGGCTCCTCATC |
| | TATGATGCATCCAACAGGGCCTCTGGCATCCCAGCCAGGTTCAGTGGC |
| | AGTGGGTCTGGGACAGACTTCACTCTCACCATCAGCAGCCTAGAGCCT |
| | GAAGATTTTGCAGTTTATTACTGTCAGCAGCGTCGCAACTGGCCGCTC |
| | ACTTTCGGCGGAGGGACCAAGGTGGAGATCAAACgaactgtggctgca |
| | ccatctgtcttcatcttcccgccatctgatgagcagttgaaatctgga |
| | actgcctctgttgtgtgcctgctgaataacttctatcccagagaggcc |
| | aaagtacagtggaaggtggataacgccctccaatcgggtaactcccag |
| | gagagtgtcacagagcaggacagcaaggacagcacctacagcctcagc |
| | agcaccctgacgctgagcaaagcagactacgagaaacacaaagtctac |
| | gcctgcgaagtcacccatcagggcctgagctcgcccgtcacaaagagc |
| | ttcaacaggggagagtgt |
| | (SEQ ID NO: 14) |
| Derived protein | EIVLTQSPATLSLSPGERATLSCRASQSVINYLAWYQQKPGQAPRLLI |
| sequence (by | YDASNRASGIPARFSGSGSGTDFTLTISSLEPEDFAVYYCQQRRNWPL |
| translation) of | TFGGGTKVEIKrtvaapsvfifppsdeqlksgtasvvcllnnfyprea |
| light chain from | kvqwkvdnalqsgnsqesvteqdskdstyslsstltlskadyekhkvy |
| hybridoma cells | acevthqglsspvtksfnrgec |
| (variable domain | (SEQ ID NO: 13) |
| in uppercase) | |

TABLE 10
DNA and protein sequences of antibody 2D1.A3.D12

| | |
|---|---|
| DESCRIPTION: | SEQUENCE: |

| | |
|---|---|
| DNA sequence of | CAGGTCCAACTTGTGCAGTCTGGGGCTGAGGTGAAGAAGCCTGGGGCC |
| heavy chain from | TCAGTGAAGGTTTCCTGCAAGGCTTCTGGATACATCTTCACTAGCTAT |
| hybridoma cells | GCTATGCATTGGGTGCGCCAGGCCCCCGGACAAAGGCTTGAGTGGATG |
| (variable domain | GGGTGGATCAACGCTGCCATTGGTAGCACAAAATATTCACAGAAGTTC |
| in uppecase) | CAGGGCAGAGTCACCATTACCAGGGACACATCCGCGAGCACAGCCTAC |
| | ATGGAGCTGAGCAGCCTGAGATCTGAAGACACGGCTGTGTATTACTGT |
| | GCGAGAGACGGGTGGGAGGACTACTACTACCACGGTATGGACGTCTGG |
| | GGCCAAGGGACCACGGTCACCGTCTCCTCAgcctccaccaagggccca |
| | tcggtcttccccctggcaccctcctccaagagcacctctgggggcaca |
| | gcggccctgggctgcctggtcaaggactacttccccgaaccggtgacg |
| | gtgtcgtggaactcaggcgccctgaccagcggcgtgcacaccttcccg |
| | gctgtcctacagtcctcaggactctactccctcagcagcgtggtgacc |
| | gtgccctccagcagcttgggcacccagacctacatctgcaacgtgaat |
| | cacaagcccagcaacaccaaggtggacaagaaagttgagcccaaatct |
| | tgtgacaaaactcacacatgcccaccgtgcccagcacctgaactcctg |
| | gggggaccgtcagtcttcctcttccccccaaaacccaaggacaccctc |
| | atgatctcccggacccctgaggtcacatgcgtggtggtggacgtgagc |
| | cacgaagaccctgaggtcaagttcaactggtacgtggacggcgtggag |
| | gtgcataatgccaagacaaagccgcgggaggagcagtacaacagcacg |
| | taccgtgtggtcagcgtcctcaccgtcctgcaccaggactggctgaat |
| | ggcaaggagtacaagtgcaaggtctccaacaaagccctcccagccccc |
| | atcgagaaaaccatctccaaagccaaagggcagccccgagaaccacag |
| | gtgtacaccctgcccccatcccgggatgagctgaccaagaaccaggtc |
| | agcctgacctgcctggtcaaaggcttctatcccagcgacatcgccgtg |
| | gagtgggagagcaatgggcagccggagaacaactacaagaccacgcct |
| | cccgtgctggactccgacggctccttcttcctctacagcaagctcacc |
| | gtggacaagagcaggtggcagcaggggaacgtcttctcatgctccgtg |
| | atgcatgaggctctgcacaaccactacacgcagaagagcctctcccctg |
| | tctccgggtaaa (SEQ ID NO: 46) |
| Derived protein | QVQLVQSGAEVKKPGASVKVSCKASGYIFTSYAMHWVRQAPGQRLEWM |
| sequence (by | GWINAAIGSTKYSQKFQGRVTITRDTSASTAYMELSSLRSEDTAVYYC |
| translation) of | ARDGWEDYYYHGMDVWGQGTTVTVSSastkgpsvfplapsskstsggt |
| heavy chain from | aalgclvkdyfpepvtvswnsgaltsgvhtfpavlqssglyslssvvt |
| hybridoma cells | vpssslgtqtyicnvnhkpsntkvdkkvepkscdkthtcppcpapell |
| (variable domain | ggpsvflfppkpkdtlmisrtpevtcvvvdvshedpevkfnwyvdgve |
| in uppercase) | vhnaktkpreeqynstyrvvsvltvlhqdwlngkeykckvsnkalpap |
| | iektiskakgqprepqvytlppsrdeltknqvsltclvkgfypsdiav |
| | ewesngqpennykttppvldsdgsfflyskltvdksrwqqgnvfscsv |
| | mhealhnhytqkslslspgk (SEQ ID NO: 45) |
| DNA sequence of | GACATCCAGATGACCCAGTCTCCATCTTCCGTGTCTGCATCTGTAGGA |
| light chain from | GACAGAGTCACCATCACTTGTCGGGCGAGTCAGGGTATTAGTAGCTGG |
| hybridoma cells | TTAGCCTGGTATCAGCATAAACCAGGGAAAGCCCCTAAGCTCCTGATC |
| (variable domain | TATGCTGCATCCAGTTTGCAAAGTGGGGTCCCATCAAGGTTCAGCGGC |
| in uppercase) | AGTGGATCTGGGACAGATTTCACTCTCACCATCAGCAGCCTGCAGCCT |
| | GAAGATTTTGCAACTTACTATTGTCAACAGGCTAATAATTTCCCGTGG |
| | ACGTTCGGCCAAGGGACCAAGGTGGAAATCAAACgaactgtggctgca |
| | ccatctgtcttcatcttcccgccatctgatgagcagttgaaatctgga |
| | actgcctctgttgtgtgcctgctgaataacttctatcccagagaggcc |
| | aaagtacagtggaaggtggataacgccctccaatcgggtaactcccag |
| | gagagtgtcacagagcaggacagcaaggacagcacctacagcctcagc |
| | agcaccctgacgctgagcaaagcagactacgagaaacacaaagtctac |
| | gcctgcgaagtcacccatcagggcctgagctcgcccgtcacaaagagc |
| | ttcaacaggggagagtgt (SEQ ID NO: 50) |
| Derived protein | DIQMTQSPSSVSASVGDRVTITCRASQGISSWLAWYQHKPGKAPKLLI |
| sequence (by | YAASSLQSGVPSRFSGSGSGTDFTLTISSLQPEDFATYYCQQANNFPW |
| translation) of | TFGQGTKVEIKrtvaapsvfifppsdeqlksgtasvvcllnnfyprea |
| light chain from | kvqwkvdnalqsgnsqesvteqdskdstyslsstltlskadyekhkvy |
| hybridoma cells | acevthqglsspvtksfnrgec (SEQ ID NO: 49) |
| (variable domain | |
| in uppercase) | |

TABLE 11
DNA and protein sequences of antibody 14G9.B8.B4

| | |
|---|---|
| DESCRIPTION: | SEQUENCE: |

| | |
|---|---|
| DNA sequence of | CAGGTCCAGCTTGTGCAGTCTGGGGCTGAGGTGAAGAAGCCTGGGGCC |
| heavy chain from | TCAGTGAAGGTTTCCTGCAAGGCTTCTGGATACACCTTCACTAACTAT |
| hybridoma cells | GCTATGCATTGGGTGCGCCAGGCCCCCGGACAAAGGCTTGAGTGGATG |
| (variable domain | GGATGGATCAACACTGGCAATGGTAACACAAAATATTCACAGAAGTTC |
| in uppercase) | CAGGGCAGAGTCACCATTACCAGGGACACATCCGCGAGCACAGCCTAC |
| | ATGGAGCTGAGCAGCCTGAGATCTGAAGACACGGCTGTGTATTACTGT |
| | GCGAGGTTTTACTCTGGTTCGGGGAGTCCCTGGGGCCAGGGAACCCTG |
| | GTCACCGTCTCCTCAgcctccaccaagggcccatcggtcttccccctg |
| | gcaccctcctccaagagcacctctgggggcacagcggccctgggctgc |
| | ctggtcaaggactacttccccgaaccggtgacggtgtcgtggaactca |
| | ggcgccctgaccagcggcgtgcacaccttcccggctgtcctacagtcc |
| | tcaggactctactccctcagcagcgtggtgaccgtgccctccagcagc |
| | ttgggcacccagacctacatctgcaacgtgaatcacaagcccagcaac |
| | accaaggtggacaagaaagttgagcccaaatcttgtgacaaaactcac |
| | acatgcccaccgtgcccagcacctgaactcctggggggaccgtcagtc |
| | ttcctcttccccccaaaacccaaggacaccctcatgatctcccggacc |
| | cctgaggtcacatgcgtggtggtggacgtgagccacgaagaccctgag |
| | gtcaagttcaactggtacgtggacggcgtggaggtgcataatgccaag |
| | acaaagccgcgggaggagcagtacaacagcacgtaccgtgtggtcagc |
| | gtcctcaccgtcctgcaccaggactggctgaatggcaaggagtacaag |
| | tgcaaggtctccaacaaagccctcccagcccccatcgagaaaaccatc |
| | tccaaagccaaagggcagccccgagaaccacaggtgtacaccctgccc |
| | ccatcccgggatgagctgaccaagaaccaggtcagcctgacctgcctg |
| | gtcaaaggcttctatcccagcgacatcgccgtggagtgggagagcaat |
| | gggcagccggagaacaactacaagaccacgcctcccgtgctggactcc |
| | gacggctccttcttcctctacagcaagctcaccgtggacaagagcagg |
| | tggcagcaggggaacgtcttctcatgctccgtgatgcatgaggctctg |
| | cacaaccactacacgcagaagagcctctccctgtctccgggtaaa |
| | (SEQ ID NO: 82) |
| Derived protein | QVQLVQSGAEVKKPGASVKVSCKASGYTFTNYAMHWVRQAPGQRLEWM |
| sequence (by | GWINTGNGNTKYSQKFQGRVTITRDTSASTAYMELSSLRSEDTAVYYC |
| translation) of | ARFYSGSGSPWGQGTLVTVSSastkgpsvfplapsskstsggtaalgc |
| heavy chain from | lvkdyfpepvtvswnsgaltsgvhtfpavlqssglyslssvvtvpsss |
| hybridoma cells | lgtqtyicnvnhkpsntkvdkkvepkscdkthtcppcpapellggpsv |
| (variable domain | flfppkpkdtlmisrtpevtcvvvdvshedpevkfnwyvdgvevhnak |
| in uppercase) | tkpreeqynstyrvvsvltvlhqdwlngkeykckvsnkalpapiekti |
| | skakgqprepqvytlppsrdeltknqvsltclvkgfypsdiavewesn |
| | gqpennykttppvldsdgsfflyskltvdksrwqqgnvfscsvmheal |
| | hnhytqkslslspgk (SEQ ID NO: 81) |
| DNA sequence of | GAAATTGTGTTGACGCAGTCTCCAGGCACCCTGTCTTTGTCTCCAGGG |
| 14G9.B8.B4 light | GAAAGAGCCACCCTCTCCTGCAGGGCCAGTCAGAGTGTTAGCAGCAGC |
| chain from | TACTTAGCCTGGTACCAGCAGAAACCTGGCCAGGCTCCCAGGCTCCTC |
| hybridoma cells | ATCTATGGTGCATCCAGCAGGGCCACTGGCATCCCAGACAGGTTCAGT |
| (variable domain | GGCAGTGGGTCTGGGACAGACTTCACTCTCACCATCAGCAGACTGGAG |
| in uppercase) | CCTGAAGATTTTGCAGTGTATTACTGTCAGCAGTATGGTAGCTCACCG |
| | CTCACTTTCGGCGGAGGGACCAAGGTGGAGATCAAACgaactgtggct |
| | gcaccatctgtcttcatcttcccgccatctgatgagcagttgaaatct |
| | ggaactgcctctgttgtgtgcctgctgaataacttctatcccagagag |
| | gccaaagtacagtggaaggtggataacgccctccaatcgggtaactcc |
| | caggagagtgtcacagagcaggacagcaaggacagcacctacagcctc |
| | agcagcaccctgacgctgagcaaagcagactacgagaaacacaaagtc |
| | tacgcctgcgaagtcacccatcagggcctgagctcgcccgtcacaaag |
| | agcttcaacaggggagagtgt (SEQ ID NO: 86) |
| Derived protein | EIVLTQSPGTLSLSPGERATLSCRASQSVSSSYLAWYQQKPGQAPRLL |
| sequence (by | IYGASSRATGIPDRFSGSGSGTDFTLTISRLEPEDFAVYYCQQYGSSP |
| translation) of | LTFGGGTKVEIKrtvaapsvfifppsdeqlksgtasvvcllnnfypre |
| light chain from | akvqwkvdnalqsgnsqesvteqdskdstyslsstltlskadyekhkv |
| hybridoma cells | yacevthqglsspvtksfnrgec |
| (variable domain | (SEQ ID NO: 85) |
| in uppercase) | |

TABLE 12
DNA and protein sequences of antibody 10C8.2.3

| | |
|---|---|
| DESCRIPTION: | SEQUENCE: |

| | |
|---|---|
| DNA sequence of | GAGGTGCAGCTGATGGAGTCTGGGGGAGGCCTGGTCAAGCCTGGGGGG |
| heavy chain from | TCCCTGAGACTCTCCTGTGCAGCCTCTGGATTCACCTTCAGTAGCTAT |
| hybridoma cells | AGCATGAACTGGGTCCGCCAGGCTCCAGGGAAGGGGCTGGAGTGGGTC |
| (variable domain | TCATCCATTACTGTTAGAAGTAGTTACATATACTACGCAGACTCAGTG |
| in uppercase) | AAGGGCCGATTCACCATCTCCAGAGACAACGCCAAGAACTCACTGTAT |
| | CTGCAAATGAACAGCCTGAGAGCCGAGGACACGGCTGTGTATTACTGT |
| | GCGAGAGTCCTCGCTATAGCAGTGCCTGGTACCTCCTACTACTACTAC |
| | GGTATGGACGTCTGGGGCCAAGGGACCACGGTCACCGTCTCCTCAgct |
| | tccaccaagggcccatccgtcttccccctggcgccctgctccaggagc |
| | acctccgagagcacagccgccctgggctgcctggtcaaggactacttc |
| | cccgaaccggtgacggtgtcgtggaactcaggcgccctgaccagcggc |
| | gtgcacaccttcccggctgtcctacagtcctcaggactctactccctc |
| | agcagcgtggtgaccgtgccctccagcagcttgggcacgaagacctac |
| | acctgcaacgtagatcacaagcccagcaacaccaaggtggacaagaga |
| | gttgagtccaaatatggtcccccatgcccatcatgcccagcacctgag |
| | ttcctggggggaccatcagtcttcctgttccccccaaaacccaaggac |
| | actctcatgatctcccggacccctgaggtcacgtacgtggtggtggac |
| | gtgagccaggaagaccccgaggtccagttcaactggtacgtggatggc |
| | gtggaggtgcataatgccaagacaaagccgcgggaggagcagttcaac |
| | agcacgtaccgtgtggtcagcgtcctcaccgtcctgcaccaggactgg |
| | ctgaacggcaaggagtacaagtgcaaggtctccaacaaaggcctcccg |
| | tcctccatcgagaaaaccatctccaaagccaaagggcagccccgagag |
| | ccacaggtgtacaccctgcccccatcccaggaggagatgaccaagaac |
| | caggtcagcctgacctgcctggtcaaaggcttctaccccagcgacatc |
| | gccgtggagtgggagagcaatgggcagccggagaacaactacaagacc |
| | acgcctcccgtgctggactccgacggctccttcttcctctacagcagg |
| | ctaaccgtggacaagagcaggtggcaggaggggaatgtcttctcatgc |
| | tccgtgatgcatgaggctctgcacaaccactacacacagaagagcctc |
| | tccctgtctctgggtaaa |
| | (SEQ ID NO: 118) |
| Derived protein | EVQLMESGGGLVKPGGSLRLSCAASGFTFSSYSMNWVRQAPGKGLEWV |
| sequence (by | SSITVRSSYIYYADSVKGRFTISRDNAKNSLYLQMNSLRAEDTAVYYC |
| translation) of | ARVLAIAVPGTSYYYYGMDVWGQGTTVTVSSastkgpsvfplapcsrs |
| heavy chain from | tsestaalgclvkdyfpepvtvswnsgaltsgvhtfpavlqssglysl |
| hybridoma cells | ssvvtvpssslgtktytcnvdhkpsntkvdkrveskygppcpscpape |
| (variable domain | flggpsvflfppkpkdtlmisrtpevtcvvvdvsqedpevqfnwyvdg |
| in uppercase) | vevhnaktkpreeqfnstyrvvsvltvlhqdwlngkeykckvsnkglp |
| | ssiektiskakgqprepqvytlppsqeemtknqvsltclvkgfypsdi |
| | avewesngqpennykttppvldsdgsfflysrltvdksrwqegnvfsc |
| | svmhealhnhytqkslslslgk (SEQ ID NO: 117) |
| DNA sequence of | GAAATTGTGTTGACGCAGTCTCCAGGCACCCTGTCTTTGTCTCCAGGG |
| light chain from | GAAAGAGCCACCCTCTCCTGCAGGGCCAGTCAGAGTGTTAGCAGCAGC |
| hybridoma cells | TACTTAGCCTGGTACCAGCAGAAACCTGGCCAGGCTCCCAGGCTCCTC |
| (variable domain | ATCTATGGTGCATCCAGCAGGGCCACTGGCATCCCAGACAGGTTCAGT |
| in uppercase) | GGCAGTGGGTCTGGGACAGACTTCACTCTCACCATCAGCAGACTGGAG |
| | CCTGAAGATTTTGCAGTGTATTACTGTCAGCAGTATGGTAGCTCACGG |
| | CTCACTTTCGGCGGAGGGACCAAGGTGGAGATCAAAcgaactgtggct |
| | gcaccatctgtcttcatcttcccgccatctgatgagcagttgaaatct |
| | ggaactgcctctgttgtgtgcctgctgaataacttctatcccagagag |
| | gccaaagtacagtggaaggtggataacgccctccaatcgggtaactcc |
| | caggagagtgtcacagagcaggacagcaaggacagcacctacagcctc |
| | agcagcaccctgacgctgagcaaagcagactacgagaaacacaaagtc |
| | tacgcctgcgaagtcacccatcagggcctgagctcgcccgtcacaaag |
| | agcttcaacaggggagagtgt (SEQ ID NO: 122) |
| Derived protein | EIVLTQSPGTLSLSPGERATLSCRASQSVSSSYLAWYQQKPGQAPRLL |
| sequence (by | IYGASSRATGIPDRFSGSGSGTDFTLTISRLEPEDFAVYYCQQYGSSR |
| translation) of | LTFGGGTKVEIKrtvaapsvfifppsdeqlksgtasvvcllnnfypre |
| light chain from | akvqwkvdnalqsgnsqesvteqdskdstyslsstltlskadyekhkv |
| hybridoma cells | yacevthqglsspvtksfnrgec (SEQ ID NO: 121) |
| (variable domain | |
| in uppercase) | |

Variable Domains of Anti-CD44 Antibodies were Cloned into Expression Vectors as Follows:

The variable domains were amplified from pCR2.1 cloned cDNA using primers listed in TABLE 13, 14, and 15. Amplification was achieved using the Pfx Platinum polymerase (Invitrogen) and a PTC-200 DNA Engine (MJ Research) with cycling as follows: 2 minutes at 94° C.; 20× (30 seconds at 94° C., 45 seconds at 55° C., 1 minute at 68° C.); minutes at 68° C. The variable domains were then cloned into expression vectors containing constant domains of the appropriate isotype. These clones were sequence verified using Grills 16^{th} BDTv3.1/dGTP chemistry (Applied Biosystems Inc) and a 3730×1 DNA Analyzer (Applied Biosystems Inc).

TABLE 13
Variable domain primers (5′ to 3′) for 1A9.A6.B9

| | |
|---|---|
| H3_11 | CAGGTGCAGCTGGTGGAGTCTGG (SEQ ID |
| | NO: 181) |
| G½_VH_R | TGGAGGCTGAGGAGACGGTGAC (SEQ ID NO: 182) |
| K_L6 | GAAATTGTGTTGACACAGTCTCCAG (SEQ ID |
| | NO: 183) |
| JK4_R | tatattccttaattaagttattctactcacGTTTGATCT |
| | CCACCTTGGTCCCT (SEQ ID NO: 184) |

TABLE 14
Variable domain primers (5′ to 3′) for 2D1.A3.D12

| | |
|---|---|
| H1_03 | CAGGTCCAGCTTGTGCAGTCTG (SEQ ID NO: 185) |
| G½_VH_R | TGGAGGCTGAGGAGACGGTGAC (SEQ ID NO: 186) |
| K_O12 | GACATCCAGATGACCCAGTCTCC (SEQ ID |
| | NO: 187) |
| JK1_R | tatattccttaattaagttattctactcacGTTTGATTT |
| | CCACCTTGGTCCCT (SEQ ID NO: 188) |

TABLE 15
Variable domain primers (5′ to 3′) for 14G9.B8.B4

| | |
|---|---|
| H1_03 | CAGGTCCAGCTTGTGCAGTCTG (SEQ ID NO: 189) |
| G½_VH_R | TGGAGGCTGAGGAGACGGTGAC (SEQ ID NO: 190) |
| K_A27 | GAAATTGTGTTGACGCAGTCTCCAG (SEQ ID |
| | NO: 191) |
| JK4_R | tatattccttaattaagttattctactcacGTTTGATCT |
| | CCACCTTGGTCCCT (SEQ ID NO: 192) |

### Example 4

Human Anti-CD44 Antibodies Block Binding of Hyaluronic Acid (HA) to CD44

Human anti-CD44 antibodies were evaluated for their ability to inhibit the interaction between HA (Sigma, Cat. No. H5388) and human CD44 protein (SEQ ID NO:3) as described in EXAMPLE 2.

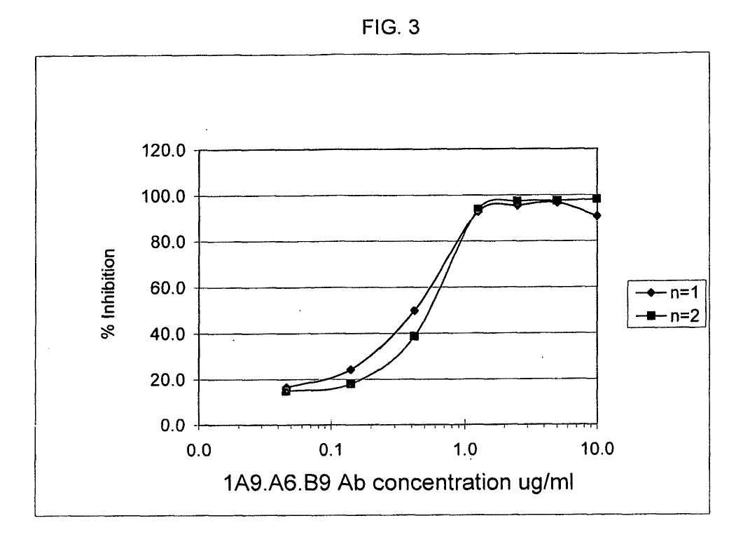

Binding assays were conducted in 96 well ELISA assay plates (Immunolux HB Maxisorp 96-well plates, Nunc Cat. No. 442-404). On day one, 100 μl of rooster comb HA diluted in 50 mM Nabicarb buffer, pH 9.6 at 2.5 mg/ml was added to assay wells on the plate and incubate at 4° C. overnight. Approximately, 24 hours later the HA coated plates were washed four times using 300 μl of PBS buffer with 0.05% Tween-20 (Sigma, Cat. No. P1379). The plates were then blocked by adding 200 μl of 3% BSA in PBS to each well and incubated for 2 hours at 37° C. The blocked plates were then washed with PBS with 0.05% Tween-20. In a separate 96 well polypropylene plate (Falco, Cat. No. 351190), anti-CD44 antibodies 1A9.A6.B9; 2D1.A3.D12; 14G9.B8.B4; and 10C8.2.3 were diluted in PBS with 1% BSA at various concentrations was mixed with human CD44-Ig fusion protein at a final concentration of 0.6 μg/ml in a 50 μl volume. The mixture was incubated at room temperature for 60 minutes and then transferred to the HA-coated plates and incubate at room temperature for one hour. The plates were washed by PBS with 0.05% Tween-20. Anti-human IgG-HRP (Amersham Biosciences, Piscataway, N.J., State, Cat. No. NA933) diluted 1:500 in 1% BSA (to detect CD44-Ig that is bound to HA) were added to each wells and incubate at room temperature. The plates were then washed again and 50 μl of TMB (TMB microwell peroxidase substrate, KPL, 52-00-02) was added and incubate for about 10 minutes. The reactions were then stopped with 50 μl of stop solution and OD₄₅₀ values were measured on a plate reader. FIG. 3 illustrates the graphic depiction of CD44 antibody 1A9.A6.B9 blocking the interaction between HA and CD44-Ig fusion protein. TABLE 16 shows the IC50's of the anti-CD44 antibodies.

TABLE 16

| Antibody Clone | IC50 (μg/mL) | |
|---|---|---|
| 1A9.A6.B9 | 0.41 ± 0.03 | (n = 3) |
| 2D1.A3.D12 | 0.33 ± 0.01 | (n = 2) |
| 14G9.B8.B4 | 0.43 ± 0.01 | (n = 3) |
| 10C8.2.3 | 0.64 | (n = 2) |
| IM7 | 1.85 ± 0.35 | (n = 9) |
| 515 | 0.32 | (n = 1) |

### Example 5

### Determination of Binding Constants of Anti-CD44 Monoclonal Antibodies

We conducted another in vitro assay to demonstrate the binding affinity of the antibodies of the invention to CD44.

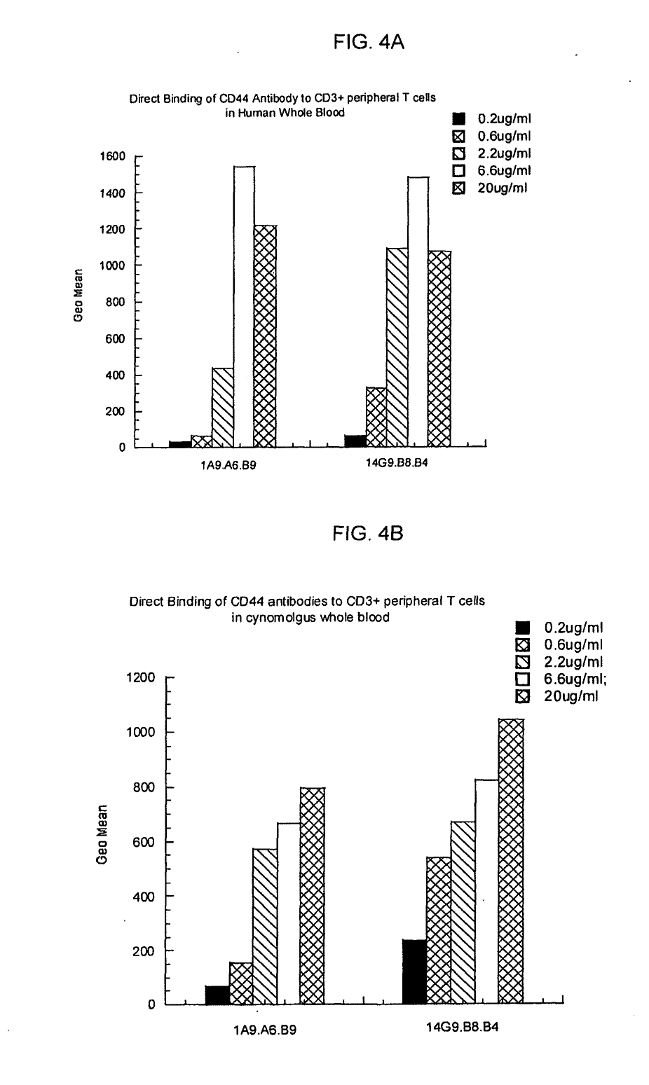

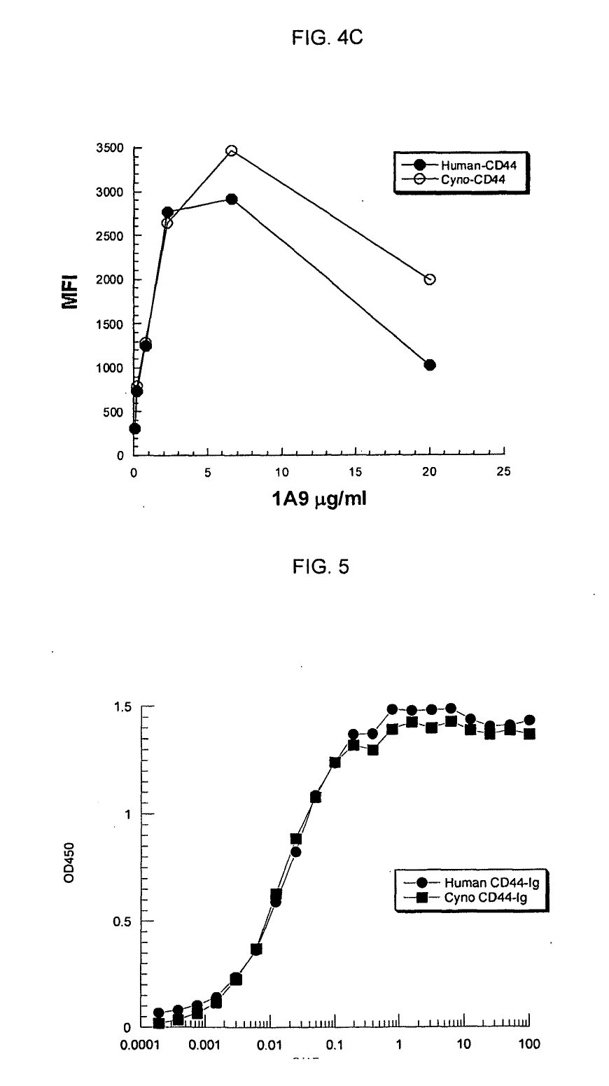

Binding studies demonstrated that anti-CD44 Abs bind to CD44 on transfected cells in an equilibrium binding analysis has a binding constant of 0.98 μg/mL (6.8 nM, see FIGS. 4 A-C, specifically FIG. 4C). The 300-19 cells which were transduced with retroviral vector encoding human CD44 protein and were washed two times by PBS. The 300-19 cells were then resuspended in FACS buffer [PBS, (Sigma, Cat. No. D-8537; 0.02% Azide (Sigma, Cat. No. S-2000); 5 μg/ml cytochalasin B, (Sigma, Cat. No. C-6762) and 2% Fetal Bovine Serum (Gibco, City, State, Cat. No. 16140-071)] at cell density of 1×10⁶ cells/ml. Transferred 400 μl of CD44 expression 300-19 cells (2×10⁵/400 μl) into Nunc-Immuno tubes, (VWR, Cat No. 443990), added 5 μl anti-hu IgG FITC (Jackson, Cat. No. 109-095-098) and 1A9.A6.B9 at various concentrations and incubated tubes on shaker plate (Thermolyne, rotomix type 48200) for 3 hours at room temperature with continued shaking. After 3 hours the cells were washed twice with FACS buffer. The cells were resuspended into 250 μl of 1% paraformaldehyde, (Electron Microscopy Science, Ft. Washington, Pa., Cat. No. 15710). The tubes were read using Becton Dickinson FACSCalibur and the data was analyzed using CellQuest Pro (Becton Dickinson). (See FIG. 4C).

Anti-CD44 antibodies also bind to human CD44 and cyno CD44 proteins expressed on peripheral CD3+ T cells. (see FIGS. 4A and 4B). Specifically, human peripheral blood was collected from normal human volunteers in vacutainer tubes with heparin (Becton Dickinson, Cat No. 366480). Added 100 μl of collected human blood to Nunc-Immuno tubes (VWR Cat No. 443990). Added anti-CD44 Abs into each tubes to achieve final concentrations from 0.2 μg/ml to 20 μg/ml. Thereafter added 10 μl of anti-CD3-PerCP (BD Pharmingen, Cat. No. 347344) and 10 μl of anti-CD14-APC (BD Pharmingen, Cat No. 555399) to each tube. Incubated for 30 minutes on ice, followed by centrifugation at 1200 rpm for 10 minutes, and removed supernatant. Added 100 μl of FACS wash buffer (PBS-Sigma D8537; 0.02% azide, Sigma Cat. No. S2002 and 2% fetal bovine serum, Gibco Cat. No. 16140-071) as well as added 50 μl/well of secondary FITC labeled goat anti human IgG Fc specific antibody (Jackson Cat. No. 109-095-098) at 1:100 fold dilution. Incubated for 25 minutes at 4° C. in dark. After 25 minute incubation added 2 mls of FACS lysing solution (BD Pharmingen, diluted 1:10 in water), vortexed and again incubated for 10 minutes at room temperature. After 10 minute incubation, centrifuged at 1200 rpm for 10 minutes and remove the supernatant, washed cells with FACS wash buffer, followed by centrifugation and removal of the supernatant. Cells were then fixed with 250 ml of 1% of paraformaldehyde (Electron Microscope Science, Ft Washington, Pa. Cat. No. 15710) and read using Becton Dickson FAGS Calibur, and analyzed using CellQuest Pro (Becton Dickinson).

### ELISA Binding Studies:

ELISA binding studies demonstrated that 1A9.A6.B9 binds to human and cyno CD44-Ig fusion protein coated on 96 well plates (see FIG. 5). To start the assay, 50 μl of CD44-Ig fusion protein at 1 μg/ml in PBS was added to a 96-well assay plate (Immuno Maxisorp plate, Nunc. Cat. No. 442-404) and incubated overnight at 4° C. Next day, the plates were washed four times with PBS, 0.05% Tween-20. The plates were then blocked by 3% BSA in PBS for 2 hours at 37° C., 200 μl per well and washed again. Anti CD44 antibody 1A9.A6.A9 was diluted at various concentrations with PBS and 1% BSA, and was added to the plates and incubated for one hour at room temperature. Plates were washed and 50 μl of anti-human kappa light chain-HRP antibody (Amersham Bioscience, Cat. No. NA 933), diluted 1:2000 in 1% BSA in PBS were added to each well and incubated at room temperature for another hour. Plates were washed again, and 50 μg/ml of TMB microwell peroxidase substrate (Cat. No. KPL S2-00-02)) were added and incubated for 5 to 10 minutes. ELISA reactions were stopped with stop solution and OD450 values were measured by a plate reader.

### BIAcore Binding Studies:

Surface plasmon resonance was used to measure the molecular interaction on a CM5 sensor chip coated with human CD44-Fc fusion protein (12 μg/ml, 10 mM Acetate, pH 4.0) on the surface. Anti-CD44 Abs at concentration of 5, 3, 2, 1, 0.5 and 0.25 μg/ml were screened by direct method. Human IgG1 and IgG2 standards were used to check nonspecific and background binding. Initial portion of the association and dissociation phase of the curves are used to calculate affinity and rate constants and is reported in TABLE 17.

TABLE 17
Biacore binding data for Anti-CD44 Antibodies

| Anti-CD44 | Affinity K_{D} × | Off rate k_{off} × |
|---|---|---|
| antibody | 10⁻⁹ (M) | 10⁻⁴ 1/s |
| 1A9.A6.A9 | 0.6 | 5.76 |
| 2D1.A3.D12 | 2.01 | 6.53 |
| 14G9.B8.B4 | 4.88 | 52.9 |

### Example 6

### Anti-CD44 Monoclonal Antibodies Block Inflammatory Cytokine Production from Human Peripheral Blood Mononuclear Cells (PBMC)

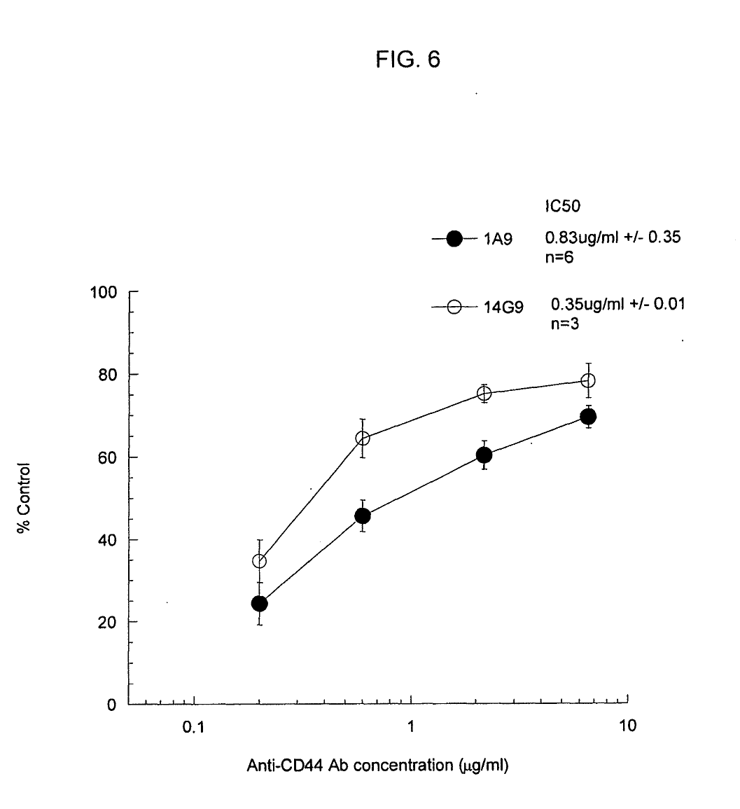

Anti-CD44 antibodies were also assessed for their ability to block IL-1β release stimulated by HA (Sigma, Cat. No. H5388) from purified human PBMC (see FIG. 6). Human peripheral blood was collected from normal human volunteers in vacutainer tubes with heparin (Becton Dickinson, Cat. No. 366480). PBMCs were isolated using Sigma Accuspin tubes (Sigma, Cat. No. A7054) according to the manufacture's instructions. The purified cells were washed two times with RPMI 1640 (Gibco, Cat. No. 11875-093) and resuspended at 5×10⁶ in RPMI and added to a 96 well assay plate (Costar, Cat. No. 3596),100 μl PBMCs per well. The human PBMCs were then stimulated by HA (Sigma, Cat. No. H1751) in the presence of various concentrations of anti-CD44 antibodies 1A9.A6.B9; 2D1.A3.D12; 14G9.B8.B4; and 10C8.2.3 in RPMI. Specifically, 100 μl of HA stock solution (10 μg/ml in RPMI) was mixed with PBMCs and 20 μl of anti-CD44 antibodies 1A9.A6.B9; 2D1.A3.D12; 14G9.B8.B4; and 10C8.2.3 at various concentrations. The assay plates were incubated for 24 hours at 37° C. in humidified atmosphere (Narco 6300 CO₂ incubator). The plates were then centrifuged for 10 minutes at 1200 rpms. Supernatants were then removed from each well and measured by IL-1β ELISA according to manufacture protocol (IL-1β Quantikine ELISA kit, R&D Cat. No.DLB50). (See, TABLE 18).

TABLE 18
Anti-CD44 monoclonal Ab in IL-1β release assay
using human purified PBMC stimulated by HA

| Antibody Clone | IC50 (μg/mL) | |
|---|---|---|
| 1A9.A6.A9 | 0.83 ± 0.61 | (n = 6) |
| 2D1.A3.D12 | 0.23 ± 0.23 | (n = 1) |
| 14G9.B8.B4 | 0.35 ± 0.02 | (n = 3) |
| 10C8.2.3 | 0.40 | (n = 1) |
| IM7 | 1.62 ± 0.93 | (n = 4) |
| 515 | 1.46 | (n = 1) |

### Example 7

### Anti CD44 Monoclonal Antibodies Block Cytokine Production from Human Peripheral T Cells

Anti-CD44 monoclonal antibodies blocked IL-2 and IFN-γ production from human peripheral T cells stimulated by anti-CD3 and anti-CD28 antibodies. Human peripheral blood was collected from normal human volunteers in vacutainer tubes with heparin (Becton Dickinson, Cat. No. 366480). The blood was then mixed with equal volume of anti-CD3 (UCTH1, R&D, Cat. No. MAB100) and anti-CD28 antibody (R&D, Cat. No. AF-342-PB) diluted in PBS in Falcon polypropylene tubes (Falcon Cat. No. 2059). The final concentrations of the anti-CD3 and anti-CD28 antibodies were about 1 μg/ml and 10 ng/ml respectively. In a 96 well polystyrene plates (Costar, Cat. No. 3596), 10 μl of anti-CD44 antibodies 1A9.A6.B9; 2D1.A3.D12; and 14G9.B8.B4, at various concentrations, were added to each well and then mixed with 200 μl of human whole blood that pre-mixed with anti-CD3 and anti-CD28 antibodies, incubated at 37° C. for 24 hours. Serum was removed and tested by IFN-γ and IL-2 ELISA assays (R&D, Cat. Nos. DIF50 and D2050, respectively). (See TABLE 19 below).

TABLE 19
Anti-CD44 Abs Block IL-2 and IFN-γ Release from Human Peripheral
Blood Activated by Anti-CD3 and Anti-CD28 Antibodies

| | IC 50 (μg/ml) | | | |
|---|---|---|---|---|
| Anti-CD-44 Antibodies | IL-2 | | IFN-γ | |
| 1A9.A6.A9 | 0.58 ± 0.21 | (n = 6) | 2.46 ± 1.46 | n = 7 |
| 14G9 B8.34 | Inactive | (n = 10) | Inactive | (n = 10) |
| 2D1 WT/H38Q | 0.46 ± 0.06 | (n = 5) | 2.45 ± 2.2 | (n= 2) |

### Example 8

### Reduction of Surface Expression of CD44

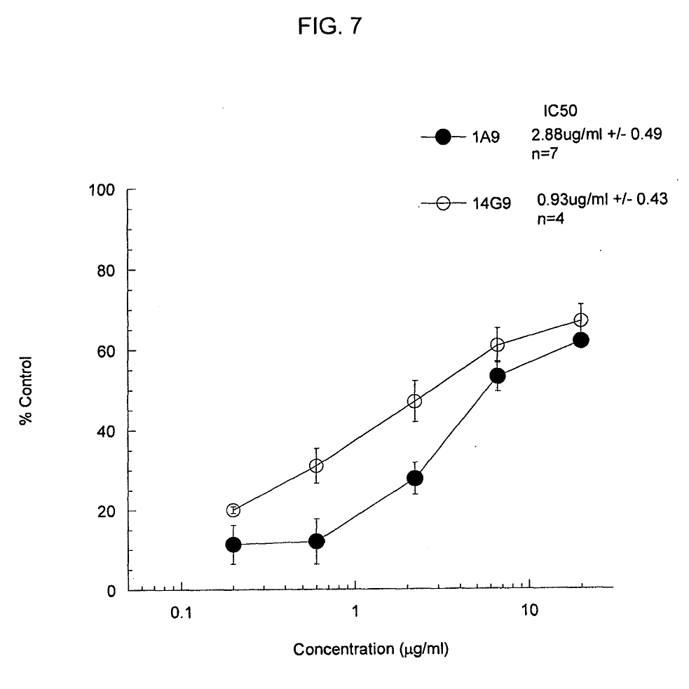

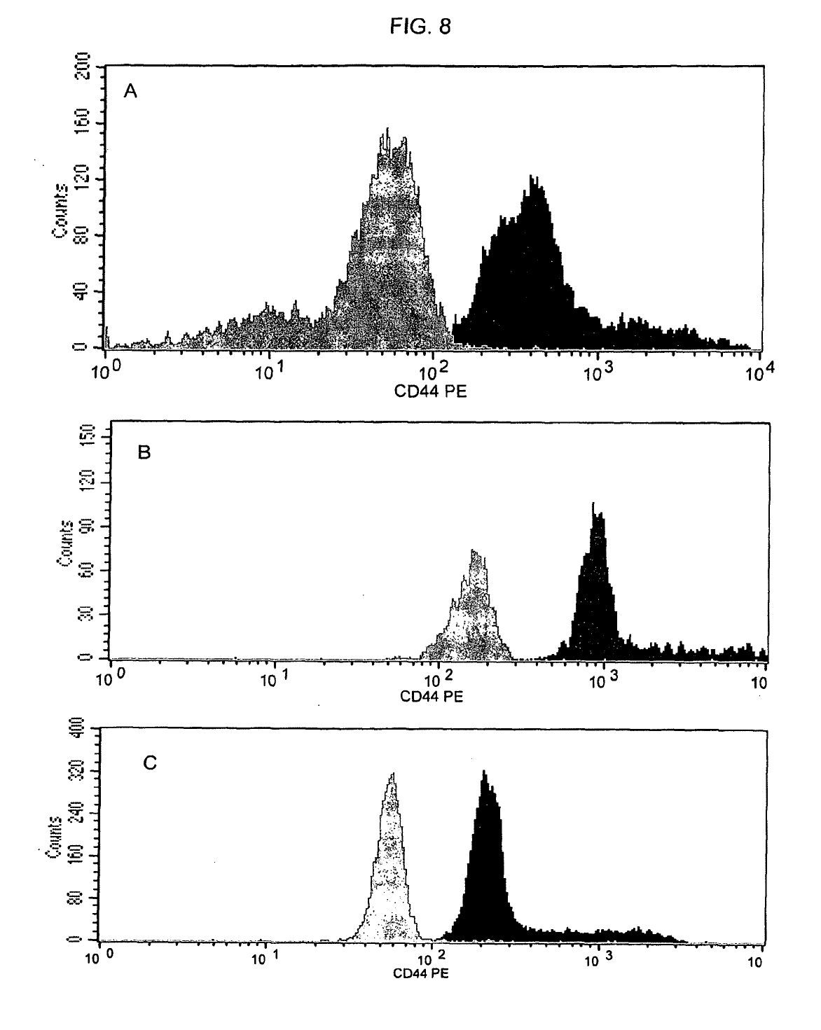

Flow cytometry (FAGS) analyses were performed to detect a reduction of surface expression of CD44 by the anti-CD44 antibody. We incubated each anti-CD44 antibody with human whole blood under in vitro condition for approximately 12 hours, and detected reduced CD44 surface expression level on human peripheral leukocytes (see FIG. 7). Ten μl of anti-CD44 antibodies 1A9.A6.B9; 2D1.A3.D12; 14G9.B8.B4; and 10C8.2.3 antibodies at various concentrations were added to a 96 well flat bottom polystyrene assay plate (Costar, Cat. No. 3596). Human peripheral blood was collected from normal human volunteers in vacutainer tubes with heparin (Becton Dickinson, Cat. No. 366480). One hundred μl per well of human blood was then mixed with anti-CD44 Ab, and incubate at 37° C. for 24 hours in humidified atmosphere (Narco 6300 CO₂ incubator). Twenty pls of CD44 detection antibody, G-44-26-PE (BD PharMingen, Franklin Lakes, N.J., Cat. No. 555479), 10 μl of anti-CD3-perCP antibody (BD PharMingen, Cat. No. 347344) and 10 μl of anti-CD14-APC antibody (BD PharMingen, Cat. No. 555399) were then added to the wells, and kept on ice for 30 to 40 minutes in the dark. One hundred μl of blood was then removed from the plates and transferred to nunc-immuno tubes (VWR, West Chester, Pa., Cat. No. 443990) and 2 mls FACS lysing solution (BD Pharmingen Cat. No. 349202) was added at a dilution of 1:10 water, vortexed and set aside for 10 minutes at room temperature. After 10 minutes the blood was centrifuged at 1200 rpms for 10 minutes and the cells were washed with FACS wash buffer (PBS, 0.02% azide, Sigma, Cat. No. S2002 and 2% Fetal Bovine Serum (Gibco, Cat. No. 16140-071). The blood was centrifuged again at 1200 rpms for 10 minutes, and washed with FACS wash buffer. The cells were then fixed with 250 ml of 1% paraformaldehyde (Electron Microscopy Science, Cat. No. 15710) and the tubes were read using FACS calibur and data were analyzed using Cellquest software. (See TABLES 20 and 21). FIG. 8 shows the FACS results at a concentration of 10 μg/ml 1A9.A6.B9 antibody for (a) Lymphocytes, (b) monocytes, and (c) PMNs. The 1A9.A6.B9 antibody result is shown in gray and the baseline expression in black.

TABLE 20
Anti-CD44 Abs reduce CD44 surface expression on
human and cynomolgus peripheral CD3+ T cells

| | |
|---|---|
| Human peripheral | Cynomolgus peripheral |
| CD3+ T cells | CD3+ T cells |
| IC50 (μg/ml) | IC50 (μg/ml) |

| 1A9.A6.B9 | 2.8 ± 1.3 | (n = 6) | 0.82 ± 0.16 | (n = 3) |
|---|---|---|---|---|
| 14G9.B8.B4 | 0.93 ± 0.85 | (n = 4) | 0.39 ± 0.18 | (n = 3) |
| 2D1.A3.D12 | >20 | (n = 4) | >20 | (n = 4) |
| 10C8.2.3 | >20 | (n = 2) | — | |
| IM7 | 1.5 ± 0.71 | (n = 2) | 9.9 | (n = 1) |

| | |
|---|---|
| 515 | Inactive* |

*less than 40% inhibition at 20 μg/ml

TABLE 21
1A9.A6.B9 Reduces CD44 Expression on Leukocytes in Human
and Cynomolgus Peripheral Blood (See FIGS. 8A-8C)

IC50 (μg/ml)

| Leukocytes | Human | Cynomolgus |
|---|---|---|
| T cells | 2.6 ± 1.0 (n = 10) | 1.1 ± 0.5 (n = 5) |
| Monocytes | 3.3 ± 0.3 (n = 3) | N.R. |
| B cells | 2.4 ± 1.1 (n = 4) | N.T. |

N.R. = No response
N.T. = not tested

### Example 9

### Single Dose In Vivo Study of Anti-CD44 Antibody 1A9.A6.B9 Induces a Dose Dependent Decrease in CD44 Expression on Peripheral CD3+T Cells in Cynomolgus Monkeys

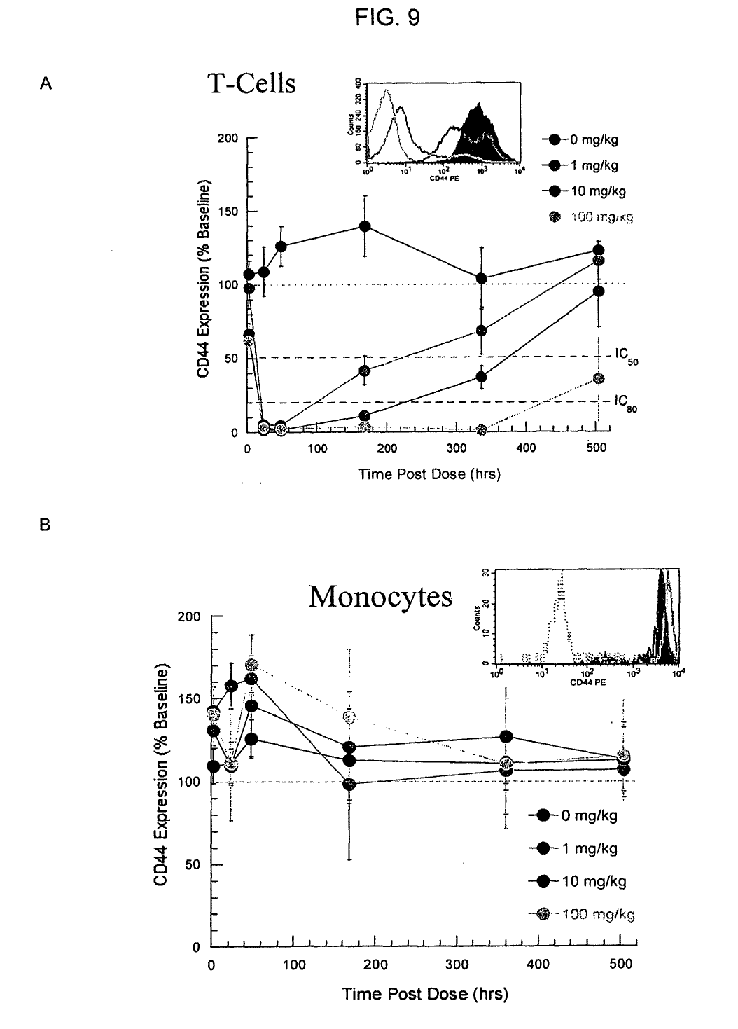

Reduction of CD44 surface expression from lymphocytes (see FIG. 9A) and monocytes (see FIG. 9B) induced by anti-CD44 antibodies, was examined by the administration of a single intravenous dose of 1A9.A6.B9 (10 mg/ml in 25 mM sodium acetate, 140 mM NaCl, 0.2 mg/ml polysorbate-80, PH5.5) at 1, 10 and 100 mg/kg (2 male and 2 female animals per dose group) to cynomolgus monkeys supplied by Charles River Primates, BRF (Bio Research Facility, House Texas). Blood samples (−2 ml) were collected by femoral venipuncture from fasted monkeys twice pre-treatment, and 2, 24, 48, 168, 336 and 504 hours post dose for three-color (CD3+, CD14+, CD44+) flow cytometric analysis.

For detecting the CD44 expression by FACS assay, 100 μl of peripheral blood was mixed with either combination of 20 μl of CD14-FITC (Clone M5E2, BD-Pharm Cat. No. 67509), 20 μl of CD3-PerCP (BD-Pharm, Cat. No. 13043) and 10 μl of CD44-PE (IM7, BD-Pharm Cat. No. 8900), or combination of 20 μl of CD14-FITC (Clone M5E2, BD-Pharm Cat. No. 67509), 20 μl of CD3-PerCP (BD-Pharm, Cat. No. 13043) and 10 μl of Rt IgG2b-PE (IM7, BD-Pharm Cat. No. 60254). The antibody was mixed with the blood using a vortex at a low moderate speed for 1 second. The blood with antibodies were incubated for 20 to 30 minutes at 4° C., adding 1.5 ml of 1:10 FACS lyse solution (B.D. Pharmingen, San Diego, Calif.) to each tube. Each tube was mixed on the vortex at low/moderate speed for 1-3 seconds. The tubes were incubated at room temperature for approximately 12 minutes in the dark. To ensure complete lysis the opacity of each tube was checked, and an additional 500 μl of FAGS lyse was added to tubes that appeared cloudy. An additional 2 ml of BD stain buffer (BD PharMingen, San Diego, Calif., Cat. No. 55465C) was added; the tubes were recapped and mixed by invertion of the tubes. The tubes were then placed in a swing bucket and centrifuged at 250×g for 6-7 minutes at room temperature. The cell pellets were washed by stain buffer. One hundred μl of the cytofix buffer (PBS with 4% w/v paraformaldehyde) was added to the cells. The samples were kept at 4° C. in the dark until they were acquired on the FACSCalibur. One hundred ul of the cytofix buffer (PBS with 4% W/V paraformaldehyde) were added to the cells. The samples were stored in the cytofix buffers at 4° C. in the dark. One hundred ul of PBS was added to all the tubes before the cells were analyzed on a FACSCalibur. A total of 20,000 events on gated lymphocytes were collected.

### Example 10

### Epitope Classification Studies

Competition binding analysis was performed using BIAcore™.

### BIAcore Epitope Mapping Experiments:

Epitope mapping of CD44 antibodies, 1A9.A6.B9, 2D1.A3.D12 and 14G9.B8.B4 was performed by running competition assay on BIAcore™ (see TABLE 22 for antibody concentrations and TABLE 23 epitope map). The Biosensor biospecific interaction analysis instrument (BIAcore 2000) with surface plasmon resonance was used to measure molecular interactions on a CM5 sensor chip. Changes in the refractive indices between two media, glass and carboxymethylated dextran, caused by the interaction of molecules to the dextran side of the sensor chip, was measured and reported as changes in arbitrary reflectance units (RU) as detailed in the manufacturer's application notes.

The carboxymethylated dextran surface of a flow cell on a sensor chip was activated by derivatization with 0.05 M N-hydroxysuccinimide mediated by 0.2 M N-ethyl-N′-(dimethylaminopropyl) carbodiimide for 7 minutes. CD44-Ig at a concentration of 30 μg/ml, in 10 mM Na acetate, pH 3.5, was manually injected into the flow cell at a rate 5 μl/min and covalently immobilized to the flow cell surface with the desired amount of RU's. Deactivation of unreacted N-hydroxysuccinimide esters was performed using 1M ethanolamine hydrochloride, pH 8.5. Following immobilization, the flow cells were cleaned of any unreacted or poorly bound material with 5 regeneration injections of 5 μl of 50 mM NaOH until a stable baseline was achieved. Flow cell 2 measured approximately. 62 RU and flow cell 3 measured approximately 153 RU. For flow cell 1, the activated blank surface, 35 μl of 10 mM Na acetate buffer was injected during immobilization in place of antigen. Flow cell 4 contained approximately 200 RU's of immobilized CTLA4-Ig, an irrelevant antigen control.

The epitope mapping experiment was carried out using running/diluent buffer (HBS-EP). The flow rate was 5 μl/min and the instrument temperature was 20° C. Following binding of each pair of antibodies, the flow cell surface was then regenerated to baseline using a 5 μl injection of 50 mM NaOH. Purified antibodies in running buffer were diluted to 30 μg/ml and injected in a volume of 25 μl.

The flow cell surface was saturated with a primary antibody and was immediately followed with an injection of a second antibody. Binding of the second antibody was then assessed as “binding”, “not binding” or “partially binding” to the immobilized CD44-Ig surface. After a binding assessment is made, the surface is regenerated and the same primary antibody is reinjected followed by the next antibody in the panel. This injection scheme is continued until all antibodies in the panel have been assessed for their binding to CD44-Ig. Another antibody is chosen as the primary and the other antibodies are assessed as the secondary antibodies binding to CD44-Ig. Specifically, when the anti-CD44 antibody 14G9.B8.B4, was tested as the primary antibody, it was co-injected with a second antibody as the off-rate for 14G9.B8.B4 is fast with respect to binding.

After all antibodies have been tested as primary injections against all the antibodies in the panel, a reduced matrix combining similar binding patterns into one epitope group is prepared. A topological map can then be drawn according to the reduced matrix. The binding matrix can be interpreted in terms of a topological surface map of the antigen, CD44-Ig, showing interference between different epitopes. Such a map shows functional relationships only and does not necessarily bear any correspondence to the actual physical structure of the antigen surface.

BIAcore competition binding analysis showed that the epitope recognized by mAbs 1A9.A6.B9 and 14G9.B8.B4 overlap with the epitope recognized by antibody 515. Moreover, the BIAcore™ study showed that mAbs 1A9.A6.B9 and 14G9.B8.B4 did not overlap with antibody IM7.

TABLE 22

| Antibodies | Final Concentration |
|---|---|
| IM7 (BD Bioscience, Frankilin Lakes, NJ, | 1.0 mg/ml |
| Cat. No. 553134) | |
| 515 (BD Bioscience, Cat. No. 550990) | 1.0 mg/ml |
| 1A9.A6.B9 | 1.5 mg/ml |
| 14G9.B9.B4 | 1.0 mg/ml |

TABLE 23
Competition Epitope Mapping of CD44 antibodies

| | IM7 (BD) | 1A9.A6.B9 | 515 (BD) | 14G9.B8.B4 | Rmax |
|---|---|---|---|---|---|
| IM7 (BD) | X | ◯ | ◯ | ◯ | 233 |
| 1A9.A6.B9 | ◯ | X | X | X | 226 |
| 515 (BD) | ◯ | X | X | X | 170 |
| 14G9.B8.B4 | ◯ | X | X | X | 144 |

X = competition observed
◯ = competition not observed

### Example 11

### Selectivity of Anti-CD44 Antibody

We measured the binding affinity of CD44 antibody verses a lymphatic vessel endothelia hyauronan receptor 1 protein (LYVE-1) (R&D, Cat. No. 2089-Ly) and found the anti-CD44 antibody has more than 100-fold selectivity for CD44 over LYVE-1 (see TABLE 24). A 96 well ELISA plate (Immuno Maxisorp plate, Nunc Cat. No. 442-404) was coated with 50 ng CD44-Ig fusion protein or LYVE-1 and incubated overnight at 4° C. The plates were then washed by PBS, 0.05% Tween-20 and blocked by 3% BSA in PBS for two hours at room temperature. The anti-CD44 antibodies or anti-LYVE-1 antibody (R&D, Cat. No. AF 2089) was diluted in 1% BSA in PBS at various concentrations and added to the plates. The ELISA plates were incubated at room temperature for 1.5 hours. Plates were washed and 50 μl of either anti-human kappa light chain-HRP antibody (Bethyl, Cat. No. A80-115P.6) for anti-CD44 antibody or anti-goat IgG-HRP for anti-LYVE-1 antibody (Cappel/ICN, Cat. No. 55363), diluted 1:2000 in 1% BSA in PBS were added to each well and incubated at room temperature for another hour. Plates were washed again, and 50 μg/ml of TMB were added and incubated for 5 to 10 minutes. ELISA reactions were stopped with stop solution and OD450 values were measured by a plate reader.

TABLE 24
Selectivity of anti-CD44 antibodies

| Antibodies | CD44-Ig (EC50 μg/ml) | LYVE-1 (EC50 μg/ml) |
|---|---|---|
| 1A9.A6.B9 | 0.011 | no cross reactivity |
| | | at 10 μg/ml |
| 2D1.A3.D12 | 0.024 | no cross reactivity |
| | | at 10 μg/ml |
| 14G9.B8.B4 | 0.018 | no cross reactivity |
| | | at 10 μg/ml |
| LYVE-1 Ab | >>10 | 0.1 |

### Example 12

### Binding Competition Studies of MEM-85 and 1A9.A6.B9 Anti-CD44 Antibodies

We conducted FACS studies to determine whether human anti-CD44 antibodies in accordance with the invention bind to the same or distinct site on the CD44 molecule as commercially available anti-CD44 antibody MEM-85 (Caltag Laboratories, Burlingame, Calif., Cat. No. MHCD4404-4).

We have performed the FACS based CD44 competition binding assay either used CD3+ human peripheral T cells and 300-19 cells transduced with human CD44 molecule on a retroviral vector. Human peripheral blood was collected from normal human volunteers in vacutainer tubes with heparin (Becton Dickinson, Cat No. 366480).

### Human Peripheral T Cell FAGS Study:

Human peripheral blood was collected from normal human volunteers in vacutainer tubes with heparin (Becton Dickinson, Cat No. 366480). Added 100 μl of collected human blood to Nunc-Immuno tubes (VWR Cat. No. 443990), thereafter added 10 μl of anti-CD44 Ab 1A9.A6.B9 into each tube to achieve final concentrations from 0.2 μg/ml to 20 μg/ml. The tubes were incubated for 5 minutes on ice. After the 5 minute incubation, added 20 μl of CD44 detection Ab (MEM-85, Cat. No. MHCD4404-4) and anti-CD3-PerCP (BD Pharmingen, Cat. No. 347344), 10 μl of anti-CD14-APC (BD Pharmingen, Cat. No. 555399), and 10 ml anti-CD4-APC (BD Pharmingen Cat. No. 555349) to each tube and kept on ice for 30 to 40 minutes, in the dark. Added 2 mls of FACS lysing solution (BD Pharmingen, Cat. No. 349202, diluted 1:10 in water), vortexed and incubated for 10 minutes at room temperature. Washed cells with FACS wash buffer, (PBS Sigma Cat. No. D8537, 0.02% azide, Sigma Cat. No. S2002 and 2% fetal calf serum, Gibco Cat. No. 16140-071) followed by centrifuge and removed the supernatant. Cells were then fixed with 250 μl of 1% of paraformaldehyde (Electron Microscope Science, Ft Washington, Pa. Cat. No. 15710). Tubes were read using Becton Dickson FACS Calibur and analyze the data using CellQuest Pro (Becton Dickinson).

300-19 Cells Transduced with Human CD44 Molecule FACS Study:

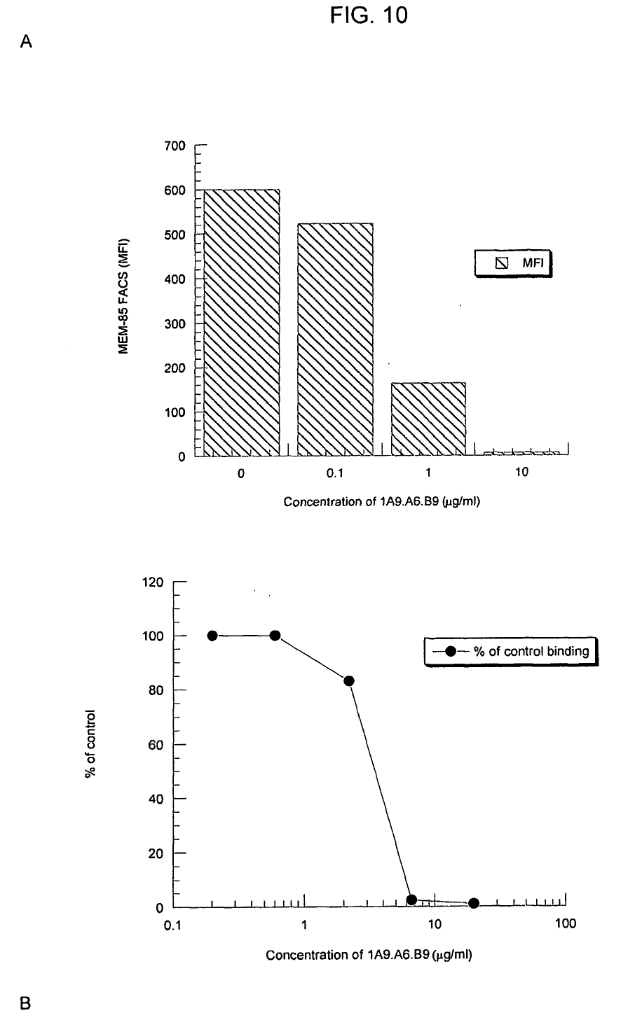

One hundred mls of 300-19 cells at 10⁶ cell/ml were added to Nunc-Immuno tubes (VWR Cat. No. 443990). Cells were then mixed with 10 μl of anti-CD44 Ab to achieve final concentration from 0 to 10 μg/ml (see FIG. 10A) and incubated on ice for 5 minutes. After the incubation, added 20 μl of CD44 detection Ab MEM-85 (Caltag Laboratories, Burlingame, Calif., Cat. No. MHCD4404-4) to the tubes and incubated the cells on ice for 30 to 40 minutes. Wash with FACS wash buffer (PBS Sigma D8537, 0.02% azide, Sigma S2002 and 2% fetal calf serum, Gibco Cat No. 16140-071). Centrifuged at 12000 rpm for 10 minutes and eliminated the supernatant. Fixed cells with 250 ml of 1% paraformaldehyde (Electron Microscope Science, Ft Washington, Pa. Cat. No. 15710). Tubes were read using Becton Dickson FACS Calibur and analyze the data using CellQuest Pro (Becton Dickinson).

FACS competition binding analysis showed that the epitope recognized by mAb 1A9.A6.B9 overlaps with the epitope recognized by the MEM-85 antibody, which has been mapping to the LINK domain on CD44 molecule. Bajorath, J. et al., (1998) JBC, 273:338-343 (See FIGS. 10A-B).

### Example 13

Two lyophilized (freeze-dried) formulations of 1A9.A6.B9 (HIS lyo & CIT Lyo) were prepared as per the Table 25, below. Formulations contained 20 mg/mL 1A9.A6.B9, histidine or citrate buffer, polysorbate 80, EDTA, and trehalose dihydrate. A liquid formulation (HIS liquid) was also prepared, as shown in Table 25 below (1A9.A6.B9). The composition of the liquid formulation was: 10 mg/mL 1A9.A6.B9, 20 mM histidine buffer, 0.2 mg/mL polysorbate 80, 0.05 mg/mL EDTA, 84 mg/mL trehalose dihydrate and 0.1 mg/mL L-methionine.

TABLE 25
Formulation components for 1A9.A6.B9 liquid and lyo formulations

| | 1A9.A6.B9 | | Histidine | Citrate | PS80 | Trehalose | Sucrose | EDTA | Methionine |
|---|---|---|---|---|---|---|---|---|---|
| Formulation | (mg/ml) | pH | (mM) | (mM) | (mg/mL) | (mg/mL) | (mg/mL) | (mg/mL) | (mg/mL) |
| (HIS liquid) | 10 | 5.5 | 20 | — | 0.2 | 84 | — | 0.05 | 0.1 |
| (HIS lyo) | 20 | 5.5 | 20 | — | 0.2 | — | 80 | 0.05 | — |
| (CIT lyo) | 20 | 5.5 | — | 5 | 0.2 | — | 80 | 0.05 | — |

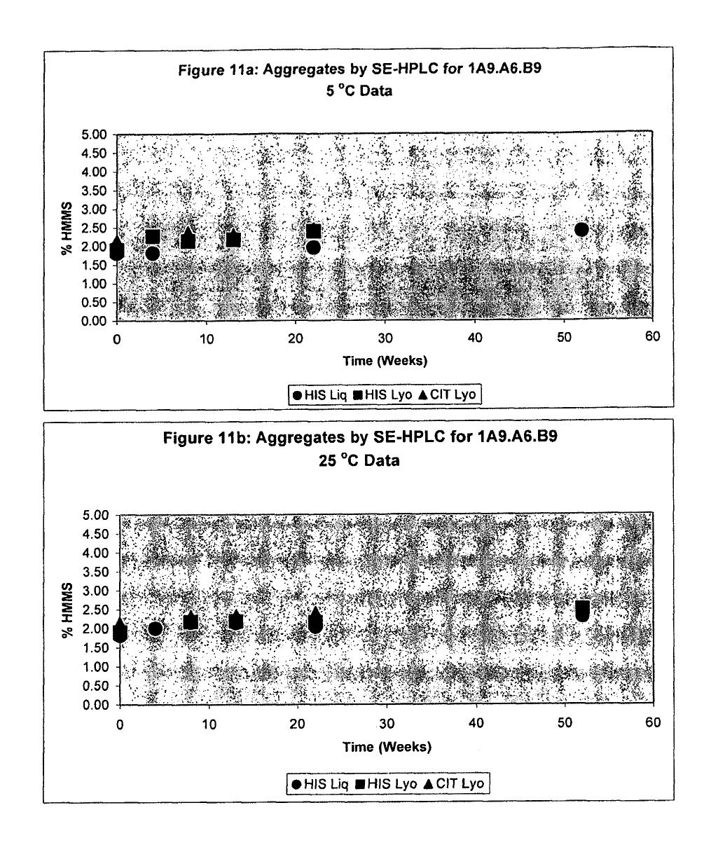

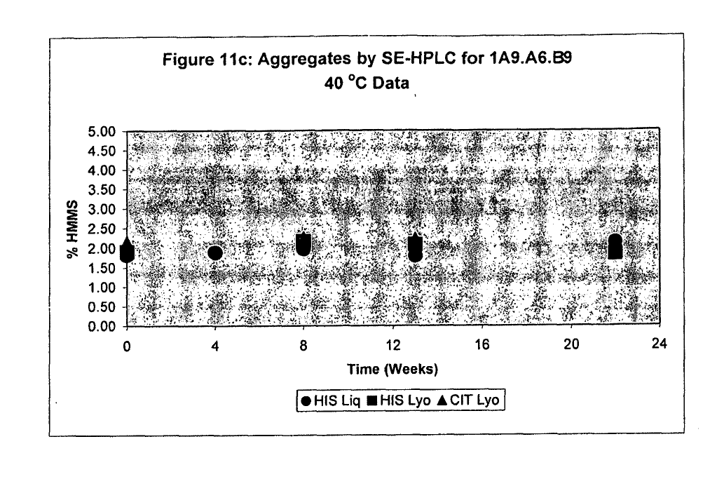

Each of the formulations prepared above were kept at 2-8° C. and accelerated stability conditions (25 and 40° C.) for 52 weeks (the lyophilized formulation containing citrate buffer was kept only 22 weeks). Samples were analyzed at 4, 8, 13, 22 and 52 weeks. At each time point, samples were analyzed visually for presence of particulates, change in color, and clarity. pH measurements were also conducted. Presence of aggregates was monitored by SE-HPLC. All formulations tested remained visually clear, colorless and free of particles and did not show any significant change in pH. In addition, better than 97% mAb monomer recovery (<3% aggregate formation) was obtained for all formulations of Table 25, and the liquid formulation which was tested as a control after being subjected to storage at 2-8° C. and 25° C. for 52 weeks as well as at 40° C. for 22 weeks (FIG. 11 a, b & c) as measured by SE-HPLC using 2 columns in series (GS SW3000XL and GS SW2000XL) using a mobile phase that is 200 mM Phosphate buffer at pH 7.0. The flow rate was kept at 0.7 mL/min with a run time of 40 min.

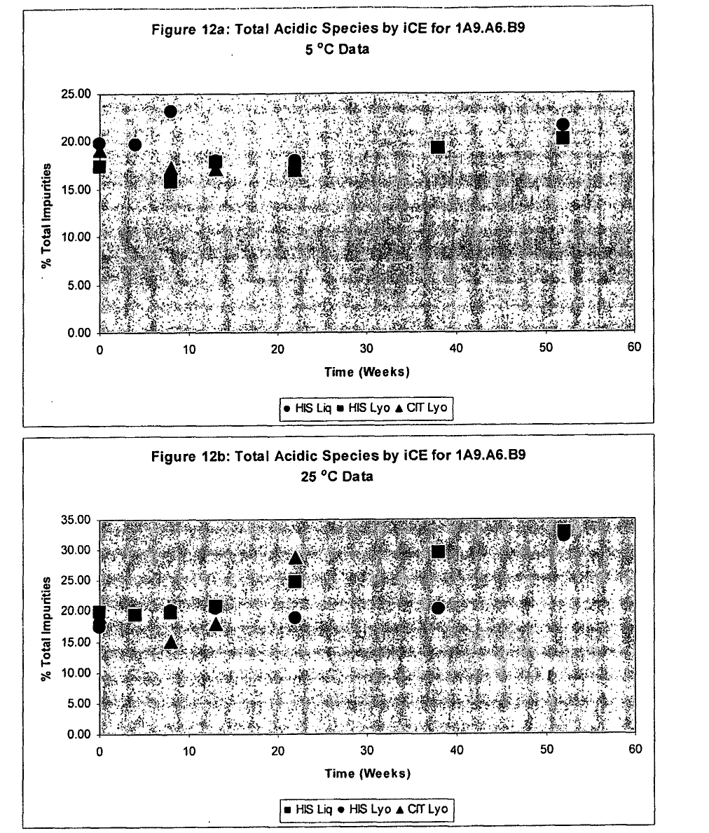

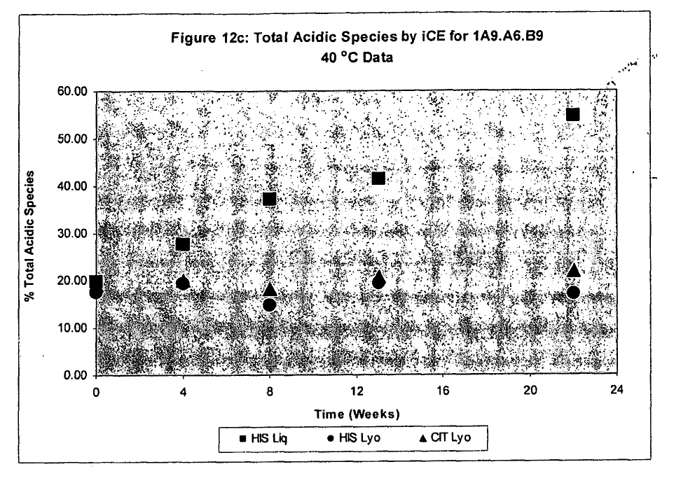

Each of the formulations was analyzed by imaging capillary iso-electric focusing (iCE) to evaluate the formation of 1A9.A6.B9 charge variants (acidic, parent, and basic species) after 52 weeks of refrigerated storage (2-8° C.) and under accelerated temperature conditions (25° C. for 52 weeks as well as at 40° C. for 22 weeks). The separation of these charged species was done within a capillary and the visualization and quantification of these species using a UV detector and CCD camera. The results of the iCE assay (acidic species) are illustrated in FIG. 12 a, b & c. The results demonstrate that all the formulations had similar acidic species formation after storage at 2-8° C. and 25° C. for 52 weeks. At 40° C. the lyophilized formulations reported lower acidic species formation than the control.

Each of the formulations was also analyzed by SDS-PAGE to evaluate the formation of higher and lower size variants of the mAb after 52 weeks of refrigerated storage (2-8° C.) and under accelerated stability conditions (25° C. for 52 weeks as well as at 40° C. for 22 weeks). This method provides a good measure of mAb purity, including levels of clip formation and aggregate formation over time. The results of the SDS-PAGE assay are illustrated in Table 26, 27 & 28.

Each of the formulations were also analyzed to evaluate the formation of methionine oxidation at the methionine-256 position on the heavy chain after 52 weeks of refrigerated storage (2-8° C.) and under accelerated stability conditions (25° C. for 52 weeks or 40° C. for 22 weeks). The monoclonal antibody products were digested with Lys-C and a methionine-containing peptide fragment and its respective oxidized form were monitored. Tables 26, 27 & 28 show the results of the methionine oxidation assay.

Each of the formulations were also analyzed to evaluate the relative bioactivity after 52 weeks of refrigerated storage (2-8° C.) and under accelerated stability conditions (25° C. for 52 weeks as well as at 40° C. for 22 weeks). The results of the bioactivity assay are illustrated in Table 26, 27 & 28.

TABLE 26
Stability Data obtained at 5 C.

| | Time-Point | | | | | |
|---|---|---|---|---|---|---|
| | 22 weeks | | | 52 weeks | | |
| Formulation | HIS-liquid | HIS-lyo | CIT-lyo | HIS-liquid | HIS-lyo | CIT-lyo |

SDS-PAGE

| | | | | | | |
|---|---|---|---|---|---|---|
| % Impurites >50K | 0.8 | 0.5 | 0.7 | 0.6 | 0.6 | Not |
| | | | | | | analyzed |
| % Impurites 25K-50K | 0 | 0 | 0 | 0 | 0 | Not |
| | | | | | | analyzed |
| % Impurites <25K | 0 | 0 | 0 | 0 | 0 | Not |
| | | | | | | analyzed |
| Total % | 0.8 | 0.5 | 0.7 | 0.6 | 0.6 | Not |
| Impurites | | | | | | analyzed |

Oxidation

| | | | | | | |
|---|---|---|---|---|---|---|
| met-256 | 3.1 | 3.2 | 3.4 | Not | Not | Not |
| | | | | analyzed | analyzed | analyzed |

REDUCED CGE

| | | | | | | |
|---|---|---|---|---|---|---|
| % Purity | 98.8 | 98.7 | 98.8 | 98.7 | 98.8 | Not |
| | | | | | | analyzed |
| % Fragment | 1.2 | 1.3 | 1.2 | 1.3 | 1.2 | Not |
| | | | | | | analyzed |
| BioAssay | 95% | Not | Not | Not | Not | Not |
| | | analyzed | analyzed | analyzed | analyzed | analyzed |

TABLE 27
Stability Data obtained at 25 C.

| | Time-Point | | | | | |
|---|---|---|---|---|---|---|
| | 22 weeks | | | 52 weeks | | |
| Formulation | HIS-liquid | HIS-lyo | CIT-lyo | HIS-liquid | HIS-lyo | CIT-lyo |

SDS-PAGE

| | | | | | | |
|---|---|---|---|---|---|---|
| % Impurites >50K | 1.0 | 0.6 | 2.4 | 1.4 | 0.3 | Not |
| | | | | | | analyzed |
| % Impurites 25K-50K | 0.9 | 0.1 | 0.2 | 0.6 | 0 | Not |
| | | | | | | analyzed |
| % Impurites <25K | 0.1 | 0.0 | 0.1 | 0.0 | 0.0 | Not |
| | | | | | | analyzed |
| Total % | 2.0 | 0.7 | 2.7 | 2.0 | 0.3 | Not |
| Impurites | | | | | | analyzed |

Oxidation

| | | | | | | |
|---|---|---|---|---|---|---|
| met-256 | Not | Not | Not | Not | Not | Not |
| | analyzed | analyzed | analyzed | analyzed | analyzed | analyzed |
| BioAssay | Not | Not | Not | Not | Not | Not |
| | analyzed | analyzed | analyzed | analyzed | analyzed | analyzed |

TABLE 28
Stability Data obtained at 40 C.

| | Time-Point | | |
|---|---|---|---|
| | 22 weeks | | |
| Formulation | HIS-liquid | HIS-lyo | CIT-lyo |

SDS-PAGE

| | | | |
|---|---|---|---|
| % Impurites >50K | 2.0 | 0.6 | 1.1 |
| % Impurites 25K-50K | 4.1 | 0.2 | 0 |
| % Impurites <25K | 1.5 | 0 | 0 |
| Total % | 7.6 | 0.9 | 1.1 |
| Impurites | | | |

Oxidation

| | | | |
|---|---|---|---|
| met-256 | 8.8 | 3.5 | 3.4 |

CGE (Reduced)

| | | | |
|---|---|---|---|
| % Purity | 91.2 | 98.9 | 98.9 |
| % Fragment | 8.3 | 1.1 | 1.1 |
| BioAssay | 86 | 73 | 92 |

### Example 14

### Binding Affinity of 1A9.A6.B9 for Human and Cynomologus Monkey CD44 by Biacore™ Analysis

Another BIAcore™ analysis was conducted to demonstrate the binding affinity of the 1A9.A6.B9 antibody to human and cynmologous CD44. Human CD44-Ig (55 RU, 86 RU) and cyno CD44-Ig (99 RU, 116 RU) were immobilized to CM-5 chips at a concentration of 10 ug/ml in 10 mM Na Acetate pH 3.5. Varying concentrations of 1A9.A6.B9, (100 ug/ml to 0.1 ug/ml in half-log dilutions) were flowed over the chip at a flow rate of 5 ul/minute. The chip was then regenerated with 50 mM NaOH and washed with HBS-EP (BIAcore 22-0512-44). Analysis was carried out on the Biacore 2000. Data was analyzed using BIAEvaluation™ software (n=2).

TABLE 29
Kinetic analysis of 1A9.A6.B9

| CD44 Ig | Kd (×10⁻³) (1/s) | K_{D} (pM) |
|---|---|---|
| Human | 0.39 | 51 (n = 3) |
| Cynomolgus | 0.53 | 150 (n = 3) |

All references cited in this specification, including without limitation all papers, publications, patents, patent applications, presentations, texts, reports, manuscripts, brochures, books, internet postings, journal articles, periodicals, product fact sheets, and the like, one hereby incorporated by reference into this specification in their entireties. The discussion of the references herein is intended to merely summarize the assertions made by their authors and no admission is made that any reference constitutes prior art and Applicants' reserve the right to challenge the accuracy and pertirency of the cited references.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appendant claims.

We claim:

1. An isolated human antibody or antigen-binding portion thereof that specifically binds human CD44, comprising a heavy chain variable (V_{H}) domain amino acid sequence comprising a CDR1, CDR2, and CDR3 region selected from the group consisting of:
a) a V_{H} CDR1 as set forth in SEQ ID NO:17, a V_{H} CDR2 as set forth in SEQ ID NO:19, and a V_{H} CDR3 as set forth in SEQ ID NO:21;
b) a V_{H} CDR1 as set forth in SEQ ID NO:53, a V_{H} CDR2 as set forth in SEQ ID NO:55, and a V_{H} CDR3 as set forth in SEQ ID NO:57;
c) a V_{H} CDR1 as set forth in SEQ ID NO:89, a V_{H} CDR2 as set forth in SEQ ID NO:91, and a V_{H} CDR3 as set forth in SEQ ID NO:93; and
d) a V_{H} CDR1 as set forth in SEQ ID NO:125, a V_{H} CDR2 as set forth in SEQ ID NO:127, a V_{H} CDR3 as set forth in SEQ ID NO:129.

2. The isolated human antibody or antigen-binding portion according to claim 1 further comprising a light chain variable (V_{L}) domain amino acid sequence comprising a CDR1, CDR2, and CDR3 region selected from the group consisting of:
a) a V_{L} CDR1 as set forth in SEQ ID NOs:23, a V_{L} CDR2 as set forth in SEQ ID NOs:25, and a V_{L} CDR3 as set forth in SEQ ID NO:27;
b) a V_{L} CDR1 as set forth in SEQ ID NOs:59, a V_{L} CDR2 as set forth in SEQ ID NOs:61, and a V_{L} CDR3 as set forth in SEQ ID NO:63;
c) a V_{L} CDR1 as set forth in SEQ ID NOs:95, a V_{L} CDR2 as set forth in SEQ ID NOs:97, and a V_{L} CDR3 as set forth in SEQ ID NO:99; and
d) a V_{L} CDR1 as set forth in SEQ ID NOs:131, a V_{L} CDR2 as set forth in SEQ ID NOs:133, and a V_{L} CDR3 as set forth in SEQ ID NO:135.

3. The isolated antibody or antigen-binding portion thereof according to claim 2 comprising a V_{H} CDR1 as set forth in SEQ ID NO:17, a V_{H} CDR2 as set forth in SEQ ID NO:19, a V_{H} CDR3 as set forth in SEQ ID NO:21, a V_{L} CDR1 as set forth in SEQ ID NO:23, a V_{L} CDR2 as set forth in SEQ ID NO:25, and a V_{L} CDR3 as set forth in SEQ ID NO:27.

4. The isolated antibody or antigen-binding portion thereof according to claim 2 comprising a V_{H} CDR1 as set forth in SEQ ID NO:89, a V_{H} CDR2 as set forth in SEQ ID NO:91, a V_{H} CDR3 as set forth in SEQ ID N093, a V_{L} CDR1 as set forth in SEQ ID NO:95, a V_{L} CDR2 as set forth in SEQ ID NO:97, and a V_{L} CDR3 as set forth in SEQ ID NO:99.

5. The antibody or antigen-binding portion thereof according to claim 1 wherein the V_{H} domain amino acid sequence is selected from the group consisting of: SEQ ID NOs: 11, 47, 83 and 119, or differs from any one of SEQ ID NOs: 11, 47, 83 and 119 by having a conservative amino acid substitution.

6. The antibody or antigen-binding portion thereof according to claim 1, comprising a V_{H} domain that is at least 95% identical in amino acid sequence to any one of SEQ ID NOs:11, 47, 83 and 119.

7. The antibody or antigen-binding portion thereof according to claim 2 wherein the V_{L} domain amino acid sequence is selected from the group consisting of SEQ ID NOs:15, 51, 87 and 123, or differs from any one of SEQ ID NOs: 15, 51, 87 and 123 by having a conservative amino acid substitution.

8. The antibody or antigen-binding portion thereof according to claim 2, comprising a V_{L} domain that is at least 95% identical in amino acid sequence to any one of SEQ ID NOs:15, 51, 87 and 123.

9. The antibody or antigen-binding portion thereof according to claim 7, wherein the (V_{H}) domain amino acid sequence and the (V_{L}) domain amino acid sequence are selected from the group consisting of:
a) a V_{H} domain as set forth in SEQ ID NO:11 and a V_{L} domain as set forth in SEQ ID NO:15;
b) a V_{H} domain as set forth in SEQ ID NO:47 and a V_{L} domain as set forth in SEQ ID NO:51;
c) a V_{H} domain as set forth in SEQ ID NO:83 and a V_{L} domain as set forth in SEQ ID NO:87; and
d) a V_{H} domain as set forth in SEQ ID NO:119 and a V_{L} domain as set forth in SEQ ID NO:123.

10. The antibody or antigen-binding portion thereof according to claim 9 comprising a V_{H} domain as set forth in SEQ ID NO:11 and a V_{L} domain as set forth in SEQ ID NO:15.

11. The antibody or antigen-binding portion thereof according to claim 9 comprising a V_{H} domain as set forth in SEQ ID NO:83 and a V_{L} domain as set forth in SEQ ID NO:87.

12. An isolated human antibody that specifically binds to CD44 comprising a heavy chain amino acid sequence and a light chain amino acid sequence selected from the group consisting of:
a) a heavy chain as set forth in SEQ ID NO:9, and a light chain as set forth in SEQ ID NO:13;
b) a heavy chain as set forth in SEQ ID NO:45, and a light chain as set forth in SEQ ID NO:49;
c) a heavy chain as set forth in SEQ ID NO:81, and a light chain as set forth in SEQ ID NO:85; and
d) a heavy chain as set forth in SEQ ID NO:117, and a light chain as set forth in SEQ ID NO:121.

13. The isolated human antibody or antigen-binding portion thereof according to claim 12 comprising a heavy chain as set forth in SEQ ID NO:9 and a light chain as set forth in SEQ ID NO:13.

14. The isolated human antibody or antigen-binding portion thereof according to claim 12 comprising a heavy chain as set forth in SEQ ID NO:9 and a light chain as set forth in SEQ ID NO:13.

15. The antibody according to claim 9 that is an IgG.

16. The antibody according to claim 15 wherein the IgG is an IgG₂.

17. A pharmaceutical composition comprising the antibody or antigen-binding portion according to claim 1 and optionally a pharmaceutically acceptable carrier.

18. A method of treating, preventing or alleviating the symptoms of a CD44-mediated disorder in a subject in need thereof with an anti-CD44 antibody or antigen-binding portion thereof, comprising the step of administering to the subject an effective amount of an antibody or antigen binding portion thereof according to claim 1.

19. A method of treatment according to claim 17, wherein the CD44 mediated disorder is an inflammatory or autoimmune disease.

20. A method of treatment according to claim 18, wherein the disease is selected from the group consisting of rheumatoid arthritis, juvenile rheumatoid arthritis, atherosclerosis, granulmatous diseases, multiples sclerosis, asthma, Crohn's disease, ankylosing spondylitis, psoriatic arthritis, plaque psoriasis and cancer.

21. An isolated nucleic acid molecule comprising a nucleotide sequence that encodes the heavy chain or an antigen-binding portion thereof and/or the light chain or an antigen-binding portion thereof of an antibody according to claim 1.

22. A vector comprising the nucleic acid molecule of claim 21 wherein the vector optionally comprises an expression control sequence operably linked to the nucleic acid molecule

23. A host cell comprising the vector according to claim 22.

24. A hybridoma cell line that produces a human antibody according to claim 1, wherein the hybridoma is selected from the group consisting of 2D1.A3.D12 (ATCC No. PTA-6929) (LN 15920), 1A9.A6.B9 (ATCC No. PTA-6927) (LN 15922) and 14G9.B8.B4 (ATCC No. PTA-6928) (LN 15921).

25. The hybridoma cell line according to claim 24, wherein the hybridoma is 1A9.A6.B9 (ATCC No. PTA-6927) (LN 15922).


SEQUENCE LISTING (200 sequences)

SEQ ID NO: 1 - protein, 361 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 2 - DNA, 1086 bp, Homo sapiens
atggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaa

SEQ ID NO: 3 - protein, 499 aa, Homo sapiens
QIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEDPGGGGGRLVPRGFGTGDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1578 bp, Homo sapiens
atgcccatggggtctctgcaaccgctggccaccttgtacctgctggggatgctggtcgcttcctgcctcggaactagtcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcatgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaagatcccggcggcggcggcggccgcctggttcctcgtggcttcggtaccggagatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggattggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctttacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 248 aa, Macaca fascicularis
QIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPE

SEQ ID NO: 6 - DNA, 744 bp, Homo sapiens
cagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaa

SEQ ID NO: 7 - protein, 361 aa, Macaca fascicularis
MDKFWWHAAWGLCLLQLSLAQIDLNITCRFAGVFHVEKNGRYSISRTEAADLCKAFNSTLPTMAQMEKALSVGFETCRYGFIEGHVVIPRIQPNSICAANHTGVYILTSNTSQYDTYCFNASAPPKEDCTSVTDLPNAFDGPITITIVNPDGTRYIKKGEYRTNPEDIYPSNPTDDDVSSGSSSERSSTSGGYIFHTFSTAHPIPDEDGPWITDSTDRIPATRDQDAFYPSGGSHTTHGSESAGHSHGSQEGGANTTSGPVRTPQIPEWLIILASLLALALILAVCIAVNSRRRCGQKKKLVINSGNGAVDDRKPSGLNGEASKSQEMVHLVNKEPSETPDQFMTADETRNLQNVDMKIGV

SEQ ID NO: 8 - DNA, 1086 bp, Macaca fascicularis
atggacaagttttggtggcacgcagcctggggactctgcctcttgcagctgagcctggcgcagatcgatttgaatataacctgccgctttgcgggtgtattccacgtggagaaaaatggtcgctacagcatctctcggacggaggctgctgacctctgcaaggctttcaatagcaccttgcccacaatggcccagatggagaaagctctgagcgtcggatttgagacctgcaggtacgggttcatagaagggcacgtggtgattccccggattcagcccaactccatctgtgcagcaaaccacacaggggtgtacatcctcacgtccaacacctcccagtatgacacatactgcttcaatgcttcagctccacctaaagaagattgtacatcagtcacagacctgcccaatgcctttgatggaccaattaccataactattgttaaccccgatggcactcgctatatcaagaaaggagaatacagaacgaatcctgaagacatctaccccagcaaccctactgacgatgacgtgagcagcggatcctccagtgaaaggagcagcacttcgggaggttacatctttcacaccttttctactgcacaccccatcccagacgaagacggtccctggatcaccgacagcacagacagaatccctgctaccagagaccaagatgcattctaccccagtggggggtcccataccactcatggatctgaatcagctggacactcacatgggagtcaagaaggtggggcaaacacaacctctggtcctgtaaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggatgatagaaagccaagtggactcaatggagaggccagcaagtctcaggaaatggtgcatttggtgaacaaggagccatcagaaactccagaccagtttatgacagctgatgagacaaggaacctgcagaacgtggacatgaagattggggtgtaa

SEQ ID NO: 9 - protein, 447 aa, Artificial Sequence
  Description of Artificial Sequence Synthetic construct
QVQLVESGGGVVQPGRSLRLSCAASGFTFSSYGMHWVRQAPGKGLEWVAVIWYDGSNKFYADSVKGRFTISRDNSKNTLYLQMNSLRAEDTAVYYCARRSDYRGYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - DNA, 1341 bp, Artificial Sequence
  Description of Artificial Sequence Synthetic construct
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaattctatgcagactccgtgaagggccgattcaccatctccagagacaattccaagaacacgctgtatctgcaaatgaacagcctgagagccgaggacacggctgtgtattactgtgcgaggagaagtgactacaggggctactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 11 - protein, 121 aa, Artificial Sequence
  Description of Artificial Sequence Synthetic construct
QVQLVESGGGVVQPGRSLRLSCAASGFTFSSYGMHWVRQAPGKGLEWVAVIWYDGSNKFYADSVKGRFTISRDNSKNTLYLQMNSLRAEDTAVYYCARRSDYRGYYGMDVWGQGTTVTVSS

SEQ ID NO: 12 - DNA, 363 bp, Artificial Sequence
  Description of Artificial Sequence Synthetic construct
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaattctatgcagactccgtgaagggccgattcaccatctccagagacaattccaagaacacgctgtatctgcaaatgaacagcctgagagccgaggacacggctgtgtattactgtgcgaggagaagtgactacaggggctactacggtatggacgtctggggccaagggaccacggtcaccgtctcctca

SEQ ID NO: 13 - protein, 214 aa, Artificial Sequence
  Description of Artificial Sequence Synthetic construct
EIVLTQSPATLSLSPGERATLSCRASQSVINYLAWYQQKPGQAPRLLIYDASNRASGIPARFSGSGSGTDFTLTISSLEPEDFAVYYCQQRRNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 642 bp, Artificial Sequence
  Description of Artificial Sequence Synthetic construct
gaaattgtgttgacacagtctccagccaccctgtctttgtctccaggggaaagagccaccctctcctgcagggccagtcagagtgttatcaactacttagcctggtaccaacagaaacctggccaggctcccaggctcctcatctatgatgcatccaacagggcctctggcatcccagccaggttcagtggcagtgggtctgggacagacttcactctcaccatcagcagcctagagcctgaagattttgcagtttattactgtcagcagcgtcgcaactggccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 107 aa, Artificial Sequence
  Description of Artificial Sequence Synthetic construct
EIVLTQSPATLSLSPGERATLSCRASQSVINYLAWYQQKPGQAPRLLIYDASNRASGIPARFSGSGSGTDFTLTISSLEPEDFAVYYCQQRRNWPLTFGGGTKVEIK

SEQ ID NO: 16 - DNA, 322 bp, Artificial Sequence
  Description of Artificial Sequence Synthetic construct
gaaattgtgttgacacagtctccagccaccctgtctttgtctccaggggaaagagccaccctctcctgcagggccagtcagagtgttatcaactacttagcctggtaccaacagaaacctggccaggctcccaggctcctcatctatgatgcatccaacagggcctctggcatcccagccaggttcagtggcagtgggtctgggacagacttcactctcaccatcagcagcctagagcctgaagattttgcagtttattactgtcagcagcgtcgcaactggccgctcactttcggcggagggaccaaggtggagatcaaac

SEQ ID NO: 17 - protein, 5 aa, Artificial Sequence
  Description of Artificial Sequence Synthetic peptide
SYGMH

SEQ ID NO: 18 - DNA, 15 bp, Artificial Sequence
  Description of Artificial Sequence Synthetic oligonucleotide
agctatggcatgcac

SEQ ID NO: 19 - protein, 17 aa, Artificial Sequence
  Description of Artificial Sequence Synthetic peptide
VIWYDGSNKFYADSVKG

SEQ ID NO: 20 - DNA, 51 bp, Artificial Sequence
  Description of Artificial Sequence Synthetic oligonucleotide
gttatatggtatgatggaagtaataaattctatgcagactccgtgaagggc

SEQ ID NO: 21 - protein, 12 aa, Artificial Sequence
  Description of Artificial Sequence Synthetic peptide
RSDYRGYYGMDV

SEQ ID NO: 22 - DNA, 36 bp, Artificial Sequence
  Description of Artificial Sequence Synthetic oligonucleotide
agaagtgactacaggggctactacggtatggacgtc

SEQ ID NO: 23 - protein, 11 aa, Artificial Sequence
  Description of Artificial Sequence Synthetic peptide
RASQSVINYLA

SEQ ID NO: 24 - DNA, 33 bp, Artificial Sequence
  Description of Artificial Sequence Synthetic oligonucleotide
agggccagtcagagtgttatcaactacttagcc

SEQ ID NO: 25 - protein, 7 aa, Artificial Sequence
  Description of Artificial Sequence Synthetic peptide
DASNRAS

SEQ ID NO: 26 - DNA, 21 bp, Artificial Sequence
  Description of Artificial Sequence Synthetic oligonucleotide
gatgcatccaacagggcctct

SEQ ID NO: 27 - protein, 9 aa, Artificial Sequence
  Description of Artificial Sequence Synthetic peptide
QQRRNWPLT

SEQ ID NO: 28 - DNA, 27 bp, Artificial Sequence
  Description of Artificial Sequence Synthetic oligonucleotide
cagcagcgtcgcaactggccgctcact

SEQ ID NO: 29 - protein, 30 aa, Artificial Sequence
  Description of Artificial Sequence Synthetic peptide
QVQLVESGGGVVQPGRSLRLSCAASGFTFS

SEQ ID NO: 30 - DNA, 90 bp, Artificial Sequence
  Description of Artificial Sequence Synthetic oligonucleotide
caggtgcagctggtggagtctgggggaggcgtggtccagcctgggaggtccctgagactctcctgtgcagcgtctggattcaccttcagt

SEQ ID NO: 31 - protein, 23 aa, Artificial Sequence
  Description of Artificial Sequence Synthetic peptide
EIVLTQSPATLSLSPGERATLSC

SEQ ID NO: 32 - DNA, 69 bp, Artificial Sequence
  Description of Artificial Sequence Synthetic oligonucleotide
gaaattgtgttgacacagtctccagccaccctgtctttgtctccaggggaaagagccaccctctcctgc

SEQ ID NO: 33 - protein, 14 aa, Artificial Sequence
  Description of Artificial Sequence Synthetic peptide
WVRQAPGKGLEWVA

SEQ ID NO: 34 - DNA, 42 bp, Artificial Sequence
  Description of Artificial Sequence Synthetic oligonucleotide
tgggtccgccaggctccaggcaaggggctggagtgggtggca

SEQ ID NO: 35 - protein, 15 aa, Artificial Sequence
  Description of Artificial Sequence Synthetic peptide
WYQQKPGQAPRLLIY

SEQ ID NO: 36 - DNA, 45 bp, Artificial Sequence
  Description of Artificial Sequence Synthetic oligonucleotide
tggtaccaacagaaacctggccaggctcccaggctcctcatctat

SEQ ID NO: 37 - protein, 32 aa, Artificial Sequence
  Description of Artificial Sequence Synthetic peptide
RFTISRDNSKNTLYLQMNSLRAEDTAVYYCAR

SEQ ID NO: 38 - DNA, 96 bp, Artificial Sequence
  Description of Artificial Sequence Synthetic oligonucleotide
cgattcaccatctccagagacaattccaagaacacgctgtatctgcaaatgaacagcctgagagccgaggacacggctgtgtattactgtgcgagg

SEQ ID NO: 39 - protein, 32 aa, Artificial Sequence
  Description of Artificial Sequence Synthetic peptide
GIPARFSGSGSGTDFTLTISSLEPEDFAVYYC

SEQ ID NO: 40 - DNA, 96 bp, Artificial Sequence
  Description of Artificial Sequence Synthetic oligonucleotide
ggcatcccagccaggttcagtggcagtgggtctgggacagacttcactctcaccatcagcagcctagagcctgaagattttgcagtttattactgt

SEQ ID NO: 41 - protein, 11 aa, Artificial Sequence
  Description of Artificial Sequence Synthetic peptide
WGQGTTVTVSS

SEQ ID NO: 42 - DNA, 33 bp, Artificial Sequence
  Description of Artificial Sequence Synthetic oligonucleotide
tggggccaagggaccacggtcaccgtctcctca

SEQ ID NO: 43 - protein, 10 aa, Artificial Sequence
  Description of Artificial Sequence Synthetic peptide
FGGGTKVEIK

SEQ ID NO: 44 - DNA, 30 bp, Artificial Sequence
  Description of Artificial Sequence Synthetic oligonucleotide
ttcggcggagggaccaaggtggagatcaaa

SEQ ID NO: 45 - protein, 452 aa, Artificial Sequence
  Description of Artificial Sequence Synthetic construct
QVQLVQSGAEVKKPGASVKVSCKASGYIFTSYAMHWVRQAPGQRLEWMGWINAAIGSTKYSQKFQGRVTITRDTSASTAYMELSSLRSEDTAVYYCARDGWEDYYYHGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1356 bp, Artificial Sequence
  Description of Artificial Sequence Synthetic construct
caggtccaacttgtgcagtctggggctgaggtgaagaagcctggggcctcagtgaaggtttcctgcaaggcttctggatacatcttcactagctatgctatgcattgggtgcgccaggcccccggacaaaggcttgagtggatggggtggatcaacgctgccattggtagcacaaaatattcacagaagttccagggcagagtcaccattaccagggacacatccgcgagcacagcctacatggagctgagcagcctgagatctgaagacacggctgtgtattactgtgcgagagacgggtgggaggactactactaccacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - protein, 122 aa, Artificial Sequence
  Description of Artificial Sequence Synthetic construct
QVQLVQSGAEVKKPGASVKVSCKASGYIFTSYAMHWVRQAPGQRLEWMGWINAAIGSTKYSQKFQGRVTITRDTSASTAYMELSSLRSEDTAVYYCARDGWEDYYYHGMDVWGQGTTVTVSS

SEQ ID NO: 48 - DNA, 366 bp, Artificial Sequence
  Description of Artificial Sequence Synthetic construct
caggtccaacttgtgcagtctggggctgaggtgaagaagcctggggcctcagtgaaggtttcctgcaaggcttctggatacatcttcactagctatgctatgcattgggtgcgccaggcccccggacaaaggcttgagtggatggggtggatcaacgctgccattggtagcacaaaatattcacagaagttccagggcagagtcaccattaccagggacacatccgcgagcacagcctacatggagctgagcagcctgagatctgaagacacggctgtgtattactgtgcgagagacgggtgggaggactactactaccacggtatggacgtctggggccaagggaccacggtcaccgtctcctca

SEQ ID NO: 49 - protein, 214 aa, Artificial Sequence
  Description of Artificial Sequence Synthetic construct
DIQMTQSPSSVSASVGDRVTITCRASQGISSWLAWYQHKPGKAPKLLIYAASSLQSGVPSRFSGSGSGTDFTLTISSLQPEDFATYYCQQANNFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - DNA, 642 bp, Artificial Sequence
  Description of Artificial Sequence Synthetic construct
gacatccagatgacccagtctccatcttccgtgtctgcatctgtaggagacagagtcaccatcacttgtcgggcgagtcagggtattagtagctggttagcctggtatcagcataaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaataatttcccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 51 - protein, 107 aa, Artificial Sequence
  Description of Artificial Sequence Synthetic construct
DIQMTQSPSSVSASVGDRVTITCRASQGISSWLAWYQHKPGKAPKLLIYAASSLQSGVPSRFSGSGSGTDFTLTISSLQPEDFATYYCQQANNFPWTFGQGTKVEIK

SEQ ID NO: 52 - DNA, 322 bp, Artificial Sequence
  Description of Artificial Sequence Synthetic construct
gacatccagatgacccagtctccatcttccgtgtctgcatctgtaggagacagagtcaccatcacttgtcgggcgagtcagggtattagtagctggttagcctggtatcagcataaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaataatttcccgtggacgttcggccaagggaccaaggtggaaatcaaac

SEQ ID NO: 53 - protein, 5 aa, Artificial Sequence
  Description of Artificial Sequence Synthetic peptide
SYAMH

SEQ ID NO: 54 - DNA, 15 bp, Artificial Sequence
  Description of Artificial Sequence Synthetic oligonucleotide
agctatgctatgcat

SEQ ID NO: 55 - protein, 17 aa, Artificial Sequence
  Description of Artificial Sequence Synthetic peptide
WINAAIGSTKYSQKFQG

SEQ ID NO: 56 - DNA, 51 bp, Artificial Sequence
  Description of Artificial Sequence Synthetic oligonucleotide
tggatcaacgctgccattggtagcacaaaatattcacagaagttccagggc

SEQ ID NO: 57 - protein, 13 aa, Artificial Sequence
  Description of Artificial Sequence Synthetic peptide
DGWEDYYYHGMDV

SEQ ID NO: 58 - DNA, 39 bp, Artificial Sequence
  Description of Artificial Sequence Synthetic oligonucleotide
gacgggtgggaggactactactaccacggtatggacgtc

SEQ ID NO: 59 - protein, 11 aa, Artificial Sequence
  Description of Artificial Sequence Synthetic peptide
RASQGISSWLA

SEQ ID NO: 60 - DNA, 33 bp, Artificial Sequence
  Description of Artificial Sequence Synthetic oligonucleotide
cgggcgagtcagggtattagtagctggttagcc

SEQ ID NO: 61 - protein, 7 aa, Artificial Sequence
  Description of Artificial Sequence Synthetic peptide
AASSLQS

SEQ ID NO: 62 - DNA, 21 bp, Artificial Sequence
  Description of Artificial Sequence Synthetic oligonucleotide
gctgcatccagtttgcaaagt

SEQ ID NO: 63 - protein, 9 aa, Artificial Sequence
  Description of Artificial Sequence Synthetic peptide
QQANNFPWT

SEQ ID NO: 64 - DNA, 27 bp, Artificial Sequence
  Description of Artificial Sequence Synthetic oligonucleotide
caacaggctaataatttcccgtggacg

SEQ ID NO: 65 - protein, 30 aa, Artificial Sequence
  Description of Artificial Sequence Synthetic peptide
QVQLVQSGAEVKKPGASVKVSCKASGYIFT

SEQ ID NO: 66 - DNA, 90 bp, Artificial Sequence
  Description of Artificial Sequence Synthetic oligonucleotide
caggtccaacttgtgcagtctggggctgaggtgaagaagcctggggcctcagtgaaggtttcctgcaaggcttctggatacatcttcact

SEQ ID NO: 67 - protein, 23 aa, Artificial Sequence
  Description of Artificial Sequence Synthetic peptide
DIQMTQSPSSVSASVGDRVTITC

SEQ ID NO: 68 - DNA, 69 bp, Artificial Sequence
  Description of Artificial Sequence Synthetic oligonucleotide
gacatccagatgacccagtctccatcttccgtgtctgcatctgtaggagacagagtcaccatcacttgt

SEQ ID NO: 69 - protein, 14 aa, Artificial Sequence
  Description of Artificial Sequence Synthetic peptide
WVRQAPGQRLEWMG

SEQ ID NO: 70 - DNA, 42 bp, Artificial Sequence
  Description of Artificial Sequence Synthetic oligonucleotide
tgggtgcgccaggcccccggacaaaggcttgagtggatgggg

SEQ ID NO: 71 - protein, 15 aa, Artificial Sequence
  Description of Artificial Sequence Synthetic peptide
WYQHKPGKAPKLLIY

SEQ ID NO: 72 - DNA, 45 bp, Artificial Sequence
  Description of Artificial Sequence Synthetic oligonucleotide
tggtatcagcataaaccagggaaagcccctaagctcctgatctat

SEQ ID NO: 73 - protein, 32 aa, Artificial Sequence
  Description of Artificial Sequence Synthetic peptide
RVTITRDTSASTAYMELSSLRSEDTAVYYCAR

SEQ ID NO: 74 - DNA, 96 bp, Artificial Sequence
  Description of Artificial Sequence Synthetic oligonucleotide
agagtcaccattaccagggacacatccgcgagcacagcctacatggagctgagcagcctgagatctgaagacacggctgtgtattactgtgcgaga

SEQ ID NO: 75 - protein, 32 aa, Artificial Sequence
  Description of Artificial Sequence Synthetic peptide
GVPSRFSGSGSGTDFTLTISSLQPEDFATYYC

SEQ ID NO: 76 - DNA, 96 bp, Artificial Sequence
  Description of Artificial Sequence Synthetic oligonucleotide
ggggtcccatcaaggttcagcggcagtggatctgggacagatttcactctcaccatcagcagcctgcagcctgaagattttgcaacttactattgt

SEQ ID NO: 77 - protein, 11 aa, Artificial Sequence
  Description of Artificial Sequence Synthetic peptide
WGQGTTVTVSS

SEQ ID NO: 78 - DNA, 33 bp, Artificial Sequence; identical to SEQ ID NO: 42
  Description of Artificial Sequence Synthetic oligonucleotide

SEQ ID NO: 79 - protein, 10 aa, Artificial Sequence
  Description of Artificial Sequence Synthetic peptide
FGQGTKVEIK

SEQ ID NO: 80 - DNA, 30 bp, Artificial Sequence
  Description of Artificial Sequence Synthetic oligonucleotide
ttcggccaagggaccaaggtggaaatcaaa

SEQ ID NO: 81 - protein, 447 aa, Artificial Sequence
  Description of Artificial Sequence Synthetic construct
QVQLVQSGAEVKKPGASVKVSCKASGYTFTNYAMHWVRQAPGQRLEWMGWINTGNGNTKYSQKFQGRVTITRDTSASTAYMELSSLRSEDTAVYYCARFYSGSGSPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - DNA, 1341 bp, Artificial Sequence
  Description of Artificial Sequence Synthetic construct
caggtccagcttgtgcagtctggggctgaggtgaagaagcctggggcctcagtgaaggtttcctgcaaggcttctggatacaccttcactaactatgctatgcattgggtgcgccaggcccccggacaaaggcttgagtggatgggatggatcaacactggcaatggtaacacaaaatattcacagaagttccagggcagagtcaccattaccagggacacatccgcgagcacagcctacatggagctgagcagcctgagatctgaagacacggctgtgtattactgtgcgaggttttactctggttcggggagtccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 83 - protein, 117 aa, Artificial Sequence
  Description of Artificial Sequence Synthetic construct
QVQLVQSGAEVKKPGASVKVSCKASGYTFTNYAMHWVRQAPGQRLEWMGWINTGNGNTKYSQKFQGRVTITRDTSASTAYMELSSLRSEDTAVYYCARFYSGSGSPWGQGTLVTVSS

SEQ ID NO: 84 - DNA, 351 bp, Artificial Sequence
  Description of Artificial Sequence Synthetic construct
caggtccagcttgtgcagtctggggctgaggtgaagaagcctggggcctcagtgaaggtttcctgcaaggcttctggatacaccttcactaactatgctatgcattgggtgcgccaggcccccggacaaaggcttgagtggatgggatggatcaacactggcaatggtaacacaaaatattcacagaagttccagggcagagtcaccattaccagggacacatccgcgagcacagcctacatggagctgagcagcctgagatctgaagacacggctgtgtattactgtgcgaggttttactctggttcggggagtccctggggccagggaaccctggtcaccgtctcctca

SEQ ID NO: 85 - protein, 215 aa, Artificial Sequence
  Description of Artificial Sequence Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - DNA, 645 bp, Artificial Sequence
  Description of Artificial Sequence Synthetic construct
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 87 - protein, 108 aa, Artificial Sequence
  Description of Artificial Sequence Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 88 - DNA, 325 bp, Artificial Sequence
  Description of Artificial Sequence Synthetic construct
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggagatcaaac

SEQ ID NO: 89 - protein, 5 aa, Artificial Sequence
  Description of Artificial Sequence Synthetic peptide
NYAMH

SEQ ID NO: 90 - DNA, 15 bp, Artificial Sequence
  Description of Artificial Sequence Synthetic oligonucleotide
aactatgctatgcat

SEQ ID NO: 91 - protein, 17 aa, Artificial Sequence
  Description of Artificial Sequence Synthetic peptide
WINTGNGNTKYSQKFQG

SEQ ID NO: 92 - DNA, 51 bp, Artificial Sequence
  Description of Artificial Sequence Synthetic oligonucleotide
tggatcaacactggcaatggtaacacaaaatattcacagaagttccagggc

SEQ ID NO: 93 - protein, 8 aa, Artificial Sequence
  Description of Artificial Sequence Synthetic peptide
FYSGSGSP

SEQ ID NO: 94 - DNA, 24 bp, Artificial Sequence
  Description of Artificial Sequence Synthetic oligonucleotide
ttttactctggttcggggagtccc

SEQ ID NO: 95 - protein, 12 aa, Artificial Sequence
  Description of Artificial Sequence Synthetic peptide
RASQSVSSSYLA

SEQ ID NO: 96 - DNA, 36 bp, Artificial Sequence
  Description of Artificial Sequence Synthetic oligonucleotide
agggccagtcagagtgttagcagcagctacttagcc

SEQ ID NO: 97 - protein, 7 aa, Artificial Sequence
  Description of Artificial Sequence Synthetic peptide
GASSRAT

SEQ ID NO: 98 - DNA, 21 bp, Artificial Sequence
  Description of Artificial Sequence Synthetic oligonucleotide
ggtgcatccagcagggccact

SEQ ID NO: 99 - protein, 9 aa, Artificial Sequence
  Description of Artificial Sequence Synthetic peptide
QQYGSSPLT

SEQ ID NO: 100 - DNA, 27 bp, Artificial Sequence
  Description of Artificial Sequence Synthetic oligonucleotide
cagcagtatggtagctcaccgctcact

SEQ ID NO: 101 - protein, 30 aa, Artificial Sequence
  Description of Artificial Sequence Synthetic peptide
QVQLVQSGAEVKKPGASVKVSCKASGYTFT

SEQ ID NO: 102 - DNA, 90 bp, Artificial Sequence
  Description of Artificial Sequence Synthetic oligonucleotide
caggtccagcttgtgcagtctggggctgaggtgaagaagcctggggcctcagtgaaggtttcctgcaaggcttctggatacaccttcact

SEQ ID NO: 103 - protein, 23 aa, Artificial Sequence
  Description of Artificial Sequence Synthetic peptide
EIVLTQSPGTLSLSPGERATLSC

SEQ ID NO: 104 - DNA, 69 bp, Artificial Sequence
  Description of Artificial Sequence Synthetic oligonucleotide
gaaattgtgttgacgcagtctccaggcaccctgtctttgtctccaggggaaagagccaccctctcctgc

SEQ ID NO: 105 - protein, 14 aa, Artificial Sequence
  Description of Artificial Sequence Synthetic peptide
WVRQAPGQRLEWMG

SEQ ID NO: 106 - DNA, 42 bp, Artificial Sequence
  Description of Artificial Sequence Synthetic oligonucleotide
tgggtgcgccaggcccccggacaaaggcttgagtggatggga

SEQ ID NO: 107 - protein, 15 aa, Artificial Sequence
  Description of Artificial Sequence Synthetic peptide
WYQQKPGQAPRLLIY

SEQ ID NO: 108 - DNA, 45 bp, Artificial Sequence
  Description of Artificial Sequence Synthetic oligonucleotide
tggtaccagcagaaacctggccaggctcccaggctcctcatctat

SEQ ID NO: 109 - protein, 32 aa, Artificial Sequence; identical to SEQ ID NO: 73
  Description of Artificial Sequence Synthetic peptide

SEQ ID NO: 110 - DNA, 96 bp, Artificial Sequence
  Description of Artificial Sequence Synthetic oligonucleotide
agagtcaccattaccagggacacatccgcgagcacagcctacatggagctgagcagcctgagatctgaagacacggctgtgtattactgtgcgagg

SEQ ID NO: 111 - protein, 32 aa, Artificial Sequence
  Description of Artificial Sequence Synthetic peptide
GIPDRFSGSGSGTDFTLTISRLEPEDFAVYYC

SEQ ID NO: 112 - DNA, 96 bp, Artificial Sequence
  Description of Artificial Sequence Synthetic oligonucleotide
ggcatcccagacaggttcagtggcagtgggtctgggacagacttcactctcaccatcagcagactggagcctgaagattttgcagtgtattactgt

SEQ ID NO: 113 - protein, 11 aa, Artificial Sequence
  Description of Artificial Sequence Synthetic peptide
WGQGTLVTVSS

SEQ ID NO: 114 - DNA, 33 bp, Artificial Sequence
  Description of Artificial Sequence Synthetic oligonucleotide
tggggccagggaaccctggtcaccgtctcctca

SEQ ID NO: 115 - protein, 10 aa, Artificial Sequence
  Description of Artificial Sequence Synthetic peptide
FGGGTKVEIK

SEQ ID NO: 116 - DNA, 30 bp, Artificial Sequence; identical to SEQ ID NO: 44
  Description of Artificial Sequence Synthetic oligonucleotide

SEQ ID NO: 117 - protein, 454 aa, Artificial Sequence
  Description of Artificial Sequence Synthetic construct
EVQLMESGGGLVKPGGSLRLSCAASGFTFSSYSMNWVRQAPGKGLEWVSSITVRSSYIYYADSVKGRFTISRDNAKNSLYLQMNSLRAEDTAVYYCARVLAIAVPGTSYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - DNA, 1362 bp, Artificial Sequence
  Description of Artificial Sequence Synthetic construct
gaggtgcagctgatggagtctgggggaggcctggtcaagcctggggggtccctgagactctcctgtgcagcctctggattcaccttcagtagctatagcatgaactgggtccgccaggctccagggaaggggctggagtgggtctcatccattactgttagaagtagttacatatactacgcagactcagtgaagggccgattcaccatctccagagacaacgccaagaactcactgtatctgcaaatgaacagcctgagagccgaggacacggctgtgtattactgtgcgagagtcctcgctatagcagtgcctggtacctcctactactactacggtatggacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 119 - protein, 127 aa, Artificial Sequence
  Description of Artificial Sequence Synthetic construct
EVQLMESGGGLVKPGGSLRLSCAASGFTFSSYSMNWVRQAPGKGLEWVSSITVRSSYIYYADSVKGRFTISRDNAKNSLYLQMNSLRAEDTAVYYCARVLAIAVPGTSYYYYGMDVWGQGTTVTVSS

SEQ ID NO: 120 - DNA, 381 bp, Artificial Sequence
  Description of Artificial Sequence Synthetic construct
gaggtgcagctgatggagtctgggggaggcctggtcaagcctggggggtccctgagactctcctgtgcagcctctggattcaccttcagtagctatagcatgaactgggtccgccaggctccagggaaggggctggagtgggtctcatccattactgttagaagtagttacatatactacgcagactcagtgaagggccgattcaccatctccagagacaacgccaagaactcactgtatctgcaaatgaacagcctgagagccgaggacacggctgtgtattactgtgcgagagtcctcgctatagcagtgcctggtacctcctactactactacggtatggacgtctggggccaagggaccacggtcaccgtctcctca

SEQ ID NO: 121 - protein, 215 aa, Artificial Sequence
  Description of Artificial Sequence Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - DNA, 645 bp, Artificial Sequence
  Description of Artificial Sequence Synthetic construct
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacggctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 123 - protein, 108 aa, Artificial Sequence
  Description of Artificial Sequence Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRLTFGGGTKVEIK

SEQ ID NO: 124 - DNA, 324 bp, Artificial Sequence
  Description of Artificial Sequence Synthetic construct
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacggctcactttcggcggagggaccaaggtggagatcaaa

SEQ ID NO: 125 - protein, 5 aa, Artificial Sequence
  Description of Artificial Sequence Synthetic peptide
SYSMN

SEQ ID NO: 126 - DNA, 15 bp, Artificial Sequence
  Description of Artificial Sequence Synthetic oligonucleotide
agctatagcatgaac

SEQ ID NO: 127 - protein, 17 aa, Artificial Sequence
  Description of Artificial Sequence Synthetic peptide
SITVRSSYIYYADSVKG

SEQ ID NO: 128 - DNA, 51 bp, Artificial Sequence
  Description of Artificial Sequence Synthetic oligonucleotide
tccattactgttagaagtagttacatatactacgcagactcagtgaagggc

SEQ ID NO: 129 - protein, 18 aa, Artificial Sequence
  Description of Artificial Sequence Synthetic peptide
VLAIAVPGTSYYYYGMDV

SEQ ID NO: 130 - DNA, 54 bp, Artificial Sequence
  Description of Artificial Sequence Synthetic oligonucleotide
gtcctcgctatagcagtgcctggtacctcctactactactacggtatggacgtc

SEQ ID NO: 131 - protein, 12 aa, Artificial Sequence
  Description of Artificial Sequence Synthetic peptide
RASQSVSSSYLA

SEQ ID NO: 132 - DNA, 36 bp, Artificial Sequence; identical to SEQ ID NO: 96
  Description of Artificial Sequence Synthetic oligonucleotide

SEQ ID NO: 133 - protein, 7 aa, Artificial Sequence
  Description of Artificial Sequence Synthetic peptide
GASSRAT

SEQ ID NO: 134 - DNA, 21 bp, Artificial Sequence
  Description of Artificial Sequence Synthetic oligonucleotide
ggtgcatccagcagggccact

SEQ ID NO: 135 - protein, 9 aa, Artificial Sequence
  Description of Artificial Sequence Synthetic peptide
QQYGSSRLT

SEQ ID NO: 136 - DNA, 27 bp, Artificial Sequence
  Description of Artificial Sequence Synthetic oligonucleotide
cagcagtatggtagctcacggctcact

SEQ ID NO: 137 - protein, 30 aa, Artificial Sequence
  Description of Artificial Sequence Synthetic peptide
EVQLMESGGGLVKPGGSLRLSCAASGFTFS

SEQ ID NO: 138 - DNA, 90 bp, Artificial Sequence
  Description of Artificial Sequence Synthetic oligonucleotide
gaggtgcagctgatggagtctgggggaggcctggtcaagcctggggggtccctgagactctcctgtgcagcctctggattcaccttcagt

SEQ ID NO: 139 - protein, 23 aa, Artificial Sequence
  Description of Artificial Sequence Synthetic peptide
EIVLTQSPGTLSLSPGERATLSC

SEQ ID NO: 140 - DNA, 69 bp, Artificial Sequence; identical to SEQ ID NO: 104
  Description of Artificial Sequence Synthetic oligonucleotide

SEQ ID NO: 141 - protein, 14 aa, Artificial Sequence
  Description of Artificial Sequence Synthetic peptide
WVRQAPGKGLEWVS

SEQ ID NO: 142 - DNA, 42 bp, Artificial Sequence
  Description of Artificial Sequence Synthetic oligonucleotide
tgggtccgccaggctccagggaaggggctggagtgggtctca

SEQ ID NO: 143 - protein, 15 aa, Artificial Sequence
  Description of Artificial Sequence Synthetic peptide
WYQQKPGQAPRLLIY

SEQ ID NO: 144 - DNA, 45 bp, Artificial Sequence; identical to SEQ ID NO: 108
  Description of Artificial Sequence Synthetic oligonucleotide

SEQ ID NO: 145 - protein, 32 aa, Artificial Sequence
  Description of Artificial Sequence Synthetic peptide
RFTISRDNAKNSLYLQMNSLRAEDTAVYYCAR

SEQ ID NO: 146 - DNA, 96 bp, Artificial Sequence
  Description of Artificial Sequence Synthetic oligonucleotide
cgattcaccatctccagagacaacgccaagaactcactgtatctgcaaatgaacagcctgagagccgaggacacggctgtgtattactgtgcgaga

SEQ ID NO: 147 - protein, 32 aa, Artificial Sequence; identical to SEQ ID NO: 111
  Description of Artificial Sequence Synthetic peptide

SEQ ID NO: 148 - DNA, 96 bp, Artificial Sequence; identical to SEQ ID NO: 112
  Description of Artificial Sequence Synthetic oligonucleotide

SEQ ID NO: 149 - protein, 11 aa, Artificial Sequence
  Description of Artificial Sequence Synthetic peptide
WGQGTTVTVSS

SEQ ID NO: 150 - DNA, 33 bp, Artificial Sequence; identical to SEQ ID NO: 42
  Description of Artificial Sequence Synthetic oligonucleotide

SEQ ID NO: 151 - protein, 10 aa, Artificial Sequence
  Description of Artificial Sequence Synthetic peptide
FGGGTKVEIK

SEQ ID NO: 152 - DNA, 30 bp, Artificial Sequence; identical to SEQ ID NO: 44
  Description of Artificial Sequence Synthetic oligonucleotide

SEQ ID NO: 153 - DNA, 1086 bp, Macaca fascicularis
atggacaagttttggtggcacgcagcctggggactctgcctcttgcagctgagcctggcgcagatcgatttgaatataacctgccgctttgcgggtgtattccacgtggagaaaaatggtcgctacagcatctctcggacggaggctgctgacctctgcaaggctttcaatagcaccttgcccacaatggcccagatggagaaagctctgagcgtcggatttgagacctgcaggtacgggttcatagaagggcacgtggtgattccccggattcagcccaactccatctgtgcagcaaaccacacaggggtgtacatcctcacgtccaacacctcccagtatgacacatactgcttcaatgcttcagctccacctaaagaagattgtacatcagtcacagacctgcccaatgcctttgatggaccaattaccataactattgttaaccccgatggcactcgctatatcaagaaaggagaatacagaacgaatcctgaagacatctaccccagcaaccctactgatgatgacgtgagcagcggatcctccagtgaaaggagcagcacttcaggaggttacatctttcacaccttttctactgcacaccccatcccagacgaagacggtccctggatcaccgacagcacagacagaatccctgctaccagagaccaagatgcattctaccccagtggggggtcccataccactcatggatctgaatcagctggacactcacatgggagtcaagaaggtggggcaaacacaacctctggtcctgtaaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggatgatagaaagccaagtggactcaatggagaggccagcaagtctcaggaaatggtgcatttggtgaacaaggagccatcagaaactccagaccagtttatgacagctgatgagacaaggaacctgcagaacgtggacatgaagattggggtgtaa

SEQ ID NO: 154 - protein, 525 aa, Homo sapiens [sequence omitted]

SEQ ID NO: 155 - DNA, 30 bp, Artificial Sequence
  Description of Artificial Sequence Synthetic primer
atggacaagttttggtggcacgcagcctgg

SEQ ID NO: 156 - DNA, 25 bp, Artificial Sequence
  Description of Artificial Sequence Synthetic primer
ttacaccccaatcttcatgtccaca

SEQ ID NO: 157 - DNA, 33 bp, Artificial Sequence
  Description of Artificial Sequence Synthetic primer
gactcgaggccaccatggacaagttttggtggc

SEQ ID NO: 158 - DNA, 36 bp, Artificial Sequence
  Description of Artificial Sequence Synthetic primer
gatctagatcactattacaccccaatcttcatgtcc

SEQ ID NO: 159 - DNA, 18 bp, Artificial Sequence
  Description of Artificial Sequence Synthetic primer
atggacaagttttggtgg

SEQ ID NO: 160 - DNA, 24 bp, Artificial Sequence
  Description of Artificial Sequence Synthetic primer
gttacaccccaatcttcatgtcca

SEQ ID NO: 161 - DNA, 43 bp, Artificial Sequence
  Description of Artificial Sequence Synthetic primer
agtgagactagtcagatcgatttgaatataacctgccgctttg

SEQ ID NO: 162 - DNA, 38 bp, Artificial Sequence
  Description of Artificial Sequence Synthetic primer
atcactgagatcttctggaatttggggtgtccttatag

SEQ ID NO: 163 - DNA, 31 bp, Artificial Sequence
  Description of Artificial Sequence Synthetic primer
atcggcgatccagatcgatttgaatataacc

SEQ ID NO: 164 - DNA, 35 bp, Artificial Sequence
  Description of Artificial Sequence Synthetic primer
ctgtgcctcgagccattctggaatttggggtgtcc

SEQ ID NO: 165 - DNA, 25 bp, Artificial Sequence
  Description of Artificial Sequence Synthetic primer
attyrgtgatcagsactgaacasag

SEQ ID NO: 166 - DNA, 20 bp, Artificial Sequence
  Description of Artificial Sequence Synthetic primer
tacgtgccaagcatcctcgc

SEQ ID NO: 167 - DNA, 24 bp, Artificial Sequence
  Description of Artificial Sequence Synthetic primer
atcaatgcctgkgtcagagcyytg

SEQ ID NO: 168 - DNA, 23 bp, Artificial Sequence
  Description of Artificial Sequence Synthetic primer
aggctggaactgaggagcaggtg

SEQ ID NO: 169 - DNA, 24 bp, Artificial Sequence
  Description of Artificial Sequence Synthetic primer
ccctgagagcatcaymyarmaacc

SEQ ID NO: 170 - DNA, 20 bp, Artificial Sequence
  Description of Artificial Sequence Synthetic primer
tacgtgccaagcatcctcgc

SEQ ID NO: 171 - DNA, 26 bp, Artificial Sequence
  Description of Artificial Sequence Synthetic primer
gsartcagwcycwvycaggacacagc

SEQ ID NO: 172 - DNA, 23 bp, Artificial Sequence
  Description of Artificial Sequence Synthetic primer
aggctggaactgaggagcaggtg

SEQ ID NO: 173 - DNA, 24 bp, Artificial Sequence
  Description of Artificial Sequence Synthetic primer
ccctgagagcatcaymyarmaacc

SEQ ID NO: 174 - DNA, 20 bp, Artificial Sequence
  Description of Artificial Sequence Synthetic primer
tacgtgccaagcatcctcgc

SEQ ID NO: 175 - DNA, 24 bp, Artificial Sequence
  Description of Artificial Sequence Synthetic primer
atcaatgcctgkgtcagagcyytg

SEQ ID NO: 176 - DNA, 23 bp, Artificial Sequence
  Description of Artificial Sequence Synthetic primer
aggctggaactgaggagcaggtg

SEQ ID NO: 177 - DNA, 33 bp, Artificial Sequence
  Description of Artificial Sequence Synthetic primer
aaggcttctggatacaccttcactagctatgct

SEQ ID NO: 178 - DNA, 33 bp, Artificial Sequence
  Description of Artificial Sequence Synthetic primer
agcatagctagtgaaggtgtatccagaagcctt

SEQ ID NO: 179 - DNA, 33 bp, Artificial Sequence
  Description of Artificial Sequence Synthetic primer
ttagcctggtatcagcagaaaccagggaaagcc

SEQ ID NO: 180 - DNA, 33 bp, Artificial Sequence
  Description of Artificial Sequence Synthetic primer
ggctttccctggtttctgctgataccaggctaa

SEQ ID NO: 181 - DNA, 23 bp, Artificial Sequence
  Description of Artificial Sequence Synthetic primer
caggtgcagctggtggagtctgg

SEQ ID NO: 182 - DNA, 22 bp, Artificial Sequence
  Description of Artificial Sequence Synthetic primer
tggaggctgaggagacggtgac

SEQ ID NO: 183 - DNA, 25 bp, Artificial Sequence
  Description of Artificial Sequence Synthetic primer
gaaattgtgttgacacagtctccag

SEQ ID NO: 184 - DNA, 53 bp, Artificial Sequence
  Description of Artificial Sequence Synthetic primer
tatattccttaattaagttattctactcacgtttgatctccaccttggtccct

SEQ ID NO: 185 - DNA, 22 bp, Artificial Sequence
  Description of Artificial Sequence Synthetic primer
caggtccagcttgtgcagtctg

SEQ ID NO: 186 - DNA, 22 bp, Artificial Sequence
  Description of Artificial Sequence Synthetic primer
tggaggctgaggagacggtgac

SEQ ID NO: 187 - DNA, 23 bp, Artificial Sequence
  Description of Artificial Sequence Synthetic primer
gacatccagatgacccagtctcc

SEQ ID NO: 188 - DNA, 53 bp, Artificial Sequence
  Description of Artificial Sequence Synthetic primer
tatattccttaattaagttattctactcacgtttgatttccaccttggtccct

SEQ ID NO: 189 - DNA, 22 bp, Artificial Sequence
  Description of Artificial Sequence Synthetic primer
caggtccagcttgtgcagtctg

SEQ ID NO: 190 - DNA, 22 bp, Artificial Sequence
  Description of Artificial Sequence Synthetic primer
tggaggctgaggagacggtgac

SEQ ID NO: 191 - DNA, 25 bp, Artificial Sequence
  Description of Artificial Sequence Synthetic primer
gaaattgtgttgacgcagtctccag

SEQ ID NO: 192 - DNA, 53 bp, Artificial Sequence; identical to SEQ ID NO: 184
  Description of Artificial Sequence Synthetic primer

SEQ ID NO: 193 - protein, 118 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARYYYYYGMDVWGQGTTVTVSS

SEQ ID NO: 194 - protein, 107 aa, Homo sapiens
EIVLTQSPATLSLSPGERATLSCRASQSVSSYLAWYQQKPGQAPRLLIYDASNRASGIPARFSGSGSGTDFTLTISSLEPEDFAVYYCQQRSNWPLTFGGGTKVEIK

SEQ ID NO: 195 - protein, 118 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARYYYYYGMDVWGQGTTVTVSS

SEQ ID NO: 196 - protein, 107 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPWTFGQGTKVEIK

SEQ ID NO: 197 - protein, 117 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARYYYGSGSPWGQGTLVTVSS

SEQ ID NO: 198 - protein, 108 aa, Homo sapiens; identical to SEQ ID NO: 87

SEQ ID NO: 199 - protein, 126 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAREGIAVAGTYYYYYGMDVWGQGTTVTVSS

SEQ ID NO: 200 - protein, 108 aa, Homo sapiens; identical to SEQ ID NO: 87